SEQUENCE LISTING (2 sequences)

SEQ ID NO: 1 - protein, 18 aa, synthetic construct
KKKKKKKKKKSIINFEKL

SEQ ID NO: 2 - protein, 8 aa, synthetic construct
SIINFEKL

(19) United States
(12) Patent Application Publication
(10) Pub. No.: US 2026/0053906 A1
(43) Pub. Date: Feb. 26, 2026

(54) DNA ORIGAMI VACCINES

(71) Applicant: OHIO STATE INNOVATION FOUNDATION, Columbus, OH (US)

(72) Inventors: Niksa ROKI, Columbus, OH (US); Carlos CASTRO, Columbus, OH (US); Christopher LUCAS, Columbus, OH (US); Meixiao LONG, Columbus, OH (US); John BYRD, Columbus, OH (US)

(21) Appl. No.: 19/105,577

(22) PCT Filed: Aug. 24, 2023
(86) PCT No.: PCT/US2023/072801

§ 371 Date: Feb. 21, 2025

Related U.S. Application Data
(60) Provisional application No. 63/373,489, filed on Aug. 25, 2022.

(51) Int. Cl. A61K 39/12; A61K 9/51; A61K 39/00; A61K 39/39; A61K 47/26; A61K 47/69; B82Y 5/00

(52) U.S. Cl. CPC A61K 39/12; A61K 9/513; A61K 39/0011; A61K 39/39; A61K 47/26; A61K 47/6921; B82Y 5/00; A61K 2039/55561; A61K 2039/6025; A61K 2039/62

(57) ABSTRACT

Disclosed herein is a vaccine device that involves a DNA origami nanostructure formed from a plurality of scaffold strands and a plurality of staple strands assembled into a rod shape, wherein a peptide antigen is attached to the DNA of the nanostructure by electrostatic interaction. Also disclosed herein is a method for vaccinating a subject that involves administering to the subject a therapeutically effective amount of a vaccine device disclosed herein.

### CROSS-REFERENCE TO RELATED APPLICATIONS

This application claims benefit of U.S. Provisional Application No. 63/373,489, filed Aug. 25, 2022, which is hereby incorporated herein by reference in its entirety.

### STATEMENT REGARDING FEDERALLY SPONSORED RESEARCH OR DEVELOPMENT

This invention was made with Government Support under Grant Nos. CA197734 and HL141941 awarded by the National Institutes of Health. The Government has certain rights in the invention.

### SEQUENCE LISTING

This application contains a sequence listing filed in ST.26 format entitled “321501_2650_Sequence_Listing” created on Aug. 23, 2023, having 2,574 bytes. The content of the sequence listing is incorporated herein in its entirety.

### BACKGROUND

Cancer vaccines contain tumor antigens designed to stimulate APCs and cytotoxic T cells that can eradicate tumors, while providing immunological memory to prevent relapse (Houghton, A N & Guevara-Patiño, J A. J Clin Invest 2004 114:468-471). Unlike prophylactic vaccines that are generally administered against specific and highly immunogenic non-self antigens, and to individuals with a healthy functioning immune system, tumor vaccines require overcoming several challenges associated with the complex and less understood interplay between the immune system and tumor microenvironment (Houghton, A N & Guevara-Patiño, J A. J Clin Invest 2004 114:468-471; Fleming, V, et al. Front. Immunol. 2018 9). These challenges include safety concerns, poor immunogenicity and specificity of tumor antigens, immune suppression by the tumor, and tumor antigen heterogeneity, which ultimately lead to suboptimal immune responses against cancer (Lou, J, et al. Advanced Therapeutics 2019 2:1800128). To overcome these challenges, novel delivery technologies that can integrate and precisely synchronize multiple immune regulating functions and a broad spectrum of antigens for robust tumor-specific immunogenicity are required.

### SUMMARY

Disclosed herein is a vaccine device that involves a DNA origami nanostructure formed from a plurality of scaffold strands and a plurality of staple strands assembled into a rod shape, wherein a peptide antigen is attached to the DNA of the nanostructure by electrostatic interaction.

In some embodiments, the peptide antigen has at least 5, 6, 7, 8, 9, or 10 contiguous positively charged amino acids to facilitate the electrostatic interaction. For example, the at least 5, 6, 7, 8, 9, or 10 continuous positively charged amino acids can be at the N-terminus of the peptide antigen, the C-terminus of the peptide antigen, or a combination thereof.

In some embodiments, the positively charged amino acids are lysine and/or arginine amino acids. The vaccine device of claim 2 or 3, wherein the peptide antigen comprises at least 10 contiguous lysine amino acids.

In some embodiments, at least 500, 1,000, 2,000, 3,000, 4,000, or 5,000 peptide antigens are attached to the DNA nanostructure. For example, in some embodiments, there are at least 30, 35, 40, 45, 50, 60, 70, 80, 90, or 100 peptides per nm2. In some embodiments, each vaccine device contains a peptide concentration of at least 50, 60, 70, 80, 90, 100 nM.

In some embodiments, each of the plurality of scaffold strands are 5,000 to 10,000 nucleotides in length, including about 5,000, 6,000, 7,000, 8,000, 9,000, or 10,000 nucleotides in length. In some embodiments, each of the plurality of scaffold strands are derived from a virus, such as bacteriophage M13.

In some embodiments, the peptide antigen involves a viral antigen. In some embodiments, the peptide antigen involves a tumor associated antigen.

In some embodiments, the DNA origami nanostructure involves one or more first single stranded DNA oligonucleotide attachment arms configured to bind to a first complementary DNA oligonucleotide strands.

In some embodiments, the DNA origami nanostructure further involves a branched oligonucleotide dendrimer having a plurality of second single stranded DNA oligonucleotide attachment arms and at least one first complementary DNA oligonucleotide strand, wherein the second single stranded DNA oligonucleotide attachment arms are configured to bind to second complementary DNA oligonucleotide strands.

In some embodiments, the DNA origami nanostructure further involves a plurality of adjuvant molecules, such as nucleic acid adjuvant molecules, conjugated to the first complementary DNA oligonucleotide strands or second complementary DNA oligonucleotide strands.

For example, the plurality of adjuvant molecules can be a toll like receptor (TLR) agonist. Examples of TLR agonists are described in Yang, J-X. Y., et al. Pharmaceutics. 2022 14(2): 423, which is incorporated by reference in its entirety for the teaching of these agonists and their uses as adjuvants. For example, in some embodiments, the TLR agonist is lipopolysaccharide (LPS), polyinosinic: polycytidylic acid (poly I: C), lipoteichoic acid (LTA), flagellin, imidazoquinoline, I-isobutyl-I H-imidazo[4,5-c]quinoline-4-amine, or a CpG oligonucleotide. In some embodiments, the TLR agonist is GARDIQUIMOD™ (CAS number 1020412-43-4) or 1-isobutyl-1H-imidazo[4,5-c]quinoline-4-amine. In some embodiments, the TLR agonist is IFNγ, IL-6, or phorbol 12-myristate 13-acetate (PMA).

In some embodiments, the rod shape of the DNA origami nanostructure has a cavity, wherein the one or more first single stranded DNA oligonucleotide attachment arms are positioned inside the cavity. For example, the cavity can be approximately 3, 4, 5, 6, or 7 nanometers in diameter.

In some embodiments, the vaccine device further involves one or more targeting ligands conjugated to the first complementary DNA oligonucleotide strands or second complementary DNA oligonucleotide strands. For example, the one or more targeting ligands can be DNA aptamers, peptide aptamers, or antibodies. In some embodiments, the targeting ligand targets dendritic cells (DCs). Examples of suitable targeting ligands are described in Apostolopooulos, V, et al. J Drug Deliv. 2013 2013:869718, which is incorporated by reference for the teaching of these targeting antigens and their uses for vaccines. Numerous receptors have been identified to be expressed on DCs, including mannose receptor (MR), DC-SIGN, scavenger receptor (SR), DEC-205, and toll-like receptors. For example, the targeting ligand can be an aptamer that binds DEC-205.

In some embodiments, each branched oligonucleotide dendrimer has at least 10, 11, 12, 13, 14, 15, 16, 17, 18, 19, 20, 25, 30, 35, 40, 45, 50, 60, 70, 80, 90, or 100 second single stranded DNA oligonucleotide attachment arms. The number of branches that can be achieved depends on the volume wherein the theoretical maximum approaches the density of DNA origami nanostructures. This depends on the length of the branches.

In some embodiments, the DNA origami nanostructure is encapsulated in an alginate capsule for therapeutic delivery.

Also disclosed herein is a method for vaccinating a subject that involves administering to the subject a therapeutically effective amount of a vaccine device disclosed herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic pertaining to an embodiment of a workflow towards optimizing a DNA Origami (DO)-based vaccine. Various DO-based vaccine formulations were designed and tested for their geometry, stability, using antigen presenting cells such as dendritic cells, and T cells and their cytotoxic function that is useful against treatment of viruses and cancers. An embodiment of the disclosed DO contains free oligonucleotide attachment arms. These arms are useful for attaching CpG adjuvants, other nucleotide-based drugs, or any molecule that can be directly conjugated to these arms or conjugated to oligonucleotide arms complementary to the attachment arms of DO. Next, peptide antigen can be assembled containing 10 lysine amino acids with amine groups. Once antigen is included onto DO, this becomes a vaccine formulation referred to herein as DOVAC. A formulation without an antigen is not a vaccine, rather simply an adjuvant or a carrier of particular therapy.

FIG. 2 is a schematic pertaining to an embodiment of DO vaccine formulations. Aim 1 describes basic DOVAC formulation with peptide antigen loading method. Aim 2 describes modulation of geometry (size and shape), modulation of ligand-targeting parameters, including a utilization of a branched design to increase the number and flexibility of presentation of targeting ligands. Further is shown incorporation of adjuvants into DO cavity to improve masking of adjuvants and reduce their release in circulation, which may improve vaccine safety. Next, shown is a concept design of branch assembly in the internal cavity to increase the attachment density inside DO. Lastly, alginate or other polymers can be used to encapsulate DOVAC for extended release properties.

FIG. 3 contains schematics pertaining to an embodiment of DO vaccine protein-based formulation. Presented are peptide subunit DOVAC and protein subunit DOVAC utilizing branched design to allow for high adjuvant, protein antigen, and ligand targeting attachment capacity. The branched DO design can allow for targeting ligands to multiple target epitopes and to multiple cell types (e.g., dendritic cells, macrophages, B cells, high endothelial venules, other endothelial cells, etc.) at a single DO NP without compromising on the sufficient number of attached ligands or molecules, particularly for the ligands of relatively low affinity such as aptamers. Combination targeting to different target epitopes on the same cell type, as well as targeting multiple cell types is a known strategy to reduce off-site targeting, and branched DO design may improve this strategy. Lastly, high coating of DO, particularly with proteins such as antibodies has been shown to protect DNA nanoparticle half-life in serum and in the body.

FIG. 4 shows an embodiment of DO that acts as an adjuvant and induces dendritic cell activation. Dendritic cells (cell line MUTU1940DCs) were incubated with PBS buffer containing MgCl₂ (untreated control) or treated with free CpG at 266 nM (an amount corresponding to loading 64 arms on DO administered at 4.15 nM), scaffold at 4.15 nM, or DO at 4.15 nM for 24 h. The CpG and CpG/DO controls showed that the majority of activation was due to the DO adjuvancy by itself, and not due to the attached CpG.

FIG. 5A shows Novavax NVX-CoV2373 vaccine design. FIG. 5B shows Novavax NVX-CoV2373 NPs are not uniform, and adjuvant is not complexed with the antigen, potentially leading to suboptimal immunization and increasing the risk of tolerization regarding cancer vaccines.

FIGS. 6A to 6F show assembly of an embodiment of a high payload capacity DO branched oligonucleotide arms for attachment of various numbers of molecules of interest. FIG. 6A shows agarose electrophoresis gels indicating successful manufacturing of increasing number of attachment arms and subsequent CpG attachment based on the band shifts at each step of synthesis. Tight bands indicate precise and uniform product. DO30 and DO65 contain 30 and 65 attachment arms and utilize common technique of designing attachment arms or overhangs in DNA origami field. DO90, DO150, and DO320 are decorated with 90, 150, or 320 attachment arms respectively and are products of subsequent addition of DNA oligonucleotides designed using specific design rules to result in complete and efficient assembly. These rules include limiting self-binding sequence regions, limiting non target oligonucleotide binding, etc. FIG. 6B are transmission electron microscopy (TEM, left) and atomic force microscopy (AFM, right) of DO or DO65 showing uniform geometry, but attachment arms are hard to visualize. FIGS. 6C and 6D show DO65 decorated with branched attachment arms, where branched arms are clearly visible on TEM (FIG. 6C) and AFM (FIG. 6D). The flexibility of branches on DO surface as indicated some of the branches being bent during drying process. FIG. 6E shows quantification of attachment of model molecule (fluorophore labeled CpG) to regular or branched design attachment arms shows high attachment efficiency over 94% and increasing payload capacity for increasing number of arm attachment sites. Fluorescence intensity and DNA optical density measurements were used to determine concentration standard curve and fluorescence intensity for each sample after purification to determine the number of molecules attached per single NP. F) Example of model protein attachment (antibodies) to demonstrate attachment to DO30 design and feasibility of protein subunit vaccine production with precise adjuvant (i.e., DO) geometry and efficient loading of additional CpG adjuvant molecules.

FIGS. 7A and 7B show high efficiency and payload capacity of peptide antigen loading onto DNA origami nanoparticles. FIG. 7A shows efficient adjuvant attachment and the loading of up to 2900 antigen molecules per NP was confirmed by fluorescent gel characterization by tracing colocalization of FAM-OVA and Atto-647-oligonucleotide on DO NPs. The higher MW DO NPs modified with branched oligonucleotides showed higher peptide payload capacity. FIG. 7B shows TEM used to confirm that the morphology is still intact after antigen coating.

FIGS. 8A and 8B show DO-VAC protects antigen and adjuvant from degradation. FIG. 8A shows DOVAC was stable in 95% serum for up to 24 h. FIG. 8B shows that by using a more sensitive gel imager, DO and adjuvant were stable up to 24 h to 48 h, while antigen was stable up to 5 h with minor degradation. DO protected antigen from degradation as compared to free antigen. Even at 48 h, a majority of antigen is still protected and adsorbed to DO. (V stands for DO-VAC.)

FIGS. 9A and 9B show DOVAC increases antigen presentation in dendritic cells in vitro. FIG. 9A shows flow cytometry demonstrated similar peptide antigen presentation via MHCl of DO-VAC vs. physical mixtures (OVA+CpG or DO+OVA) and other controls at 3 h post incubation (not shown), with advantage of DO-VAC at the 12 h-point. This is likely due to the DO-mediated resistance to degradation, protection of surface antigen, and high payload delivery. FIG. 9B shows the number of attachment arms or adjuvant attachment had no effect on antigen presentation. (p<0.05, Student's t-test). Please note that flow cytometry antibody was used that specifically detects OVA peptide complexed with MHCl, but not free OVA peptide bound elsewhere on cell or DO surface.

FIGS. 10A to 10C show DOVAC increases T cell expansion and activation in vivo. FIG. 10A shows in vivo evaluation of OVA-specific CD8 T cells after their transfer into wild type mice demonstrated upregulation of CD69 (early activation marker) for DOVAC group as compared to the untreated control or standard OVA protein/Complete Freund's Adjuvant (OVA/CFA) vaccine, and also increased CD25 (T cell activation marker) expression on CD8 T cells one day post treatment. FIG. 10B shows three days post treatment, DOVAC induced distinct expansion profile of adoptively transferred OVA-specific T cells in vivo and a greater number of cell divisions for a large fraction of cells as compared to administration of mixture of free CpG adjuvant and OVA antigen peptide (CpG in these experiments was a potent version of CpG, different than shown for in vitro data). FIG. 10C shows in the same experiment as in FIG. 10B, DOVAC showed a drastic increase in CD25 activation marker as compared to much lower increase for mixture of free CpG and OVA peptide as compared to untreated (buffer) control.

FIGS. 11A and 11B show aptamer targeted DOVAC to dendritic cells using DEC205 ligand for more effective vaccines. FIG. 11A shows increased DOVAC delivery to dendritic cells when utilizing dendritic cell targeting to DEC205 as compared to non-targeted DOVAC. FIG. 11B shows increased DOVAC adjuvancy when utilizing dendritic cell targeting via DEC205 as compared to non-targeted DOVAC.

FIG. 12 shows polymer encapsulation strategy of DOVAC for controlled vaccine release. Fluorescent images show colocalization of FAM-labeled OVAK10 antigen peptide, Cy3-labeled DO, and Atto647-labeled CpG adjuvant. Shown is also a corresponding bright field image. A graph showing dendritic cells continue to be stimulated by alginate encapsulated DOVAC even at 7 days post administration in vitro, but not free DOVAC.

FIG. 13 shows an experimental design schematic for immunogenicity evaluation of Triangle and Horse DNA origami nanostructures in vivo. Female ICR mice were subjected to a repeat-dosing regimen: 5 total injections administered (i.p.) at 48-hr intervals over a 10-day time course with the following treatment groups: (PBS+2.5 mM MgCl₂, (n=4) (blue); CpG-ODN (10 μg, n=5) (red); M13mp18 (6.0 mg/kg, n=7); (yellow) AF750-Tri DNA origami (DO) (12.0 mg/kg, n=7) (brown); and AF750-Horse DNA origami (12.0 mg/kg, n=7) (green). The number of animals chosen for each treatment group was suggested by previous statistical power analysis results. The lower animal numbers represent control conditions previously studied, where the higher numbers represent experimental treatment groups. Peripheral blood was collected for complete blood counts (CBCs) and plasma cytokine levels on days 0, 1, 3, 5, and 10. On day 10 (endpoint) peripheral blood was also collected for plasma antibody levels via ELISA. Peripheral blood and splenic mononuclear were analyzed via flow cytometry.

FIGS. 14A to 14E show immunogenicity of DO nanostructures in vitro and in vivo. To identify whether early immune activation occurred, splenic mononuclear cells were cultured with either PBS+ or AF750-labeled Tri and Horse DO (100 nM) for 1, 5, and 24 hrs followed by flow cytometry to assess the level of BUV395-anti-CD69 fluorescence among the CD11b, CD11c, NK1.1, CD19, and CD3 immune cell populations. FIG. 14A shows the percentage of cells positive for DO and CD69 expressed as mean BUV395-CD69% Gated±SEM and represent three independent experiments. FIG. 14B shows in vivo CBC analysis of white blood cells (WBCs) right, monocytes (middle), and lymphocytes (right) where dashed lines represent upper and lower normal range boundaries. FIG. 14C shows peripheral blood collected for complete blood counts (CBCs) on days 0, 1, 3, 5, and 10. CBC analysis of neutrophils (top), eosinophils (middle), and basophils (bottom) where dashed lines represent upper and lower normal range boundaries and arrows indicate injection (i.p.) days. FIG. 14D shows plasma cytokine evaluation of Triangle and Horse DNA origami nanostructures in vivo. Female ICR mice underwent a repeat dosing regimen with treatment groups described in FIG. 13. Plasma isolated from peripheral blood collected on days 0, 1, 3, 5, and 10 was analyzed for a panel of cytokine/chemokine levels including inflammatory mediators (IFN-γ, IL-1β, IL-6, IL-10, IL-12 (p70), TNF-α) chemoattractants (MCP-1, MIP-2, MIG) and T-cell cell mediators (IL-2, IL-5, IL-17) via the Milliplex Magnetic Cytokine array. The data are expressed as mean fold change (pg/ml) for each cytokine/chemokine. The following animal number was analyzed per treatment group: (PBS+, n=2-3; CpG-ODN, n=4; M13mp18, n=5-6; Tri DO, n=5-6; and Horse DO, n=6-7). FIG. 14E shows IgM and IgG plasma levels in response to Triangle and Horse DNA origami nanostructures in vivo. Female ICR mice were subjected to a repeat-dosing regimen (as described in FIG. 13) where peripheral blood was collected on days 0 and 10 to evaluate plasma levels of IgM and IgG. IgM and IgG ELISAs of were performed on the following samples (Tri, n=3; Horse, n=4) (other treatment groups not included due to availability) collected on days 0 and 10. The data in are expressed as mean IgM (μg/ml) or mean IgG (μg/ml)±SEM.

FIG. 15 shows early immune activation in peripheral blood mononuclear cells in vitro. To establish a positive control for CD69 elevation, whole peripheral blood was cultured in the presence of PBS+ or CpG-ODN (˜1 μM) for 6 hrs followed by flow cytometry to assess CD69 surface level expression on the CD11b, CD11c, NK1.1, CD19, CD3, CD4, and CD8 positive cell populations. Fluorescent minus one (FMO) flow cytometry control samples were included that contained all fluorophore labeled antibodies for CD45, CD11b, CD11c, NK1.1, CD19, CD3, CD4, and CD8 antigens except for BUV-395-CD69. The data are shown as histograms of % Gated vs. BUV395-CD69 fluorescence (log scale) and include the following color scheme: PBS+ (FMO), CpG-ODN (FMO), PBS+, and CpG-ODN. The comparable signal among PBS+ and CpG-ODN (FMO) samples suggests that the BUV-395-CD69 antibodies are binding to the cell surface in a specific manner, while the present right shift of CpG-ODN treated cells suggests CD69 elevation.

FIG. 16 shows DO immune cell localization in vitro. To identify the immune cell subtype(s) responsible for internalizing DO nanostructures in vitro and whether early immune activation occurred, splenic mononuclear cells were cultured with either PBS+ or AF750-labeled Tri and Horse DO (100 nM) for 1, 5, and 24 hrs followed by flow cytometry to assess the level of AF750-DO among the CD11b, CD11c, NK1.1, CD19, and CD3 immune cell populations. The percentage of cells positive for DO and CD69 are expressed as mean DO-AF750% Gated±SEM. Stats, two-way ANOVA followed by Tukey's Multiple Comparisons test where statistical differences are shown between groups. *, p≤0.05; **, p≤0.01; ***, p≤0.001; ****, p<0.0001.

FIGS. 17A to 17D show the biodistribution profile of Triangle (Tri) and Horse DNA origami nanostructures in vivo. Animals (at 2 hrs (i.v. and i.p) and 4 hrs (i.p.)) post injection were euthanized, necropsied, and major organ systems were visualized via IVIS as described and labeled as follows: i. brain; ii. lung(s); iii. liver; iv. spleen; v. hind legs (bone marrow); vi. heart; vii. stomach; viii. kidneys; ix. reproductive (ovaries, fallopian tubes, uterus); x. urine; xi. peripheral blood. FIGS. 17A to 17D show a representative example of organs, urine and blood from an AF750-staple treated mouse (i.v.) (FIG. 17A); from i.v. treated mice at 2 hrs (FIG. 17B); from i.p. treated mice at 2 hrs (FIG. 17C); and from i.p. treated mice at 4 hrs (FIG. 17D). The data are expressed as the mean Radiant Efficiency [p/s/cm²/sr]/[μW/cm²]±SEM and represent 6 (i.v. treated (PBS+, DO)), 3-4 (i.p. treated (PBS+, DO)) and 3 (i.v. and i.p. treated (PBS+, AF750-staples)) independent experiments. At 2 hrs post i.v. injection, the Alexa750 oligonucleotides and Alexa750-labeled Tri- and Horse-DO were localized mainly to the urine, kidneys, and the liver, where an increased Tri-DO signal was evident relative to Horse-DO in each tissue and detectable Horse- and Tri-DO levels were found in the reproductive organs, additionally, an increased Tri-DO liver signal relative to Horse-DO may suggest a slower clearance rate.

FIGS. 18A to 18D show a structural evaluation of Triangle (Tri) and Horse DNA origami nanostructures. FIG. 18A shows a solid model schematic of Triangle (Tri) and Horse DNA origami (DO) nanostructures (top) and color code Tri (brown) and Horse (green) used in the current study. FIG. 18B shows agarose gel electrophoresis (AGE) post construction of Tri and Horse DO labeled with AlexaFluor (AF)-750 where the lanes are as follows (left to right): 1 kb Molecular Weight Ladder, M13mp18 scaffold DNA, Tri-AF750, and Horse-AF750 (top, typhoon gel fluorescence, 640 nm excitation; bottom, ethidium bromide, UV excitation). The gel was cropped to show relevant Tri and Horse DO lanes next to each other. FIG. 18C shows gel-purified Tri and Horse DO subjected to transmission electron microscopy (TEM) and atomic force microscopy (AFM) showing well-formed Tri and Horse DO. FIG. 18D shows Tri and Horse DO were added (1:1) with non-heat inactivated FBS and incubated over a 24-hr time course, followed by PEG-purification and AGE to assess structural stability in vitro; lanes are as follows (left to right): 1kb Molecular Weight Ladder, M13mp18 scaffold DNA, Tri and Horse DO alone, and Tri and Horse DO incubated with FBS (50% solution) for 0, 1, 2, 6, and 24 hrs. Representative images in FIGS. 18B-18D are shown from at least three independent experiments. Scale bars, 50 nm.

FIGS. 19A and 19B show an evaluation of endotoxin levels in M13mp18 scaffold DNA samples. M13mp18 scaffold DNA was propagated and isolated both with and without the EndoFree Maxi Prep purification kit (Qiagen) followed by endotoxin level evaluation by the ToxinSensor™ Chromogenic LAL Endotoxin Assay (GenScript). 10 samples (˜880 nM) isolated on 10 different days were pooled together and diluted 100-fold (1:100) followed by a 5-fold serial dilution (1:500; 1:2500; and 1:12500) with 1× TE buffer and subjected to the LAL Endotoxin Assay. FIG. 19A shows a standard curve of 0.1, 0.05, 0.025, and 0.00125 endotoxin units (EU)/ml where linear regression analysis was conducted to generate a linear equation (left) and samples (in triplicate) mean OD 545 nm±SEM (right). FIG. 19B shows tables showing measured (mean OD 545 nm±SEM) and solved unknown endotoxin levels from samples where the Endo Toxin removal process was not used (top) and used (bottom). Top, increasing solved unknown endotoxin levels suggest a saturated plate where the level of endotoxin is ≥ 3098 EU/ml. Bottom, negative values for solved endotoxin levels suggest below the level of assay detection.

FIGS. 20A to 20C show a toxicology evaluation of Triangle (Tri) and Horse DNA origami nanostructures in vivo. Female ICR mice were subjected to a repeat-dosing regimen as described in FIG. 13: 5 total injections administered (i.p.) at 48-hr intervals over a 10-day time course with the following treatment groups: (PBS+2.5 mM MgCl₂, (n=4); CpG-ODN (10 μg, n=5); M13mp18 (6.0 mg/kg, n=7); AF750-Tri DNA origami (DO) (12.0 mg/kg, n=7); and AF750-Horse DNA origami (12.0 mg/kg, n=7). FIG. 20A show weight monitored daily and expressed as weight (g)±SEM. FIG. 20B shows histopathology of liver and kidney sections at endpoint (day 10) where arrows indicate mild hepatic necrosis (heart, uterus, and urinary bladder sections) where a representative image is shown from n=4 mice/treatment group, (20× magnification). FIG. 20C shows complete biochemistry panel monitoring liver/kidney function: Albumin (ABL), Alanine Aminotransferase (ALT), Aspartate Aminotransferase (AST), Total Protein (TPROT), Glucose (GLU), Total Bilirubin (TBIL), Blood Urea Nitrogen (BUN), and Creatinine (CREAT). Dashed lines represent upper bound of normal range. The data are expressed as mean (parameter concentration)±SEM from n=4 mice/treatment group. Stats, two-way ANOVA followed by Tukey's Multiple Comparisons test (FIG. 20A) and one-way ANOVA followed by Dunnett's Multiple Comparisons test (FIG. 20C) where statistical differences are shown relative to the PBS+ treated mice. N.S., not significant; *, p≤0.05; **, p≤0.01.

FIG. 21 shows a histopathology evaluation of Triangle (Tri) and Horse DNA origami nanostructures in vivo. Female ICR mice were subjected to a repeat-dosing regimen as described in FIG. 13: 5 total injections administered (i.p.) at 48-hr intervals over a 10-day time course with the following treatment groups: (PBS+2.5 mM MgCl₂, (n=4); CpG-ODN (10 μg, n=5); M13mp18 (6.0 mg/kg, n=7); AF750-Tri DNA origami (DO) (12.0 mg/kg, n=7); and AF750-Horse DNA origami (12.0 mg/kg, n=7). Histopathology of heart, uterus, and urinary bladder sections at endpoint (day 10). A representative image is shown from n=4 mice/treatment group. (20× magnification)

FIG. 22 shows a complete biochemistry toxicology profile. Female ICR mice subjected to a repeat-dosing regimen as described in FIG. 13. Complete biochemistry panel monitoring liver/kidney function: alkaline phosphatase (ALP); gamma-glutamyl transferase (GGT); creatine kinase (CK); globulin (GLOB); amylase (AMYL); lipase (LIP); BUN-to-creatine ratio (BUNCRE); cholesterol (CHOL); triglycerides (TRIG); calcium (CA); potassium (K); sodium (NA); phosphorus (PHOS). The data are expressed as mean (parameter concentration)±SEM from n=1-4 mice/treatment group. Stats, one-way way ANOVA followed by Dunnett's Multiple Comparisons test where statistical differences are shown relative to the PBS+ treated mice. *, p<0.05.

FIG. 23 shows the DO-VAC proof-of-concept approach. (1) immunogenicity of the DO-VAC platform will be assessed in vitro and in healthy mice in vivo. (2) DO-VAC anti-tumor efficacy will be evaluated in tumor bearing mice in vivo.

FIGS. 24A and 24B show rapid formation of DO-VAC vehicles. FIG. 24A is an agarose gel showing rod shaped-DO nanostructures constructed within 5 minutes. FIG. 24B contains TEM images showing rod-shaped DO-VAC vehicles formed effectively at 10 minutes and 2 hours.

FIGS. 25A to 25H show DOVAC formulation characterization and immune activation. FIG. 30A is an agarose gel electrophoresis showing colocalization of DOVAC (V=DOVAC) components and stability in 95% non-heated serum at 37° C. for 0 h-24 h. FIG. 30B is a TEM image showing precise and uniform DOVAC geometry. (C) TEM image showing Ab attached to DOVAC utilizing a medium number of attachment sites. FIG. 30D shows flow cytometry of antigen presentation and activation markers in DCs in vitro. FIG. 30E shows DOVAC enhanced delivery to DCs via APC targeting ligands in vitro. FIG. 30F shows flow cytometry of OVA-specific T cell (from OT-1 mice) expansion 3 days after their adoptive transfer in WT mice and mice S.C. injection of either buffer, SIINFEKL (SEQ ID NO:2) (OVA)+CpG mixture, or DOVAC. FIG. 30G is same as FIG. 30F but showing T cell activation marker. FIG. 30H shows flow cytometry assessment of target cell killing in vivo 7 days post S.C. injection of indicated conditions. Data shown as mean (% gated)±SEM and n3″.

FIG. 26 shows quantification of endotoxin levels in p7249 scaffold and vaccine adjuvant formulations.

FIGS. 27A to 27J shows comparison of antigen presentation and costimulatory molecule expression for the controls, DNA origami (DO64)-based vaccine, electrostatically bound mixture of CpG adjuvant and K10SIINFEKL (SEQ ID NO:1), branched nucleic acid structure (Br)-based vaccine, branched DNA origami vaccine, and their specific formulations in dendritic cell model cell culture in vitro using MUTUDC1940 cells.

FIGS. 28A to 28X show comparison of antigen presentation and costimulatory molecule expression for untreated (PBS) control, mixture of OVA peptide and CpG, and branched DNA origami vaccine in splenocyte cell culture in vitro in CD11c positive dendritic cells, CD11b positive macrophages, and CD19 positive B cells.

FIGS. 29A to 29J show extended comparison of antigen presentation and costimulatory molecule expression for various controls and vaccine formulations as indicated in splenocyte cell culture in vitro in CD11c positive dendritic cells.

FIGS. 30A to 30J show extended comparison of antigen presentation and costimulatory molecule expression for various controls and vaccine formulations as indicated in splenocyte cell culture in vitro in CD11b positive macrophages.

FIGS. 31A to 31J show extended comparison of antigen presentation and costimulatory molecule expression for various controls and vaccine formulations as indicated in splenocyte cell culture in vitro in CD19 positive B cells.

FIGS. 32A to 32G show vaccine T cell killing efficacy. Target (target antigen carrying cells) and non-target cells were differentially stained with Cell Trace Violet and injected in mice 7 days post vaccination to compare conditions as indicated.

FIG. 33 shows evaluation of controlling vaccine charge on vaccine T cell killing efficacy and verification of bivalent cancer vaccine T cell killing ability directed against gp100 and Trp2, both carried on each vaccine nanoparticle.

FIGS. 34A to 34C show tumor efficacy in C57BI6 mice inoculated with B16F10 melanoma cancer cells and treated or not with various vaccine formulation.

FIGS. 35A to 35C show K10-OVApept attachment to CpG/DO65.

FIG. 36A to 36D show attachment of LL37 to DNA nanostructures.

FIGS. 37A to 37C show evaluation of DO-VAC uptake by antigen presenting cells (APCs). FIG. 37A (images 1 and 2 left to right) shows THP1 monocytes and Mutu 19040 dendritic cells (DCs) cultured with FAM-labeled K10-OVA (SIINFEKL, SEQ ID NO:2) peptides overnight followed by fluorescence microscopy. FIG. 37A (images 3-5 left to right) shows Mutu 1940 DCs cultured with DO-VAC (Cy3-labeled) labeled with Atto647-CpG or (Atto647-CpG, Cy3-DO, and FAM-K10-OVA peptides) and imaged via fluorescence microscopy under fixed (images 3 and 4 left to right) or live conditions. FIG. 37A (image 6) shows murine splenic mononuclear cells were incubated with DO-VAC overnight followed by fluorescence microscopy. FIG. 37B shows Mutu 1940 DCs cultured with PBS, Atto647-CpG alone, DO-VAC labeled with Atto647-CpG overnight and imaged via confocal microscopy (DAPI, nuclei; Lysosomes, Dextran Red; Atto647-CpG). Images in FIGS. 37A-37B are representative of 3 independent experiments. FIG. 37C shows murine splenic mononuclear cells cultured with Buffer (PBS), Atto647-CpG+K10-OVA peptides, or DO-VAC (Atto647-CpG+K10-OVA peptides) overnight and evaluated via Flow Cytometry (CD11c+, DCs; CD11b+, macrophages (MACs), CD19+ B cells). Data, top, mean Atto647-CpG % Gated+SEM; bottom, median fluorescence intensity (MFI) Atto647-CpG+SEM representing 3 independent experiments. Stats, multiple student's t test, *, p <0.05.

FIG. 38 shows murine splenic mononuclear cells cultured overnight in the presence of LPS, DO-VAC with scrambled antigenic peptides (Scrambled DOVAC), OVA peptide (SIINFEKL, SEQ ID NO:2), K10-OVA peptide, CpG, p7249, DO alone, CpG/DO, OVA, CpG+OVA, CpG+OVA+DO (physical mixture), or DO-VAC (DO+CpG+OVA peptides conjugated) followed by OVA/MHCl surface presentation on CD11c+DCs, CD11b+Macrophages, and CD19+B cells. Data is expressed as mean (MFI)+SEM and represents 3 independent experiments. Stats, multiple student's t test, *, p <0.05.

FIGS. 39A and 39B show evaluation of DO-VAC mediated APC activation. FIG. 39A shows Mutu 1940 DCs cultured overnight in the presence of K10-OVA peptides, OVA+CpG, DO vehicle, DO+K10-OVA, or DO-VAC (DO+CpG+OVA peptides). CD40 (top) and CD80 (bottom) surface expression was evaluated via flow cytometry where data is expressed as mean (MFI)+SEM top or mean (MFI)+SEM representing 3 independent experiments. FIG. 39B shows murine splenic mononuclear cells were cultured overnight in the presence of OVA peptide, staple oligos, p7249, CpG, DO-VAC (conjugated DO+CpG+K10-OVA), LPS, DO, CpG+OVA (physical mixture), or CpG/DO followed by CD40 top left and right or CD80 bottom left and right surface expression on CD11c+ DCs, CD11b+ Macrophages, and CD19+ B cells. Data is expressed as mean (MFI)+SEM and represents 3 independent experiments. Stats, multiple student's t test, *, p <0.05.

FIG. 40 shows evaluation of a DNA origami (DO)-VAC platform in vivo. OT-1 OVA-antigen specific transgenic T cells were adoptively transferred into WT, C57/BI6 mice followed by immunization of Buffer, OVA/CFA, or DO-VAC (s.c.) for 24 hrs. Animals (2/group) were euthanized and regional draining lymph nodes were isolated and evaluated for CD69 and CD25 surfaces levels by flow cytometry.

FIGS. 41A and 41B show evaluation of DO-VAC efficacy in vivo. FIGS. 40A and 40B show animals (n=3/group) immunized with CFA/OVAK10, OVAK10+CpG, DO+OVAK10+CpG, or DO-VAC (s.c.). On day 7, Cell trace violet-labeled (low) C57/BI6 splenic mononuclear cells were pulsed with OVA peptide antigen, while Cell trace violet-labeled (high) were not. Both cell subsets were mixed and adoptively transferred into C57/Bl6 mice. Splenic mononuclear cells were collected 20 hr later and evaluated for viability by flow cytometry. FIG. 41A shows representative histograms and FIG. 41B shows mean % of Target cells Killed+SEM. Animals (n=3/group were immunized on days 0 and 7 with either CpG+OVA (physical mixture) or DO-VAC where data is expressed mean % of Target cells Killed+SEM. Stats, multiple student's t test. *, p<0.05.

FIG. 42 shows Mutu 1940 DCs cultured with either Buffer, DO-VAC, or Aptamer conjugated DO-VAC overnight followed by flow cytometry to monitor OVA/MHCl surface expression. Data, mean OVA/MHCl peptide MFI+SEM representing 2 independent experiments.

FIGS. 43A and 43B show DO-VAC Bio-distribution Profile. FIG. 42A shows albino C57/Bl6 mice immunized (i.m.) or (s.q.) (left side) with either DO-atto647-CpG or DO-atto647-CpG-OVA-K10 (n=2 mice/treatment group) and subjected to Live Animal Imaging (IVIS) on days 0, 1, and 3 post injection (620 nm excitation, 670 nm emission). FIG. 42B shows on day 3 post injection, animals were imaged, euthanized, and tissues (inguinal lymph nodes and spleens) were collected and imaged via IVIS. The data is expressed as Epi-Fluoresence (Radiant Efficiency (p/sec/cm2/sr)/(μW/cm2)). Representative mice and tissues from the same mice are shown. LN, lymph node; rt, right; If, left; SP, spleen.

FIG. 44 shows DO-VAC Bio-distribution Profile. Albino C57/Bl6 mice were immunized (i.m.) or (s.q.) (left side) with either DO-atto647-CpG or DO-atto647-CpG-OVA-K10 (n=2 mice/treatment group) and subjected to Live Animal Imaging (IVIS) on day 3 post injection (620 nm excitation, 670 nm emission). The data is expressed as Epi-Fluoresence (Radiant Efficiency (p/sec/cm2/sr)/(μW/cm2)). Representative mice are shown from a dorsal and ventral view.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

As used herein, the terms “optional” or “optionally” means that the subsequently described event or circumstance can or can not occur, and that the description includes instances where said event or circumstance occurs and instances where it does not.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a patient. A patient refers to a subject afflicted with a disease or disorder, such as, for example, cancer and/or aberrant cell growth. The term “patient” includes human and veterinary subjects. In an aspect, the subject has been diagnosed with a need for treatment for cancer and/or aberrant cell growth.

The terms “treating”, “treatment”, “therapy”, and “therapeutic treatment” as used herein refer to curative therapy, prophylactic therapy, or preventative therapy. As used herein, the terms refers to the medical management of a subject or a patient with the intent to cure, ameliorate, stabilize, or prevent a disease, pathological condition, or disorder, such as, for example, cancer or a tumor. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cancer, such as, for example, breast cancer, lung cancer, colorectal, liver cancer, or pancreatic cancer. In an aspect, cancer can be any cancer known to the art.

As used herein, the terms “administering” and “administration” refer to any method of providing a composition to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or peptide or pharmaceutical preparation and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, in an aspect, an effective amount of the polymeric nanoparticle is an amount that kills and/or inhibits the growth of cells without causing extraneous damage to surrounding non-cancerous cells. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “DNA origami” refers to nanoscale folding of DNA to create non-arbitrary two- and three-dimensional shapes at the nanoscale. DNA origami works by using a long “scaffold” strand of DNA and holding it together using short, 200-250 base, “staple strands.”

### DNA Origami Nanostructure

In some embodiments, the disclosed DNA origami nanostructure contains four internal cavities in order to maximize the surface area accessible to solution and allow for a larger cross-section for mechanical stability. Details on how to design and produce these nanostructures are provided in Halley P D, et al. Daunorubicin-Loaded DNA Origami Nanostructures Circumvent Drug-Resistance Mechanisms in a Leukemia Model. Small. 2016 12(3):308-20, which is incorporated by reference herein for these teachings. As shown in Halley P D, et al., staple sequences are included in specific locations within the DNA sequence (see Table S1 of Halley P D, et al.) so that the DNA scaffold folds into a rod-like nanostructure. DNA design software, such as caDNAno, can be used to design 3D DNA origami nanostructures as disclosed herein.

### Antigens

Peptide antigens are described, for example, in Abd-Aziz, N, et al. J Oncol. 2022 2022:9749363, and Buonaguro, et al. Vaccines (Basel). 2020 8(4):615, which are incorporated by reference in their entireties for the teaching of these antigens and their uses as vaccines. For example, in some embodiments, the antigen is a tumor antigen, such as HER-2, hTERT, mesothelin, MUC-1, p53, gp100, MART-1, PSA, PAP, tyrosinase, BAGE, MAGE, GAGE, PRAME, NY-ESO-1, EBV LMP-1/LMP-2A, HPV-E6/E7, HTLV-1, KRAS, NRAS, epitopes from BCR-ABL translocation, ETV6, NPM/ALK, or ALK. In some embodiments, the antigen is Nelipepimut-S (NP-S), MDX-1379 (gp100), MAGE-A3/NY-ESO-1, or G17DT.

In some embodiments, the tumor antigen is an antigen for acute lymphoblastic leukemia (ALL), Breast Cancer, Fibrolamellar hepatocellular carcinoma (HCC), Follicular Lymphoma, Gastric Cancers, Glioblastoma, hepatocellular carcinoma (HCC), Kidney Cancer, Lymphocytic Leukemia, Melanoma, non-small cell lung cancer (NSCLC), Ovarian Cancer, Pancreatic Cancer, Pediatric Brain Tumor, Prostate Cancer, small cell lung cancer (SCLC), smoldering plasma cell myeloma (SPCM), triple-negative breast carcinoma (TNBC), or urothelial/bladder cancer (UBC).

In some embodiments, the antigen is a human endogenous retroviral element (HERV). HERV-derived antigens have been used to develop cancer vaccines and chimeric antigen receptor (CAR)-expressing T cells, and can be adapted for use in the disclosed compositions and methods.

In some embodiments, the antigen is an mRNA antigen, such as a viral antigen. Therefore, in some embodiments, the antigen is a viral antigen. For example, in some embodiments, the virus is an influenza A, an influenza B, a cytomegalovirus (CMV), respiratory syncytial virus (RSV), coronavirus (e.g. SARS-CoV-2), human papillomavirus (HPV), varicella, dengue, diptheria, ebola, hepatitis, human immunodeficiency virus (HIV), encephalitis, measles, monkeypox, mumps, norovirus, polio, rabies, rotavirus, rubella, herpes, or zika virus.

Viral antigens are described, for example, in Pollard, A J, et al. Nature Reviews Immunology 2021 21:83-100, Kyriakidis, N C, et al. NPJ Vaccines. 2021 6(1):28; and Andrei, G, et al. Front. Virol., May 24, 2021, which are incorporated by reference in their entireties for the teaching of these viral antigens and uses in vaccines.

In some embodiments, the antigen is a β-amyloid (Aβ) or Tau peptide for production of an Alzheimer's Disease vaccine. Examples of peptide antigens are described in Malonis, R J, et al. Chem Rev. 2020 120(6):3210-3229, which is incorporated by reference in its entirety for the teaching of these peptides and their uses as a vaccine.

### Pharmaceutical Formulations

Also provided herein are pharmaceutical formulations that can include an amount of a DNA origami nanostructure described herein and a pharmaceutical carrier appropriate for administration to an individual in need thereof. The individual in need thereof can have or can be suspected of a cancer, a genetic disease or disorder, a viral, bacterial, fungal, and/or parasitic infection, or other disease or disorder in need of treatment or prevention. In some embodiments, the subject in need thereof is in need of a diagnostic procedure, such as an imaging procedure. The pharmaceutical formulations can include an amount of a disclosed DNA origami nanostructures, such as that can be effective to treat or prevent a cancer, a genetic disease or disorder, a viral, bacterial, fungal, and/or parasitic infection, or other disease or disorder or be effective to image the subject or a portion thereof.

Formulations can be administered via any suitable administration route. For example, the formulations (and/or compositions) can be administered to the subject in need thereof orally, intravenously, occularly, intraoccularly, intramuscularly, intravaginally, intraperitoneally, rectally, parenterally, topically, intranasally, or subcutaneously. Other suitable routes are described herein. In some embodiments, the disclosed DNA origami nanostructures contains an effective amount of a cargo molecule.

### Parenteral Formulations

The disclosed DNA origami nanostructures can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the disclosed DNA origami nanostructures can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric or nonionic surface active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the disclosed DNA origami nanostructures.

The formulation can be buffered to a pH of 3-8 for parenteral administration upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the disclosed DNA origami nanostructures in the required amount in the appropriate solvent or dispersion medium with one or more of the excipients listed above, as required, followed by filtered sterilization. Dispersions can be prepared by incorporating the various sterilized disclosed DNA origami nanostructures into a sterile vehicle which contains the basic dispersion medium and the required other ingredients from those listed above. Sterile powders for the preparation of sterile injectable solutions can be prepared by vacuum-drying and freeze-drying techniques, which yields a powder of the disclosed DNA origami nanostructures plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more disclosed DNA origami nanostructures. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nanoformulations for parenteral administration can also contain one or more excipients, such as dispersing agents, wetting agents, and suspending agents.

### Topical Formulations

The disclosed DNA origami nanostructures can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, the disclosed DNA origami nanostructures can be administered as a liquid formulation, such as a solution or suspension, a semi-solid formulation, such as a lotion or ointment, or a solid formulation. In some embodiments, the disclosed DNA origami nanostructures can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing a disclosed DNA origami nanostructures are also provided. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing a disclosed DNA origami nanostructures as described herein are also provided. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some embodiments of a cream formulation, the water-base percentage can be about 60% to about 75% and the oil-base can be about 20% to about 30% of the total, with the other percentages being the emulsifier agent, preservatives and additives for a total of 100%.

Ointments containing a disclosed DNA origami nanostructures as described herein and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

Also described herein are gels containing a disclosed DNA origami nanostructures as described herein, a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Also described herein are foams that can include a disclosed DNA origami nanostructures as described herein. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use.

Buffers can be used to control pH of a composition. The buffers can buffer the composition from a pH of about 4 to a pH of about 7.5, from a pH of about 4 to a pH of about 7, or from a pH of about 5 to a pH of about 7. In some embodiments, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations can be provided via continuous delivery of one or more formulations to a patient in need thereof. For topical applications, repeated application can be done or a patch can be used to provide continuous administration of the noscapine analogs over an extended period of time.

### Enteral Formulations

The disclosed DNA origami nanostructures can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing a disclosed DNA origami nanostructures can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing a disclosed DNA origami nanostructures can be prepared using one or more pharmaceutically acceptable excipients, including diluents, preservatives, binders, lubricants, disintegrators, swelling agents, fillers, stabilizers, and combinations thereof.

Delayed release dosage formulations containing a disclosed DNA origami nanostructures can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The formulations containing a disclosed DNA origami nanostructures can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers can be used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### Additional Active Agents

In some embodiments, an amount of one or more additional active agents are included in the pharmaceutical formulation containing a disclosed DNA origami nanostructures. Suitable additional active agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoireanti-histamines, anti-infectives, and chemotherapeutics (anti-cancer drugs). Other suitable additional active agents include, sensitizers (such as radiosensitizers). The disclosed DNA origami nanostructures can be used as a monotherapy or in combination with other active agents for treatment or prevention of a disease or disorder.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatoires include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzatihine and benzylpenicillin), phenoxymethylpenicillin, cloxacillin, flucoxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefiximine, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, azrewonam, tigemonam, nocardicin A, taboxinine, and beta-lactam), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, all-trans retinoic acid, and other anti-cancer agents listed elsewhere herein.

### Methods of Using DNA Origami Nanostructures

The disclosed DNA origami nanostructures can be used to deliver one or more cargo compounds to a subject in need thereof or a cell. In some embodiments, the disclosed DNA origami nanostructures can be used to deliver an RNA or DNA molecule for replacement gene/transcript therapy, deliver RNAi or similar RNA (e.g. microRNA) to a subject to specifically inhibit RNA transcripts to reduce gene expression of a specific gene or genes, deliver an imaging agent, delivering a small molecule drug, and/or deliver any other cargo compound that can be loaded in the disclosed DNA origami nanostructures. Thus, the disclosed DNA origami nanostructures can be used to deliver a treatment, prevention, and/or a diagnostic compound to a subject in need thereof.

The disclosed DNA origami nanostructures can be used in some cases to treat a subject with a cancer. The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some embodiments, where the disclosed DNA origami nanostructures include a photocleavable linker that is linking the targeting moiety and/or cargo compound the DNA origami nanostructures can be administered to the subject or population of cells. After administration, light can be applied to the region and/or population of cells in the subject in need thereof where treatment or prevention is needed to cause the release of the disclosed DNA origami nanostructures and/or cargo molecule.

The disclosed DNA origami nanostructures as provided herein can be administered to a subject in need thereof, cell, or population thereof. The subject in need thereof can have a cancer, genetic disease or disorder, a viral, bacterial, parasitic, and/or fungal infection, or any other disease or disorder that would benefit from an effective agent (such as a cargo compound described herein) being delivered. The amount delivered can be an effective amount of a DNA origami nanostructures provided herein. The subject in need thereof can be symptomatic or asymptomatic. In some embodiments, the DNA origami nanostructures provided herein can be co-administered with another active agent. It will be appreciated that co-administered can refer to an additional compound that is included in the formulation or provided in a dosage form separate from the DNA origami nanostructures or formulation thereof. The effective amount of the DNA origami nanostructures or formulation thereof, such as those described herein, can range from about 0.1 mg/kg to about 500 mg/kg. In some embodiments, the effective amount ranges from about 0.1 mg/kg to 10 mg/kg. In additional embodiments, the effective amount ranges from about 100 mg/kg. If further embodiments, the effective amount ranges from about 0.1 mg to about 1000 mg. In some embodiments, the effective amount can be about 500 mg to about 1000 mg.

Administration of the DNA origami nanostructures and formulations thereof can be systemic or localized. The compounds and formulations described herein can be administered to the subject in need thereof one or more times per day. In an embodiment, the compound(s) and/or formulation(s) thereof can be administered once daily. In some embodiments, the compound(s) and/or formulation(s) thereof can be administered given once daily. In another embodiment, the compound(s) and/or formulation(s) thereof can be administered is administered twice daily. In some embodiments, when administered, an effective amount of the compounds and/or formulations are administered to the subject in need thereof. The compound(s) and/or formulation(s) thereof can be administered one or more times per week. In some embodiments the compound(s) and/or formulation(s) thereof can be administered 1 day per week. In other embodiments, the compound(s) and/or formulation(s) thereof can be administered 2 to 7 days per week.

In some embodiments, the DNA origami nanostructures(s) and/or formulation(s) thereof, can be administered in a dosage form. The amount or effective amount of the compound(s) and/or formulation(s) thereof can be divided into multiple dosage forms. For example, the effective amount can be split into two dosage forms and the one dosage forms can be administered, for example, in the morning, and the second dosage form can be administered in the evening. Although the effective amount is given over two doses, in one day, the subject receives the effective amount. In some embodiments the effective amount is about 0.1 to about 1000 mg per day. The effective amount in a dosage form can range from about 0.1 mg/kg to about 1000 mg/kg. The dosage form can be formulated for oral, vaginal, intravenous, transdermal, subcutaneous, intraperitoneal, or intramuscular administration. Preparation of dosage forms for various administration routes are described elsewhere herein.

### EXAMPLE EMBODIMENTS

Embodiment 1. A vaccine device, comprising a DNA origami nanostructure formed from a plurality of scaffold strands and a plurality of staple strands assembled into a rod shape, wherein a peptide antigen is attached to the DNA of the nanostructure by electrostatic interaction.

Embodiment 2. The vaccine device of embodiment 1, wherein the peptide antigen comprises at least 5, 6, 7, 8, 9, or 10 contiguous positively charged amino acids.

Embodiment 3. The vaccine device of embodiment 2, wherein the at least 5, 6, 7, 8, 9, or 10 continuous positively charged amino acids are at the N-terminus of the peptide antigen.

Embodiment 4. The vaccine device of embodiment 2 or 3, wherein the positively charged amino acids are lysine or arginine amino acids.

Embodiment 5. The vaccine device of embodiment 2 or 3, wherein the peptide antigen comprises at least 10 contiguous lysine amino acids.

Embodiment 6. The vaccine device of embodiment 2 or 3, wherein the peptide antigen comprises at least 10 contiguous arginine amino acids.

Embodiment 7. The vaccine device of any one of embodiments 1 to 6, wherein at least 1,000 peptide antigens are attached to the DNA nanostructure.

Embodiment 8. The vaccine device of any one of embodiments 1 to 7, comprising at least 30, 35, 40, 45, 50, 60, 70, 80, 90, 100 peptides per nm2.

Embodiment 9. The vaccine device of any one of embodiments 1 to 8, comprising at least 50, 60, 70, 80, 90, 100 nM peptide.

Embodiment 10. The vaccine device of any one of embodiments 1 to 9, wherein each of the plurality of scaffold strands are 5,000 to 10,000 nucleotides in length,

Embodiment 11. The vaccine device of any one of embodiments 1 to 10, wherein each of the plurality of scaffold strands are derived from a virus.

Embodiment 12. The vaccine device of any one of embodiments 1 to 10, wherein each of the plurality of scaffold strands are derived from bacteriophage M13.

Embodiment 13. The vaccine device of any one of embodiments 1 to 12, wherein the peptide antigen comprises a viral antigen.

Embodiment 14. The vaccine device of any one of embodiments 1 to 13, wherein the peptide antigen comprises a tumor specific antigen and/or tumor associated antigen.

Embodiment 15. The vaccine device of any one of embodiments 1 to 13, wherein the DNA origami nanostructure comprises one or more first single stranded DNA oligonucleotide attachment arms configured to bind to a first complementary DNA oligonucleotide strands.

Embodiment 16. The vaccine device of any one of embodiments 1 to 15, wherein the DNA origami nanostructure is encapsulated in an alginate capsule.

Embodiment 17. A method for vaccinating a subject, comprising administering to the subject the vaccine device of any one of embodiments 1 to 15.

A number of embodiments of the invention have been described. Nevertheless, it will be understood that various modifications may be made without departing from the spirit and scope of the invention. Accordingly, other embodiments are within the scope of the following claims.

### EXAMPLES

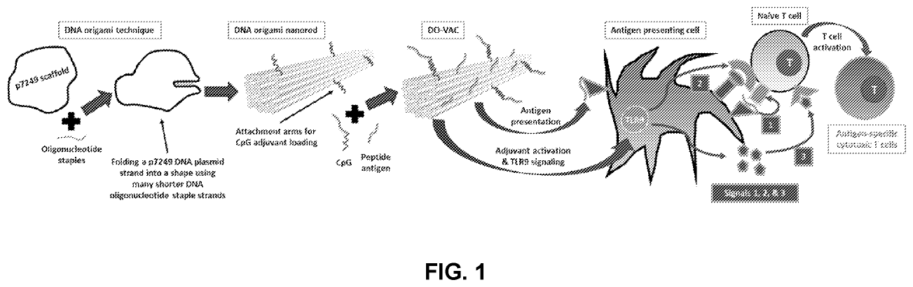

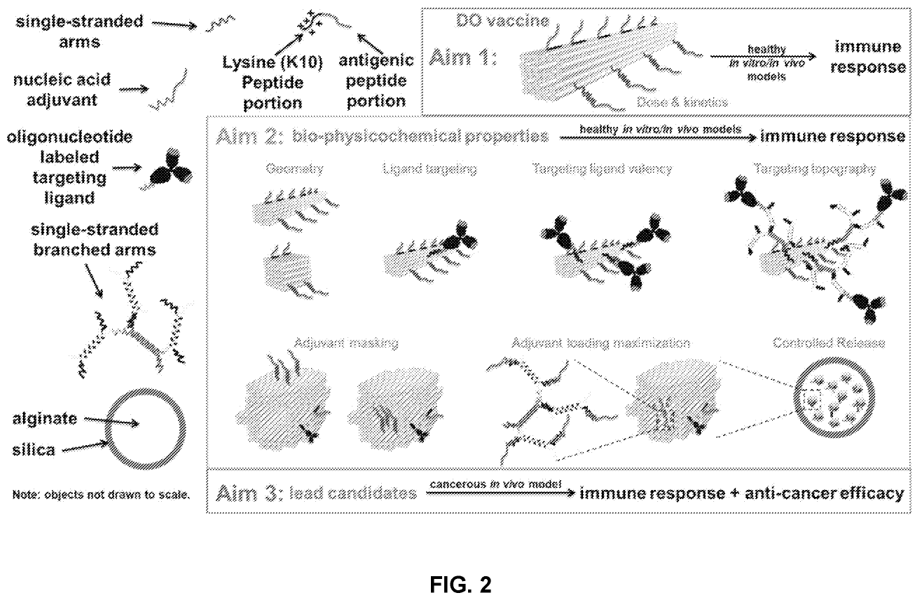

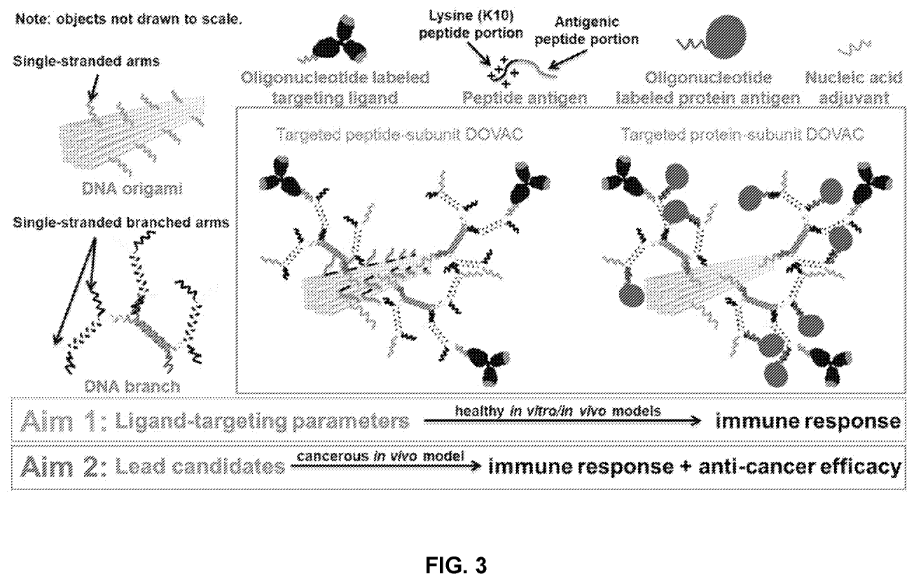

Nanoparticle (NP) geometry, surface organization, density, and types of molecules play one of the key roles in determining interactions of nanomedicines with biological environment, therefore affecting therapeutic index of nanomedicines. For example, NP immunorecognition, adjuvancy and antigenicity, biodistribution, clearance, pharmacokinetics and pharmacodynamics, cell binding, internalization, and intracellular fate are some of the biological processes that are affected by NP properties. This has been demonstrated for vaccine delivery, particularly regarding geometry dictating organization of antigens on nanoparticle surface. In this regard, nanoparticles have the ability to mimic pathogens, therefore enhancing adjuvancy and antigenicity of the molecules used in vaccines. DNA origami, a method to fold DNA strands into 2- and 3-dimensional geometries, provides unprecedented control over the overall construct geometry, but also geometry of surface molecules, including therapeutics, targeting ligands, protecting agents, etc. However, although DNA origami-based delivery methods have intrinsic ability for high capacity and loading efficiency of intercalating drugs, DNA origami has limited ability to load drugs (e.g., peptides, proteins, nucleic acids, small molecules, etc.) via conjugation due to the limited attachment sites. This Example describes DNA origami-based devices, methods, and new applications useful for enhancing or better controlling therapeutic index (i.e., therapeutic efficacy in relation to side effects) of vaccines, including all other nucleic acids, their modifications, and alternatives, and is applicable to many delivery and sensing applications using DNA nanoparticles (FIGS. 1-3).

### Example 1

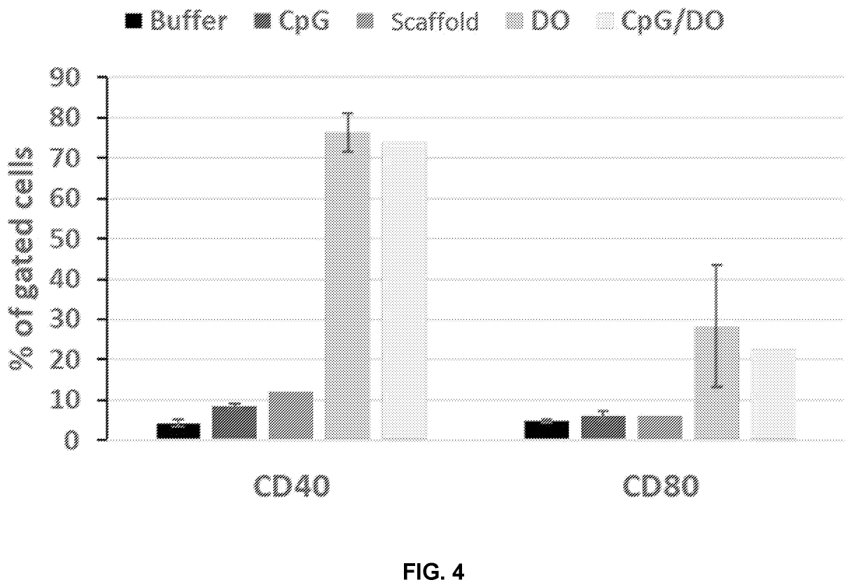

Disclosed herein is a vaccine adjuvant function for DNA origami (DO), wherein folding of M13mp18 bacteriophage single stranded genomic DNA (scaffold) into DNA origami allows DNA origami to act as an adjuvant with improved activity compared to unfolded scaffold DNA. Folding endotoxin free single stranded DNA scaffolds of viral origin into DNA origami structures represents a method to provide enhanced adjuvant properties as compared to the starting unfolded materials (scaffold DNA and oligonucleotide strands). This is unexpected finding since the state of the art in the field is to employ adjuvants by attaching adjuvant molecules such as CpG, etc., to the nanoparticles, including DNA origami. Hence, this represents a new class of adjuvants, which are defined as origami technique folded nucleic acid molecules, particularly sequences incorporating, but not limited to CpG. In vitro assays showed immune activation of dendritic cells (FIG. 4). The enhanced adjuvancy of DNA origami nanostructures as compared to scaffold and oligonucleotides by themselves may come from the increased cell uptake, resistance to degradation, and improved diffusion properties of folded scaffold (i.e., compact form) as compared to unfolded scaffold. Please note that even after DO assembly and increased immunogenicity, the immunogenicity is still appropriate, and no toxicity has been observed. CpG sequences are already used in general population immunizations, demonstrating feasibility of our novel adjuvant use in clinics.

### Example 2

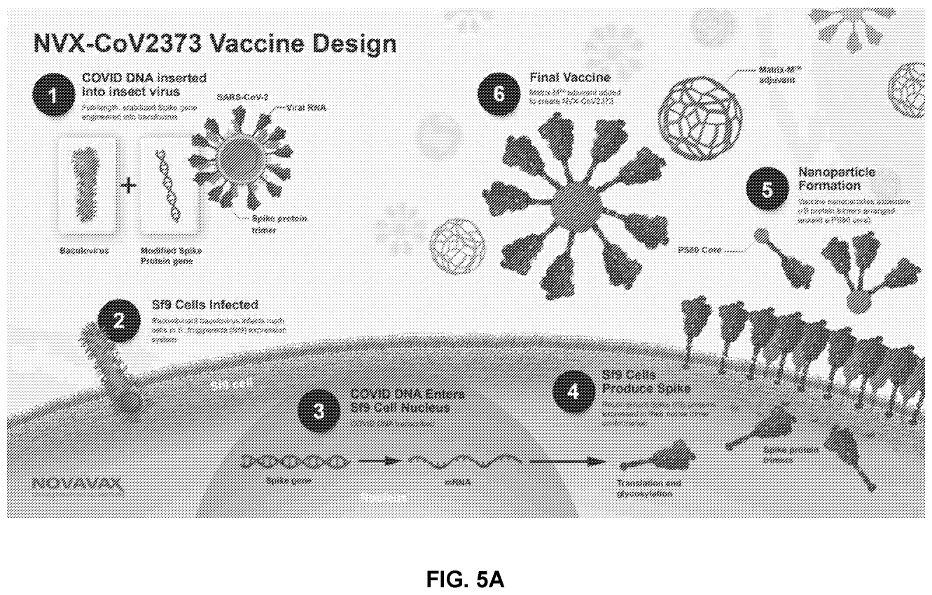

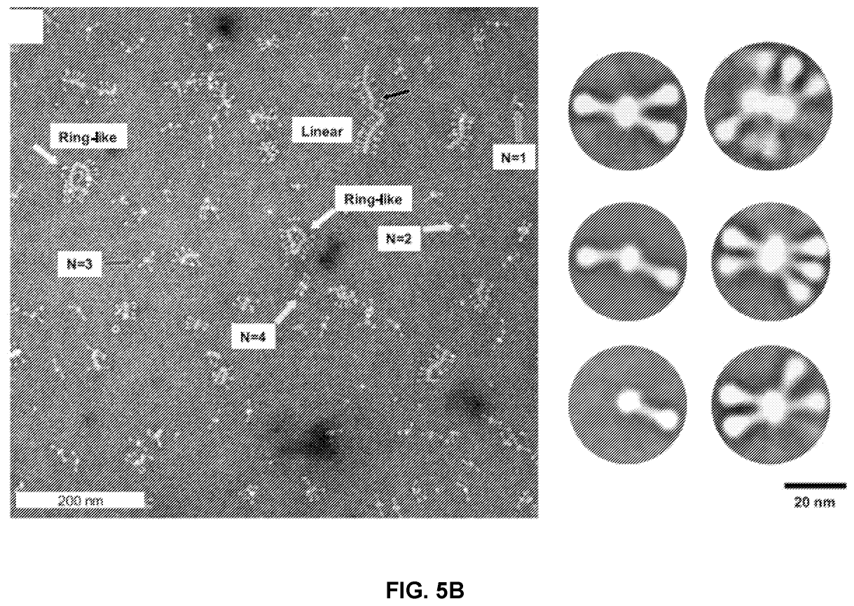

Also disclosed herein is a DO design that allows for potentially unlimited branching of DO attachment arms for ultrahigh payload capacity loading of DO. This maximizes surface density of attachment sites beyond any current existing DNA origami methods or designs. The density of antigens and targeting moieties on the nanoparticle surface are known to modulate immune responses and targeting. Currently, there are several vaccines in research that try to mimic the surface organization of antigens as in viral capsid proteins assembly to enhance vaccine efficacy (FIG. 5). In addition, viruses have evolved ligand-based targeting strategies, utilizing particular shapes and surface ligand organization and density. While this is being exploited for the vaccines using viral-like particle technology, the problem with this method is that it relies on protein self-assembly and incorporation of other molecules, such as targeting ligands is difficult and requires redesigning and optimizing manufacturing, whereas DNA origami provides advantage of modularity, where parts are assembled and interchangeable purely based on DNA sequence. DNA origami provides unprecedented control over the overall construct geometry, but also precise control of presentation of surface molecules on DNA origami via oligonucleotide protrusion arms on DNA origami surface (internal cavity or external cavity surface), including therapeutics, targeting ligands, protecting agents, etc.

Nevertheless, the number of oligonucleotide-based surface attachment sites is limited due to several aspects of DNA origami pertaining to the need to balance the number of attachment sites with: 1) the cost of the staple strands by keeping the strands as short as possible, and also 2) inherent disadvantage of introduction of mistakes as the strands get longer, 3) the stability of DNA origami or attachment arms itself, as higher number of attachment arms lead to shorter portions of these attachment oligonucleotide arms acting as staple segments in DNA origami, 4) potential aggregation problems with high attachment arm numbers. In addition, current methods do not offer attachment of molecules further away from the origami surface (for example 50 or 100 nm away from DO surface).

Although DNA branching has been previously utilized to make DNA nanoparticles, it has never been used to increase the density of attachment spots on DO or modification of DO surface. While DNA branched nanoparticles with high attachment density lack the ability of precise geometrical control and lack defined surface, DNA origami offers these advantages, but lacks high attachment sites. The utilization of a branched design is a new application of nucleic acid branches and DNA origami technique, where DNA origami is utilized to start and determine the starting surface topography and curvature (i.e., surface organization) of where branches start to propagate. This is in contrast to the standalone branches which are starting to branch from a very small point in space, which does not allow the maximum density of branching in the core due to the starting high surface curvature of such a small space, a phenomenon well known for a very small nanoparticles (e.g. 1-10 nm).

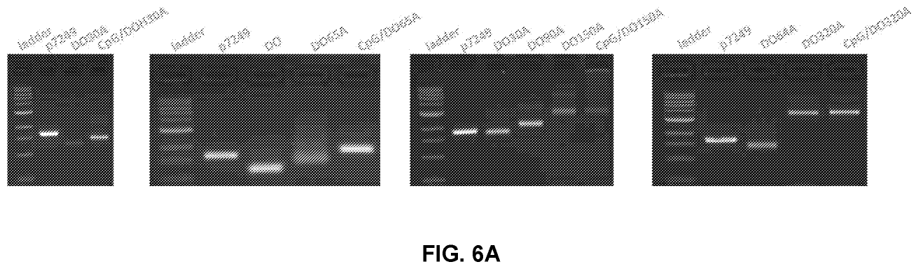

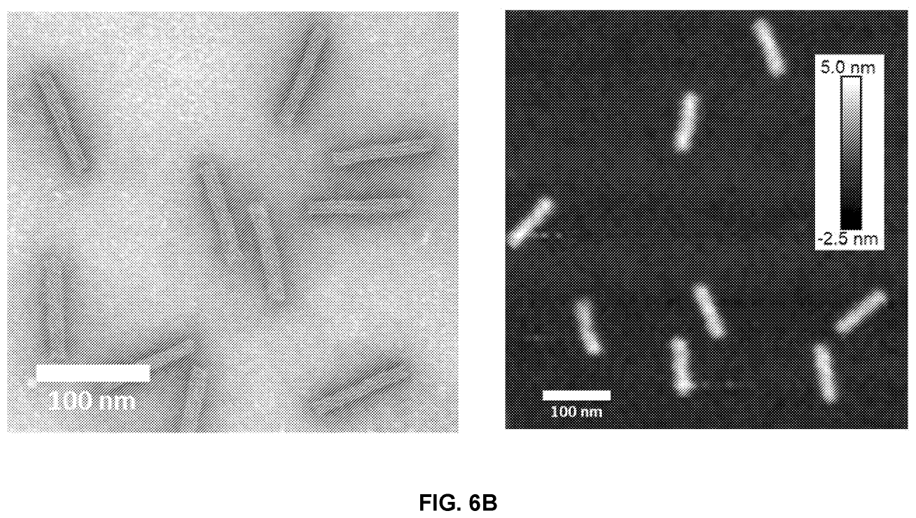

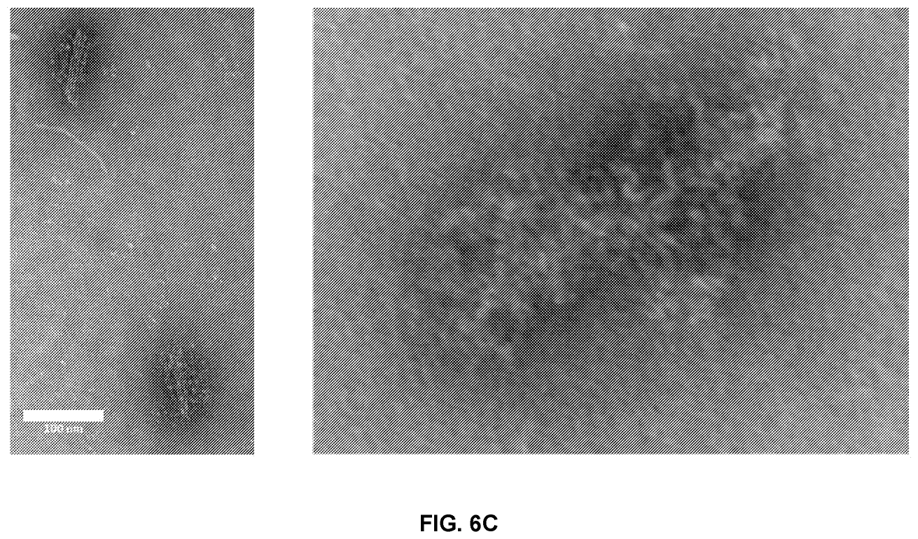

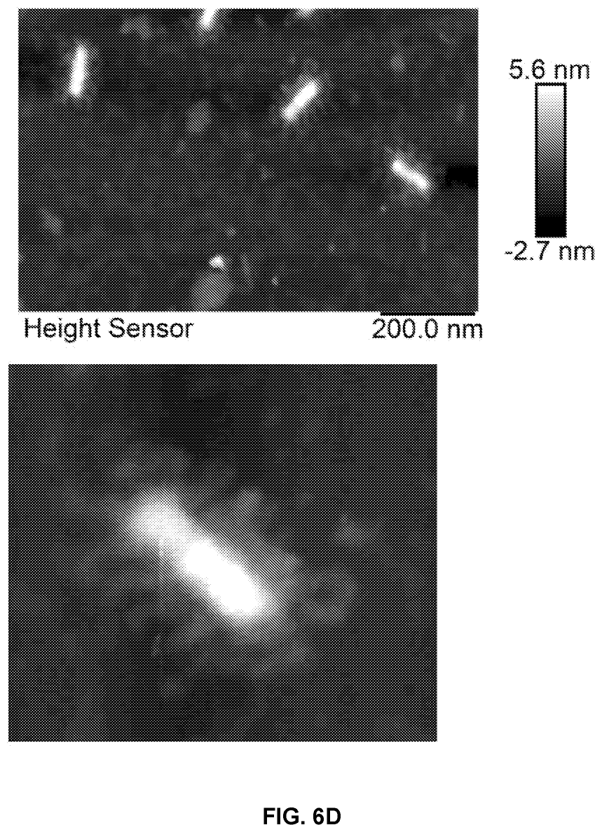

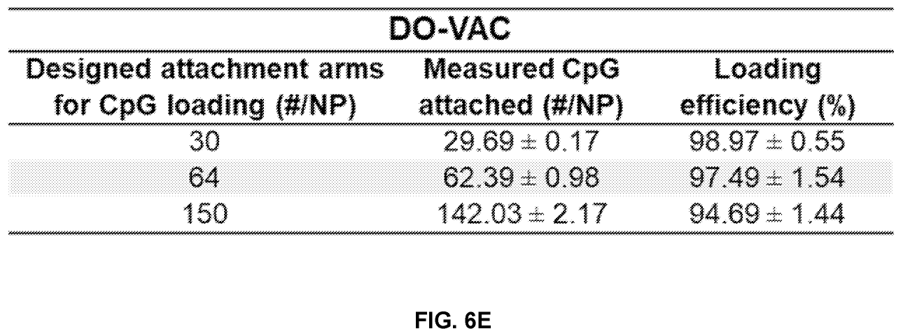

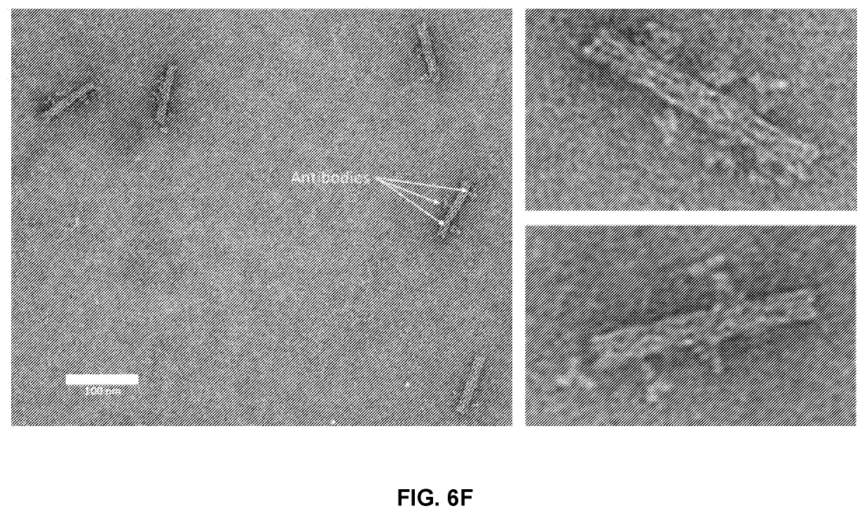

Also, the major difference due to the different starting point for branching is that high degree of precision of DO propagates into high degree of precision and high efficiency in branch self-assembly, allowing for 100% efficiency and branch assembly on the DNA origami surface and uniform branching based on a gel electrophoresis result. Described herein is a new nucleic acid-based branched assembly method to decorate origami surface with unlimited number of attachment sites and maximum density theoretically achievable. Disclosed is a DO design allowing attachment of over 300 or more therapeutic, sensing, and imaging molecules, including adjuvants, targeting ligands, etc, based on a new idea to utilize branched arms design and combine it with DO surface attachment to DO arms (FIG. 6).

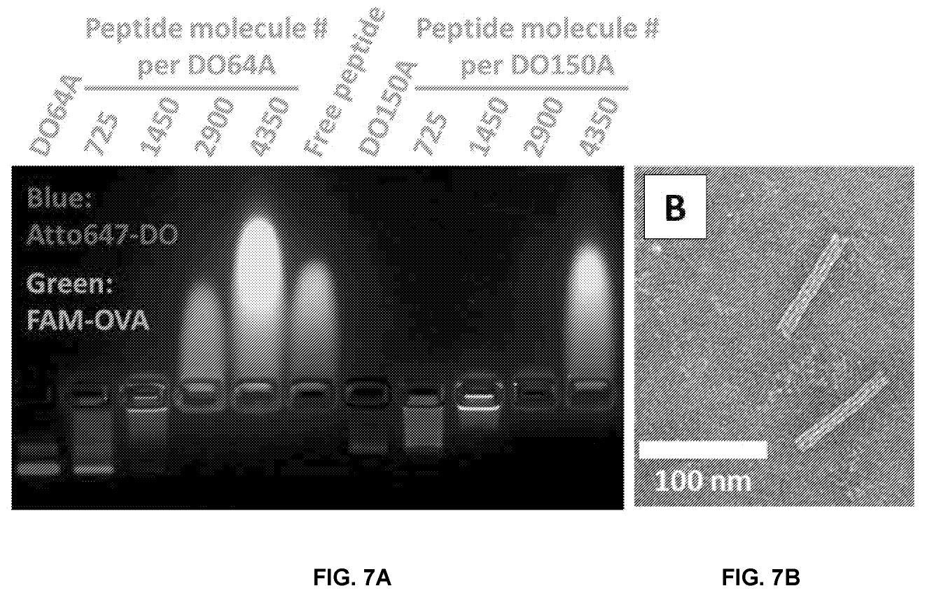

The unique branch sequences allow for one pot synthesis where all the components can be added at once in appropriate molar ratios. This is in contrast to current practices in the field where stepwise addition of each layer of oligonucleotides needs to be added. Other designs can utilize branches where branches themselves incorporate CpG sequences or other therapeutic sequences needed for particular applications, but efficiency and uniformity of such designs may be reduced. Currently, DO origami is limited in surface arm attachment number to less than 150 attachment arms per NP, and most designs have around 40 to 100 attachment spots; assembly has been confirmed up to 320 attachment arms. Furthermore, the topography of surface arms is limited as well, and is determined primarily by the DO geometry and constrained to a couple of tenths of nm from the origami surface. The disclosed method may be useful to allow for controlled topography away from DO surface, and utilization of 3D space on DO surface. In addition, DNA branching on DNA origami can presumably be used for modification of DNA origami nanoparticle surface flexibility as well as modulation of freedom of movement and thermal energy of attached ligands by controlling the length and the degree of branching of DNA branches attached to DNA origami. Surface character determines interactions with biological environment, for example protein adsorption, and even biofouling. This DNA branching method also allows for consequent growth in diameter and MW of DNA origami nanoparticles, resulting in increased capacity to load intercalating drugs as well as molecules that can interact with DNA, such as positively charged molecules (FIG. 7). Furthermore, as can be seen from AFM images, the disclosed surface modification results in visible attachment arm density on DO surface, not achievable by other methods (FIG. 6). Applications are numerous, from sensing, imaging, and even reducing the leakage of intercalating drugs from DO in circulation. The density of branched arms can be designed so high, that the layer of arms may be able to act as a sponge to slow down and control the release of chemotherapy drugs and other drugs. For this, the method where DO is first loaded with intercalating agents, DO containing intercalating agent can then be purified by pelleting, and lastly, high density DNA branches can then be self-assembled onto DO to provide a barrier layer with high affinity for intercalating agent such as that upon the leakage of intercalating agent from DO, intercalating agent is caught and intercalated by the network of DNA branches on the surface. This has application in for example reducing cardiotoxicity from chemotherapy based on intercalating agents such as doxorubicin. This is therefore a combination of DNA branch technology and DNA origami, and it has been discovered that structures can be made that are more efficiently assembled than found in the field.

### Example 3

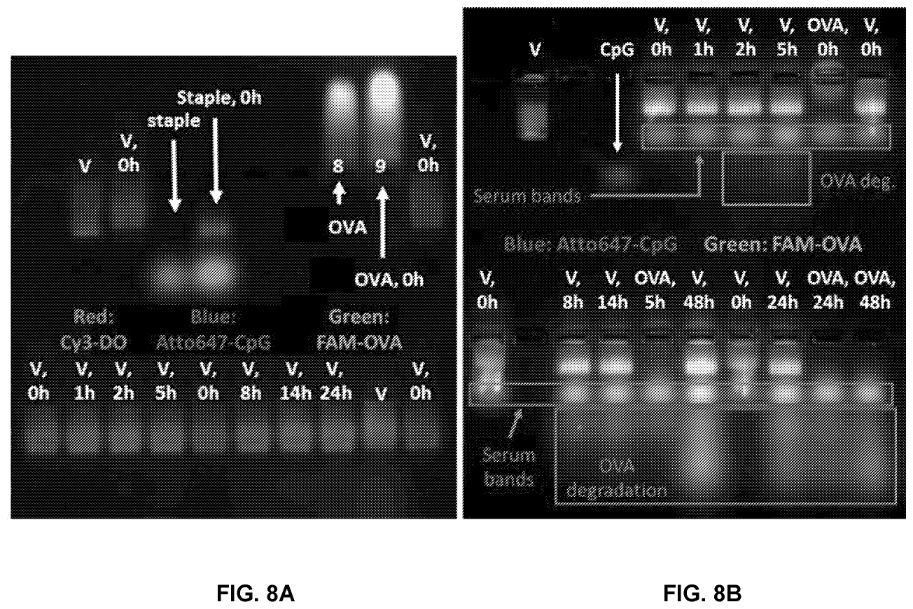

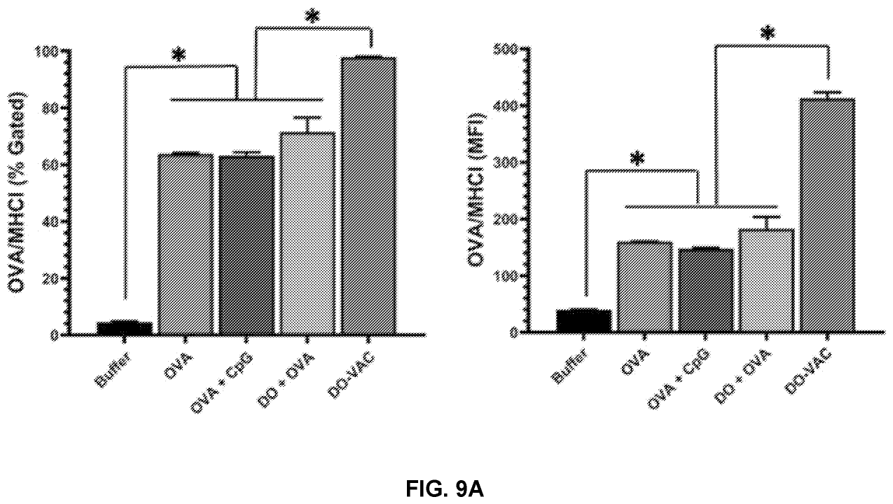

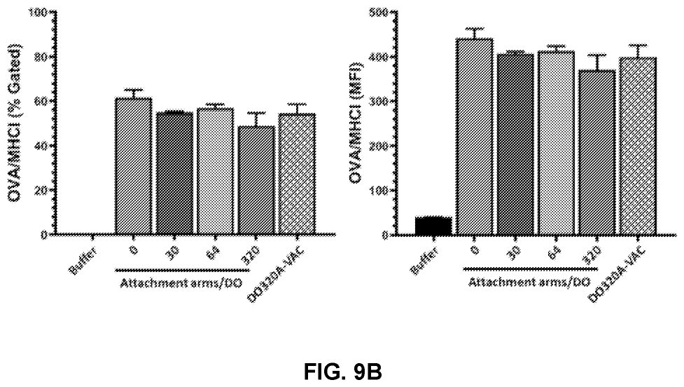

Also disclosed herein is a method for degradation resistant DO-based vaccine and a high DO loading capacity of peptides and proteins. DNA vaccine peptide loading technology was able to quickly induce presentation of peptide antigens to the MHC molecules on the surface of antigen presenting cells. Disclosed herein is unprecedented peptide antigen loading capacity, attachment efficiency, and surface density based on addition of several amine containing or positively charged amino acids to the native peptide (for example, 10 lysines or 10 arginines to the desired peptide sequence: KKKKKKKKKKSIINFEKL, SEQ ID NO:1, K10SIINFEKL). Some of these aspects may be translatable to protein loading on DO if proteins can be expressed with terminal peptide chains containing positive charges or amine groups. These amine containing amino acids, for example 10 lysines added to the OVA peptide (SIINFEKL, SEQ ID NO:2) sequence, facilitate electrostatic interactions to DO surface (FIG. 7), are resistant to degradation (FIG. 8), and ultimately result in rapid and highly efficient presentation of antigens on antigen presenting cells (FIG. 9).

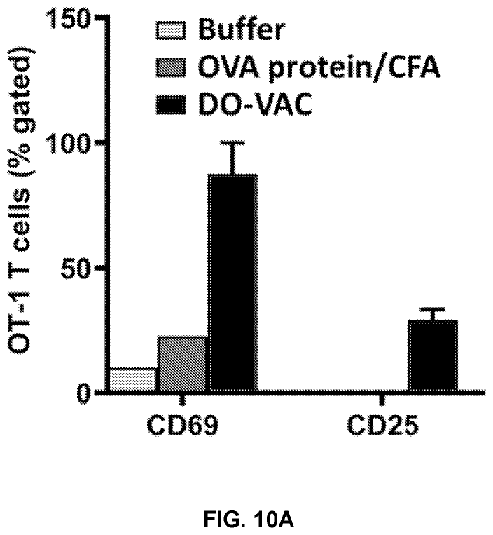

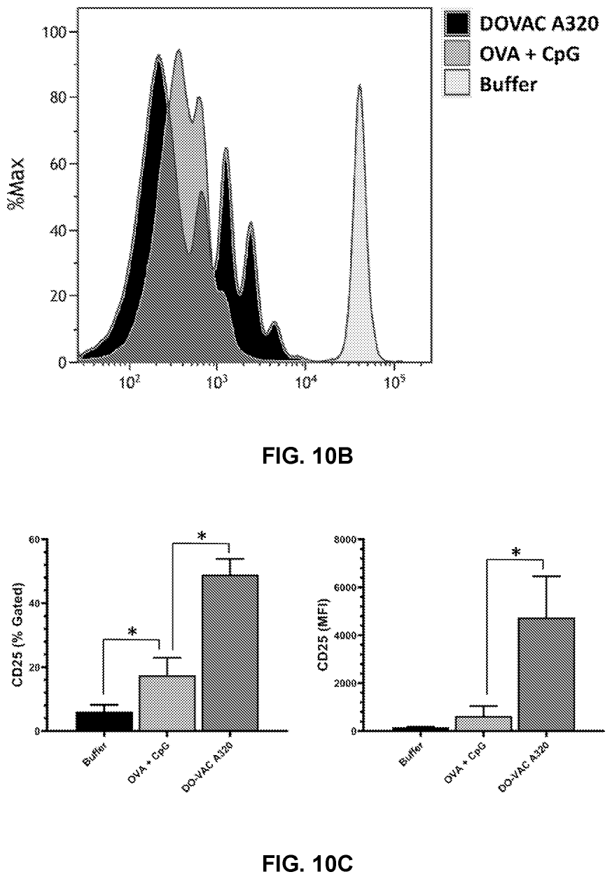

While previous method exists for complexing adjuvant nucleic acids and arginine-containing peptides, this process is not well controlled, and does not allow assembly of peptides on the surface of such constructs. The ability to place peptides exactly on the surface of nanoparticles, like in the DO design, may be important for rapid antigen processing due to accessibility to MHC molecules. This method describes for the first time a method for high efficiency peptide loading and possible utilization of the antigen peptide delivery mechanism where theoretically there may not be a need for nanoparticle internalization in order to achieve antigen presenting cell presentation of antigen peptides. This is because free peptides can directly bind to MHC on the cell surface. Importantly, this allows for excluding purification steps due to 100% attachment efficiency, and requires no additional modifications to the peptides except synthesizing peptides with additional positive charge containing amino acids. This design was confirmed in vitro and in vivo to demonstrate functional vaccine that is advantageous as compared to free adjuvants and antigens (FIGS. 10 & 11).

Also, this methodology can most likely be translated to protein attachment where proteins need to be expressed with one of the terminal ends containing K or R residues for electrostatic attachment to DNA origami or other negatively charged nanoparticles. Alternatively, proteins can be modified with K or R peptides post protein synthesis, but this will potentially result in multiple attachment spots on proteins instead the controlled one attachment spot possible during incorporation of positive residues during protein expression.

In some embodiments, proteins may be loaded onto DO by expressing DNA targeting antibody domain on native proteins to allow for binding of proteins to DO. Although many technologies have been described for DNA origami surface loading with peptides and proteins, such as oligonucleotide conjugation, none so far have the ability to cover the whole DNA origami surface with proteins and with such density as proposed herein. In terms of the protein loading, the disclosed method offers the same advantages as for the peptide loading. Although K10, a positively charged 10 lysine amino acids, have been used in the DNA origami filed to attach PEG to negatively charged DNA origami surface for the purposes of protection, this strategy has never been employed for peptide or protein antigen loading to attach up to 1500-3000 antigenic peptides or more on the single scaffolded DNA origami NP, depending on the design and size. Most importantly, the disclosed peptide attachment method preserves the vaccine activity of peptide and DO, which is not an obvious expectation from the previous literature. So far, a maximum of 20-40 peptide attachments on DNA origami vaccine has been reported. Furthermore, this kind of protective properties and prolonged antigen presentation was unpredictable. This is evidenced by attempts by others to hide these antigenic peptides within the cavity of DNA origami in order to protect the therapeutic antigens, hence unnecessary increasing the complexity and cost of the design, while limiting peptide antigen payload capacity of DNA origami. These data are at a very early time point of 30 minute post incubation with DO-VAC demonstrates high levels of antigen presentation. Antigenic peptides attached in such manner (i.e. on the surface of the DNA origami and exposed to potential degradation) are not only stable and protective to the DNA origami adjuvant, and DNA origami itself, but induce efficient and prolonged antigen presentation in dendritic cells as compared to the antigenic peptide by itself. In vitro degradation data show that antigenic peptides are protected in this manner as well when loaded onto DO. Lastly, it was discovered that PEG purification commonly employed for DNA origami via precipitation and pelleting does not work well or at all for the disclosed DO structures modified with peptides. This may be because of the surface modification that may make the structures behave differently, perhaps affecting their solubility, diffusion, prevent aggregation induced by PEG crowding, and potentially biodistribution properties in the body. This level of protection and new surface properties imparted on DO could ultimately have a monumental impact on DNA origami biomedical applications, including sensing in biological samples, and other nanoparticle delivery due to improved resistance of peptide modified structures to biofouling and interactions with other constituents of the bloodstream and other body fluids or tissues. It is known in the field that nanoparticles tend to aggregate in biological fluids, and this is one of the major hurdles and safety concerns.

### Example 4

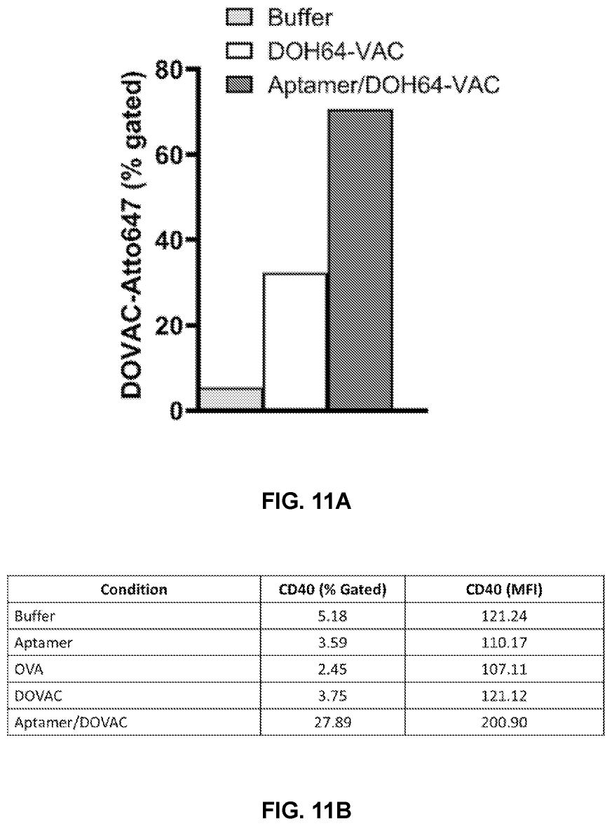

Also disclosed herein are DNA origami targeted to dendritic cells via ligands such as aptamers or antibodies to DEC 205 (FIG. 11). A portion of attachment region of a specific DEC205 aptamer was modified so that it can be produced commercially. However, the precise placement and high target aptamer density of the disclosed design is a key advantage. This is applicable to other targets found on APCs. No DNA origami has ever been shown to specifically to target dendritic cells, or other APCs so far. Tissue specific DO vaccines can be used to establish site-specific immunity by establishing resident memory T cells in a particular tissue of interest. While this has been achieved in lung tissue and a particular skin area by direct vaccine administration to lungs and a particular skin area respectively, no one has yet achieved this for most other organs. In this manner, specific sites can have protection from specific pathogens or cancer, particularly cancer metastasis. Branched DNA origami arm assembly can be included for improved targeting, particularly for lower affinity ligands such as aptamers. This design is envisioned to allow I.V. vaccine administration, while maintaining low side effects.

### Example 5

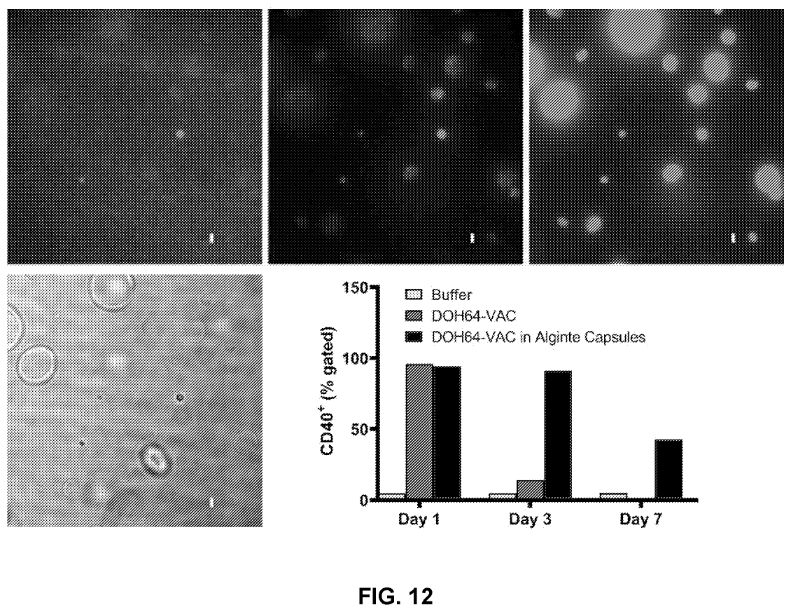

Also disclosed herein is a DNA origami-based capsule vaccine for controlled release and extended therapeutic activity. This version of the design utilizes gelatin, alginate, or other polymers capable of capsule formation to allow for the extended DNA origami vaccine activity at the injection site. This is not only useful for the traditional vaccine administration routes (e.g. S.C., I.M., oral), but also intranodal, and particularly important as potential cancer treatment route, intratumoral and peritumoral vaccinations (FIG. 12). In this particular case, conditions were optimized such that DOVAC was suitable for encapsulation into alginate capsules; air-blast nozzle technique and CaCl₂ (a common technique) were used to produce microcapsules, but methods exist for production of nanocapsules as well. The disclosed DO vaccine controlled release strategy based on alginate capsules does not require external force to induce immunization. In some embodiments, the method further involves stabilization and crosslinking strategies of peptides on DO surface. More importantly, results demonstrate stability of oligonucleotide attached molecules (CpG in this example), hence, peptides could in some embodiments be modified with oligonucleotides for attachment to DO. Since oligonucleotide-based attachment is a preferred method for protein attachment onto DO, this example indicates strong translational potential of this encapsulation strategy for protein antigen attachment to DO vaccine, particularly when combined with our branched DO design.

### Example 6

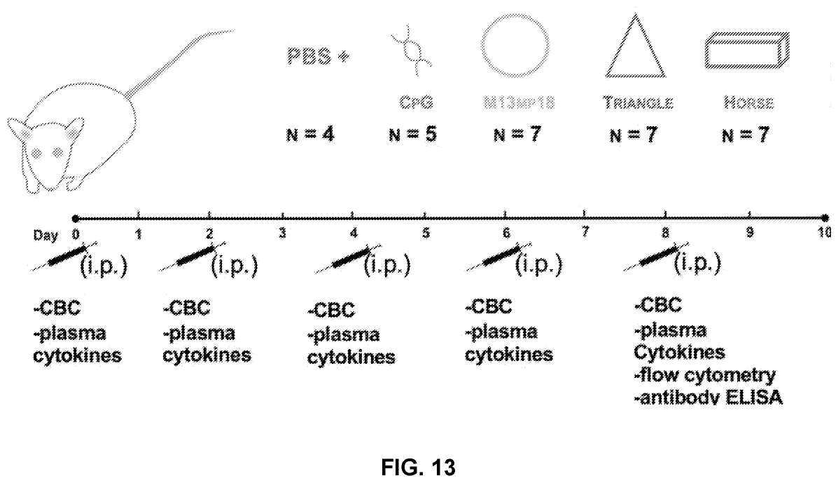

In order to determine whether DO nanostructures (flat 2D Triangle or 3D rod-shaped (Horse)) elicit immunogenicity and toxicity against a high dose (12.0 mg/kg) in vivo, a repeat (i.p.) dosing regimen was designed where complete blood counts (CBCs), plasma cytokine levels, flow cytometry, and antibody ELISAs were conducted at indicated time points (FIG. 13).

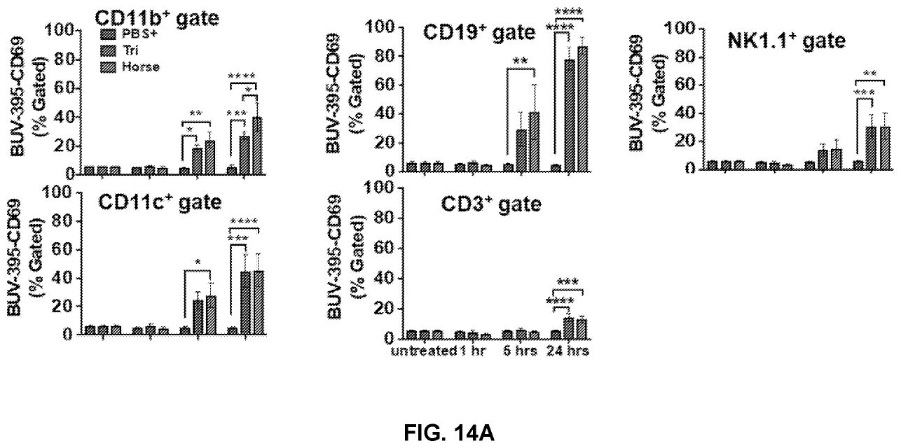

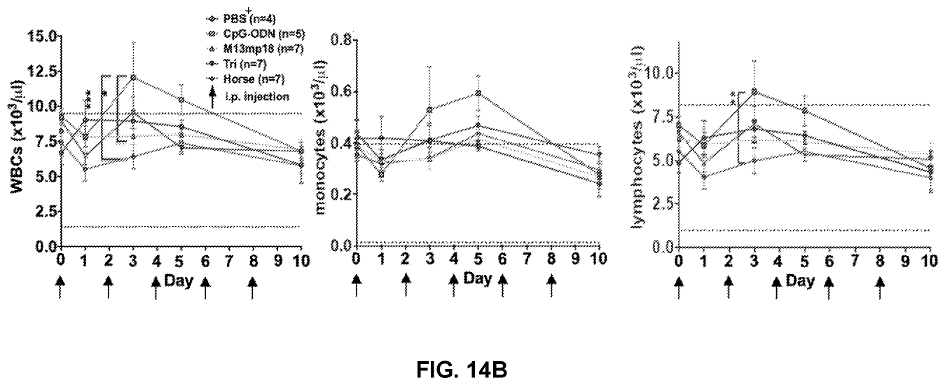

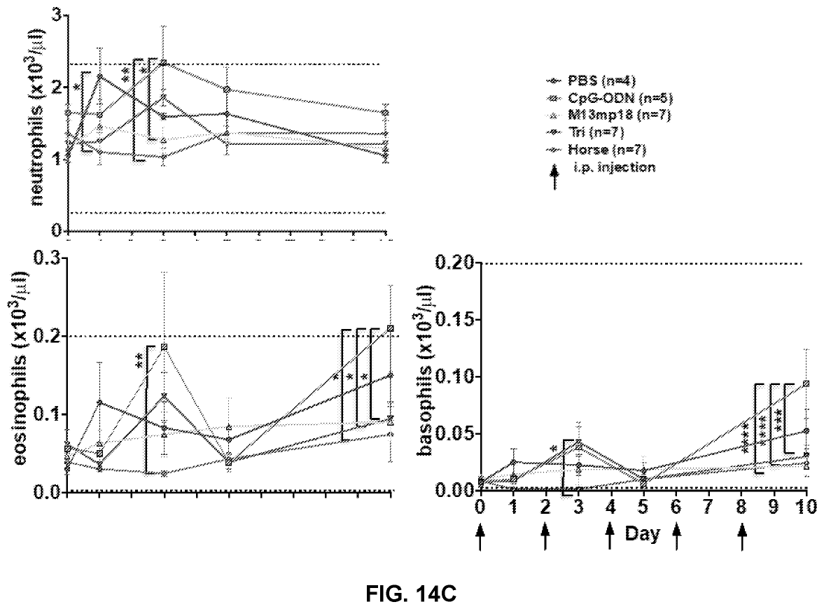

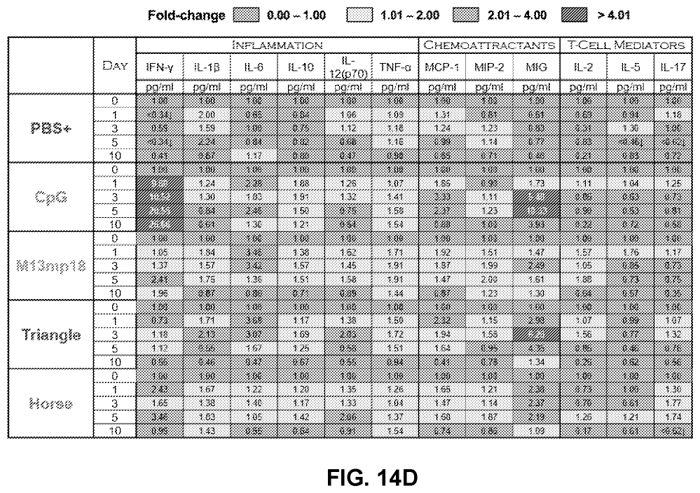

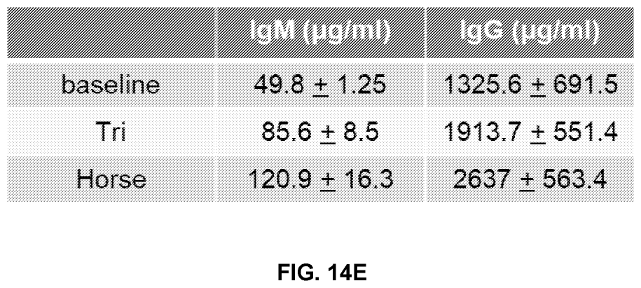

To evaluate if DO activates immune cells in vitro, the kinetics of immune activation was also tested at 100 nM DO concentrations. Tri and Horse DO induced significant time-dependent increases in CD69 relative to PBS treated controls for all cell subsets (FIG. 14A) with the greatest percent positive cell population being B cells followed by DCs, monocytes/macrophages, NK cells, and total T cells (FIG. 14A). While Tri and Horse DO increased the percentage of cells positive and levels of CD69 comparably for most cell types, Horse DO significantly increased the percentage of CD69⁺ monocytes/macrophages (CD11b⁺) relative to Tri DO at 24 hrs (FIG. 14A). This is consistent with the stronger localization of the Horse structures in these cells (FIG. 14A). Although Tri and Horse DO localized to the highest level in monocytes/macrophages, the strongest early immune activation via CD69 elevation occurred in the B cell population. With respect to CD69 elevation kinetics across all cell types, increases were observed beginning at 5 hrs and increased further up to 24 hrs. An exception was observed in T cells, which did not show an activation response until 24 hr (FIG. 14A). This is logical since DO localization increases were observed beginning at 1 hr post DO treatment (FIG. 14A) allowing for cellular recognition mechanisms to trigger gene expression and surface elevation of CD69 indicating early immune activation.

Potential DO immunogenicity in vivo (dosing described in FIG. 13) was evaluated by CBCs where CpG-treated mice showed elevated levels of WBCs, monocytes, lymphocytes, and eosinophils (FIGS. 14B and 14C), while Tri and Horse DO showed elevated monocytes (FIG. 14B). In order to evaluate immunogenicity at the molecular level, a panel of pro-inflammatory cytokines, chemokines, and T-cell mediators was evaluated by the Milliplex Cytokine/Chemokine array (FIG. 14D). Significant elevations of IFN-γ and MIG were observed among CpG-ODN treated mice, while a similar trend was evident among p7249, Tri, and Horse DO, although the response was moderate. Interestingly, both Tri and Horse DO cytokine profiles were distinct from CpG and p7249. The Tri DO response more closely resembled the p7249 response, which could be due to faster degradation in biological media as suggested by stability tests in FBS (FIG. 14D). Importantly, many elevated factors from p7249, Tri, and Horse DO treated mice decreased to at or below day 0 baseline levels by day 10 (i.e. no significant increases on day 10 relative to day 0). On days 0 and 10 (end point), plasma IgM and IgG levels were determined by ELISA (FIG. 14E), where both Tri and Horse DO showed elevated IgM and IgG at day 10 relative to baseline.

As an immune stimulant control, CpG-ODN was cultured with peripheral blood for 6 hours and analyzed by flow cytometry for CD69 levels where elevated was observed across all immune cell subtypes (FIG. 15).

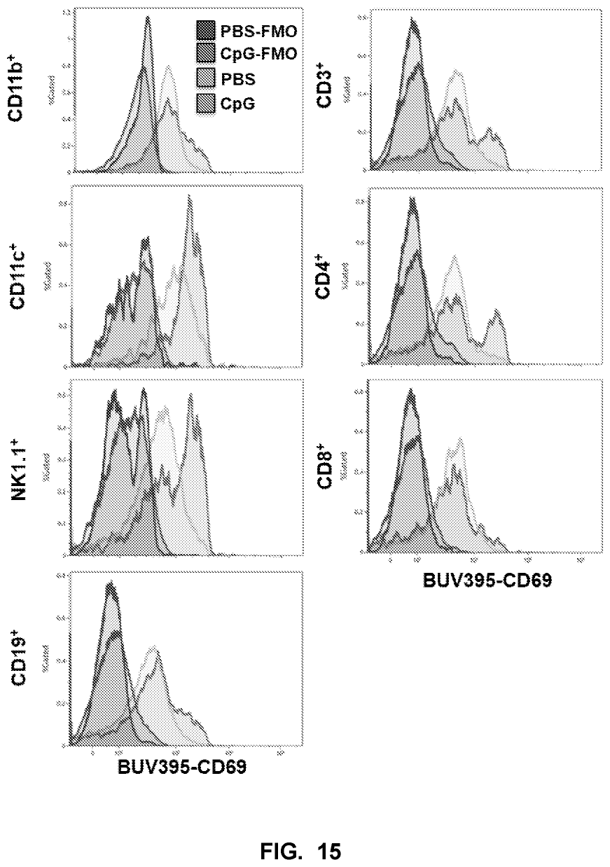

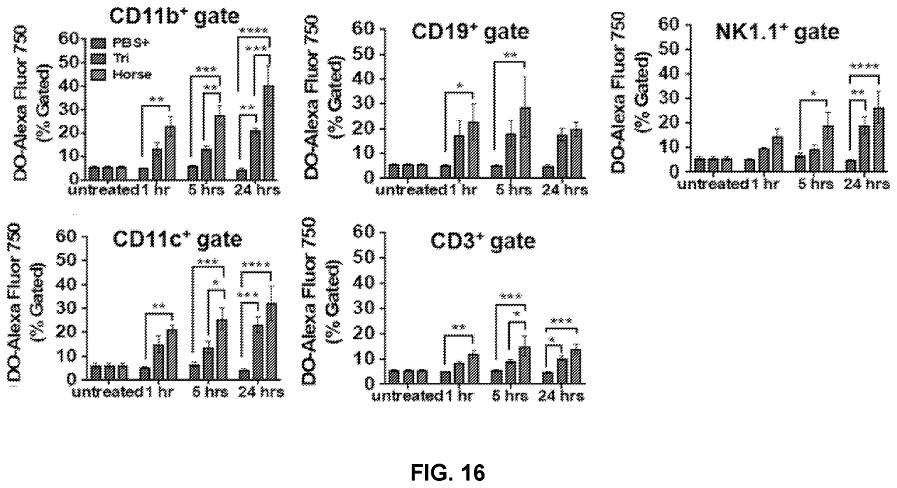

Cellular localization kinetics at 100 nM DO concentration were also investigated via flow cytometry at 1, 5, and 24 hr time points, which revealed significant time-dependent increases in localization of Tri and Horse DO in cell types up to 5 hrs (CD19+ (B cells), CD3⁺ (T cells), CD4⁺ T cells) and up to 24 hrs (CD11b⁺ (monocytes/macrophages), CD11c⁺ (dendritic cells), NK1.1 (NK cells), and CD8⁺ T cells) (FIG. 16). At 24 hrs, the CD11b⁺ (monocytes/macrophages), CD11c⁺ (dendritic cells (DCs)), NK1.1⁺ (natural killer (NK) cells), and CD19⁺ (B cells) cell populations all showed similar levels (% Gated) of DO localization, and CD3⁺ (T cells) cells showed lower amounts of localization. Horse DO was internalized to a significantly greater extent compared to Tri DO among the CD11b⁺ (at 5 and 24 hr) and CD11c⁺ (at 5 hrs) cell populations (FIG. 16).

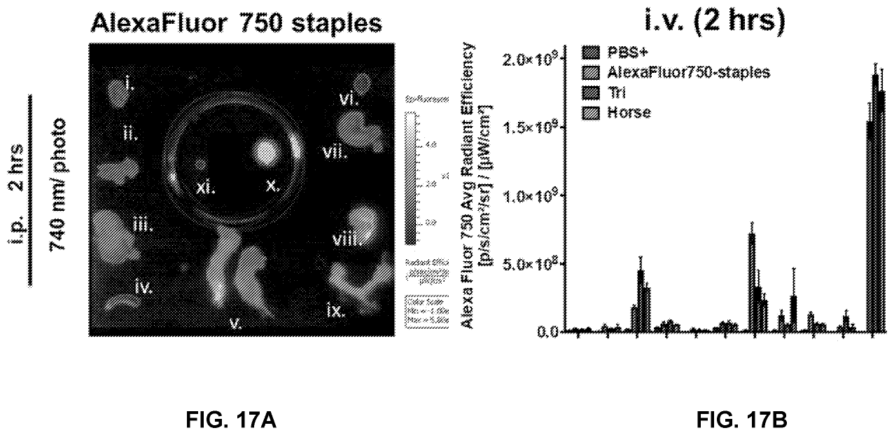

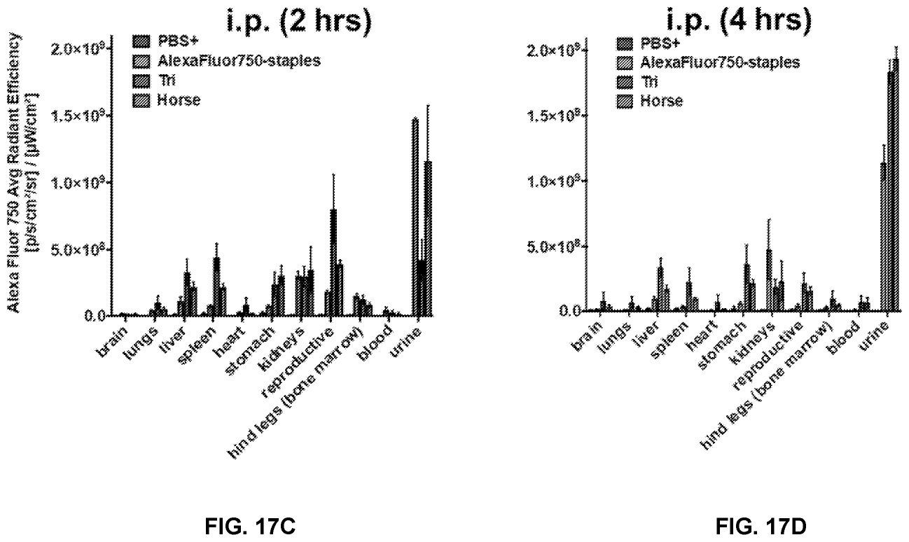

To evaluate biodistribution of Tri and Horse DO, mice were immunized with AF750-labeled oligonucleotides, Tri, or Horse DO either i.v. (for 2 hrs) or i.p. (for 2 or 4 hrs) then euthanized and major organs, blood, and urine were collected and imaged on an IVIS system to elucidate biodistribution profiles (FIG. 17A-17D). At 2 hrs post i.v. injection, the Alexa750-labeled Tri and Horse DO were localized mainly to the urine, kidneys, and the liver with the majority of signal observed in the urine (FIG. 17B). At 2 hrs post i.p. injection, the highest amount of Alexa750 oligonucleotides, and AlexaFluor750-labeled Tri and Horse DO were observed in the urine, reproductive organs, kidneys, stomach, spleen, and liver and no observable signal was detected in the blood and brain (FIG. 17C). At 4 hrs post i.p. injection, the highest Alexa750 signal was observed in the urine (increased relative to 2 hrs), kidneys, stomach, liver, spleen, and reproductive organs (all decreased relative to 2 hrs) (FIG. 17D).

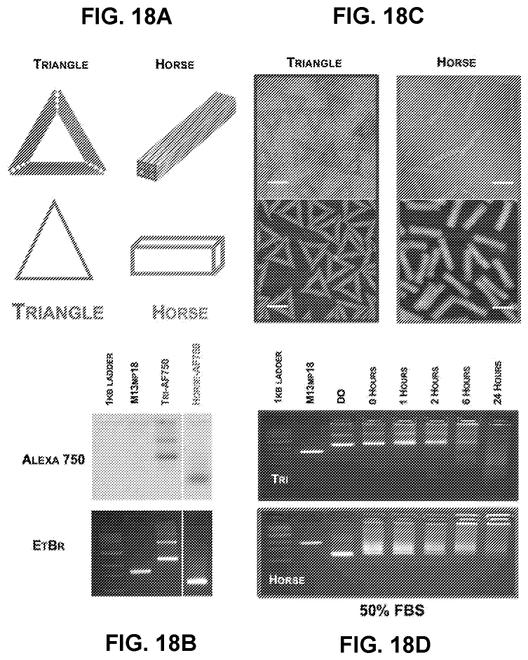

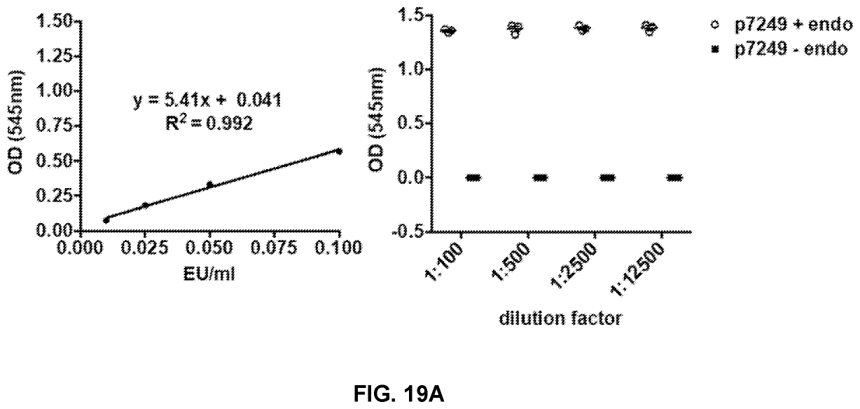

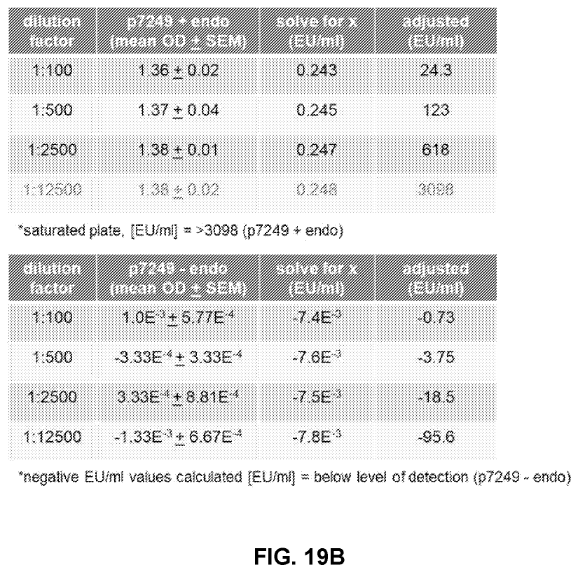

A flat single-layer 2D Triangle (Tri), first described (Rothemund, P W K, et al. Nature. 2006 440(7082):297-302) and a 3D rod-shaped ‘Horse’ DO previously described (Halley, P D, et al. Small. 2016 12(3):308-20) (FIG. 18A). Both DOs were fabricated in thermal annealing reactions scaled up to large volumes (20-80 mls) (Halley, P D, et al. Nano Research 2019 12:1207-1215) with scaffold material devoid of endotoxin, FIG. 19). Each structure was folded with four Alexa750-labeled oligonucleotides to enable live animal imaging. Agarose gel electrophoresis revealed well-folded Tri and Horse and proper incorporation of Alexa750-labeled oligonucletide (FIG. 18B). Leading bands were excised and imaged by transmission electron microscopy (TEM) and atomic force microscopy (AFM) to confirm proper folding (FIG. 18C). DO in vitro stability was evaluated in 50% FBS (non-heat inactivated) incubated at 37° C. Time-dependent decreases in structural integrity were observed in Tri, which were mostly degraded by 24 hours (hrs) as indicated by clear degradation products migrating faster on the gel (FIG. 18D). Horse DO remained significantly intact, although aggregation was observed in the gel wells at 6 and 24 hrs (FIG. 18D), suggesting the compact DO nanorod displayed enhanced stability compared to a flat DO triangle.

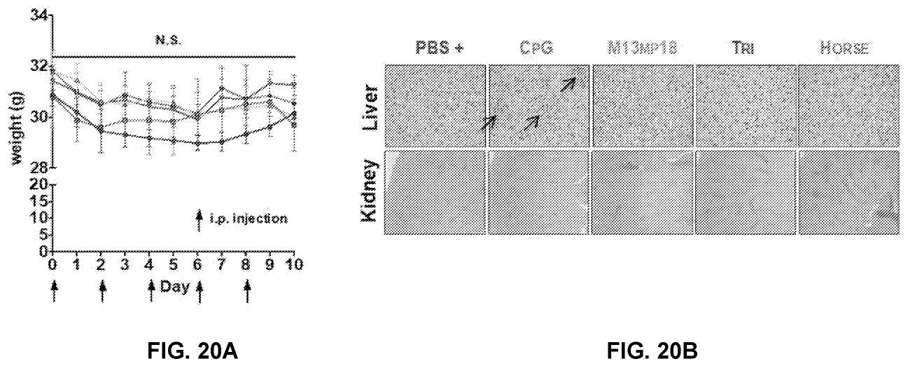

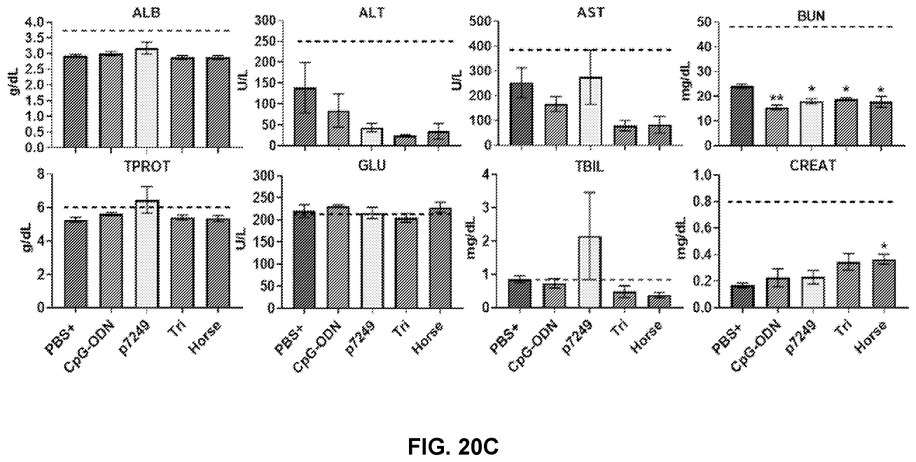

During the course of the repeat dosing regimen (described in FIG. 13), each animal was weighed daily to monitor overall health and potential toxicity. A transient decrease in weight (≤5%) among mice from all treatment groups out to day 3 was evident followed by stabilization in weight (FIG. 20A); however, no statistically significant differences were observed among treatment groups relative to the PBS control group.

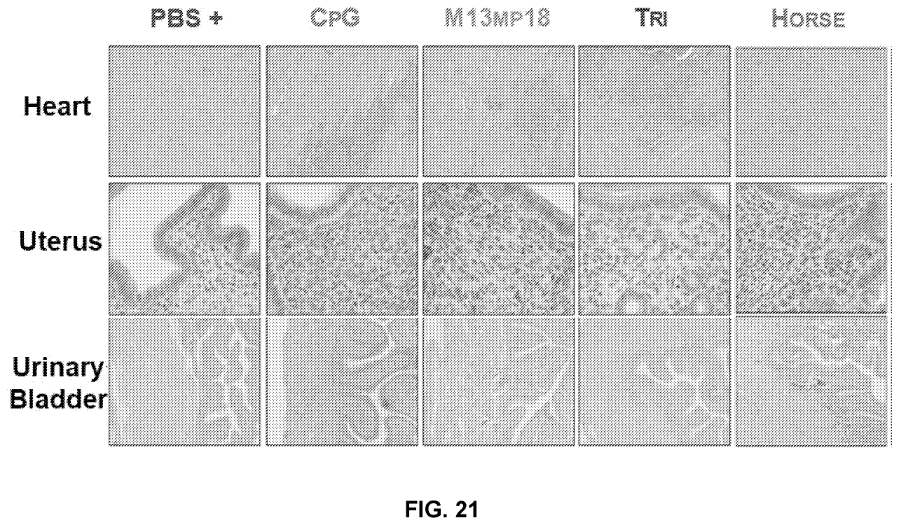

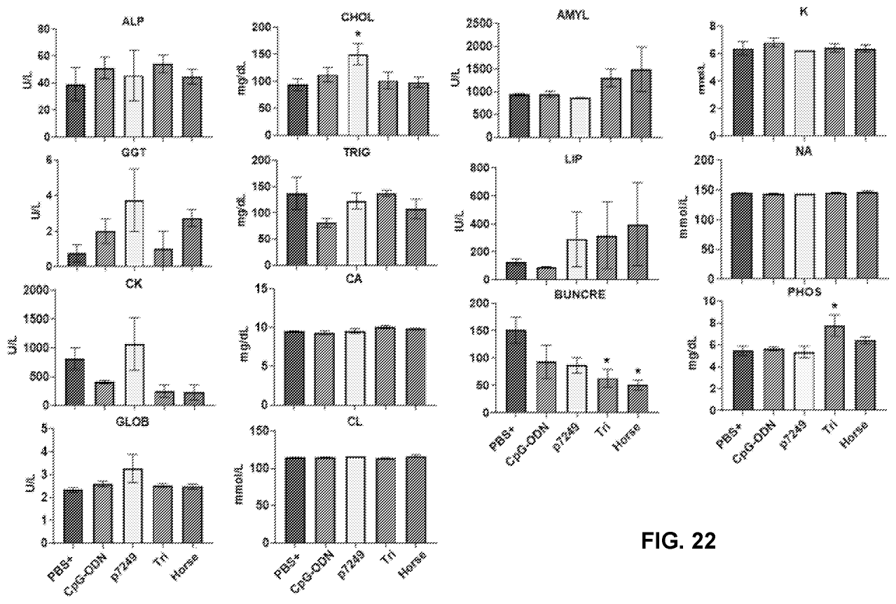

On day 10 (end point), whole mice were fixed in 10% neutral buffered formalin for histopathology evaluation. Representative images from H&E stained tissue sections from the liver and kidney are shown in (FIG. 20B), where mild hepatic necrosis lesions are identified by arrows in the liver sections from mice treated with CpG-ODN. Tissue sections from the heart, uterus, and urinary bladder sections are shown in (FIG. 21). Additionally on day 10, serum was analyzed via a Comprehensive Biochemical Panel (FIG. 20C and FIG. 22). All DO treatment groups displayed mean values of biochemical parameters either at or below the upper normal bound (Long, C T. et al. Lab Anim (NY). 2009 38(2):49-51; Wolford, S T, et al. J Toxicol Environ Health. 1986 18(2):161-88) with no significant difference between groups. The free p7249 scaffold (M13mp18) treated mice showing elevated levels of TBIL, suggesting potential liver effects of the unstructured ssDNA scaffold (FIG. 20C). However, all treatment groups reduced ALT levels relative to PBS+ treated mice, although the difference was not statistically significant and low levels typically indicate healthy liver function, since elevated ALT levels suggest liver injury (Toita, R, et al. J Toxicol Pathol. 2018 31(1):43-47) (FIG. 20C). All treatment groups decreased levels of BUN, where CpG-ODN, p7249 (M13mp18), and Horse were significant relative to PBS+ treated mice albeit a non-toxic result, while CREAT levels remained unchanged (FIG. 20C), likely indicating healthy kidney function (Keppler, A, et al. Kidney Int. 2007 71(1):74-8; Dunn, S R, et al. Kidney Int. 2004 65(5):1959-67; Metzger, C E, et al. PLOS One. 2021 23; 16(4):e0250438). Taken together, these multiple lines of toxicological evidence (weight, histopathology, biochemical panel, and lack of cytokine storm) indicate that Tri and Horse DO nanostructure treatments alone in vivo are non-toxic in response to repeat dosing at a high dose of 12.0 mg/kg.

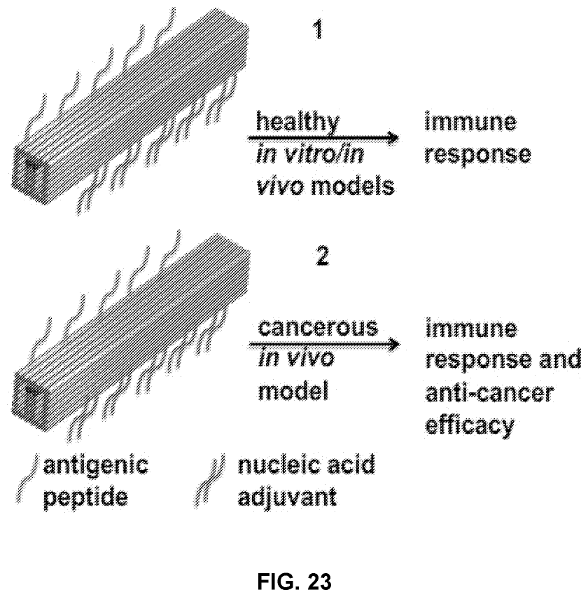

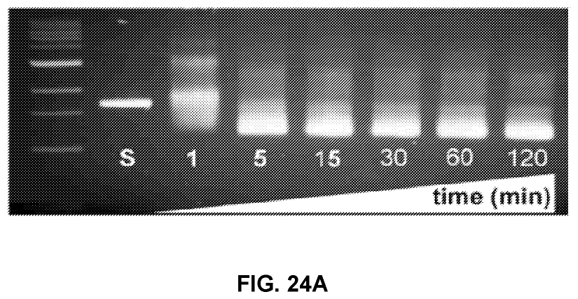

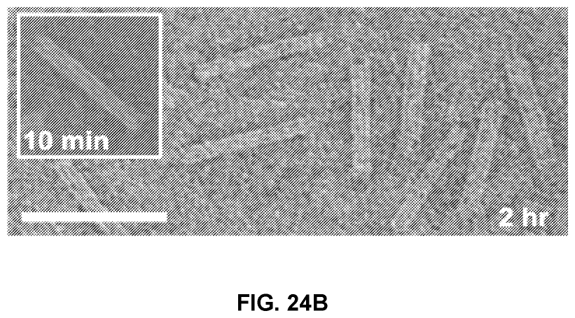

As FIG. 23 shows, this is a proof-of-concept approach. FIG. 24 shows rapid formation of DO-VAC vehicles. Agarose gel showing rod shaped-DO nanostructures constructed within 5 minutes (FIG. 24A). TEM images showing rod-shaped DO-VAC vehicles formed effectively at 10 minutes and 2 hours (FIG. 24B).

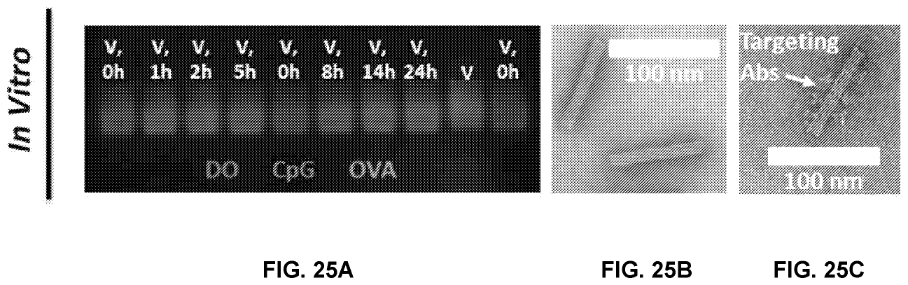

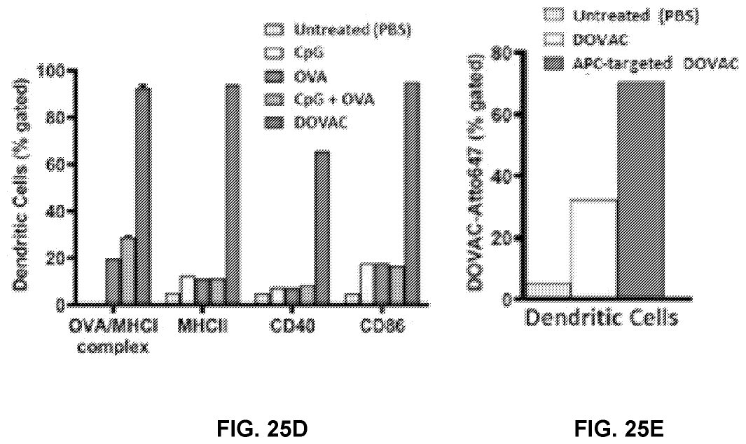

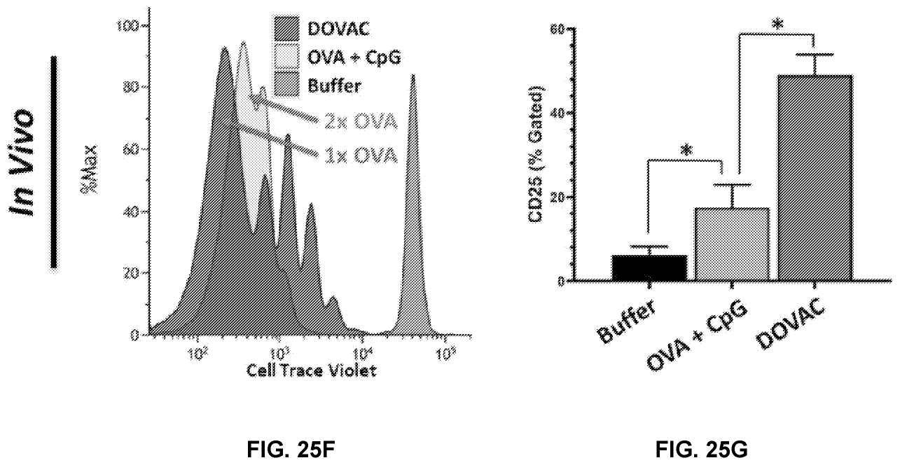

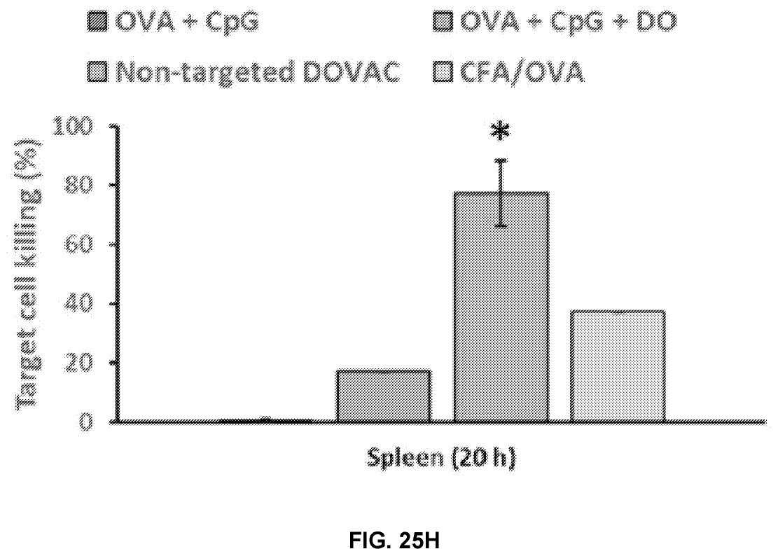

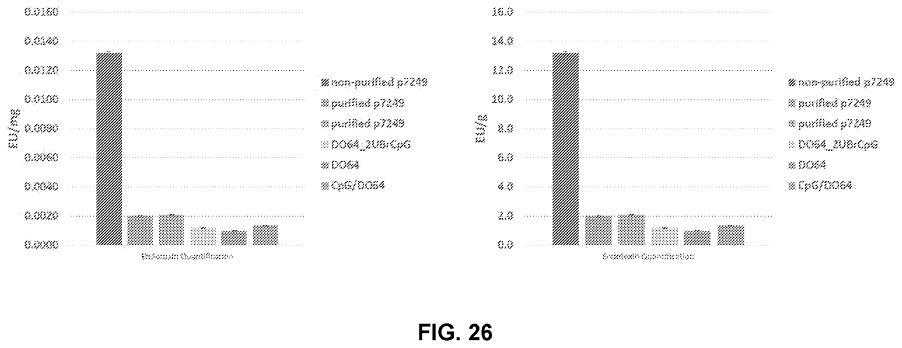

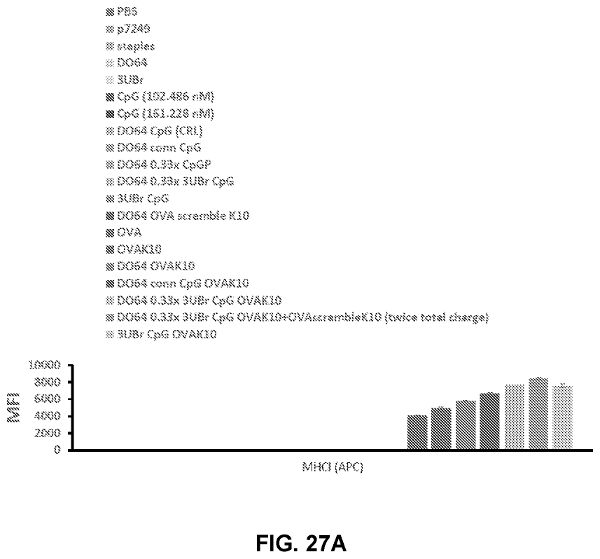

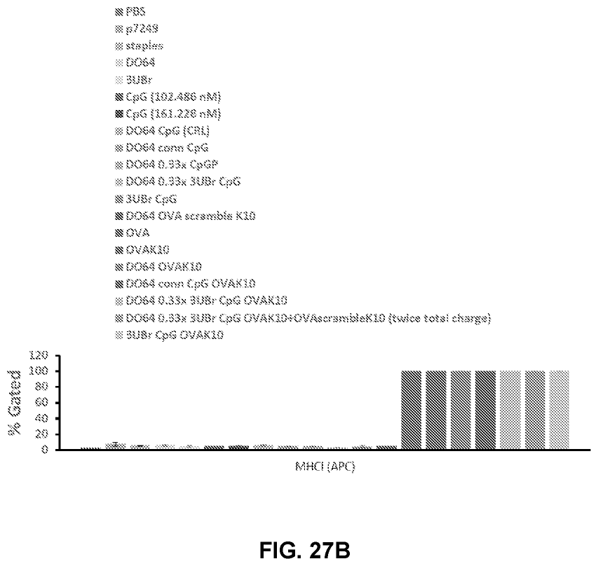

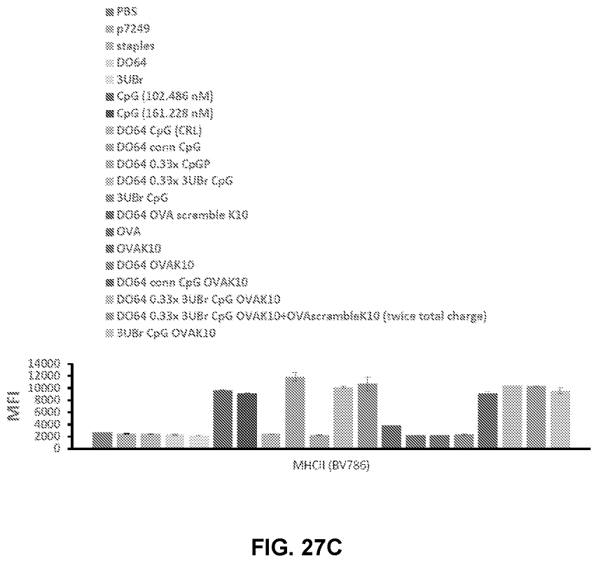

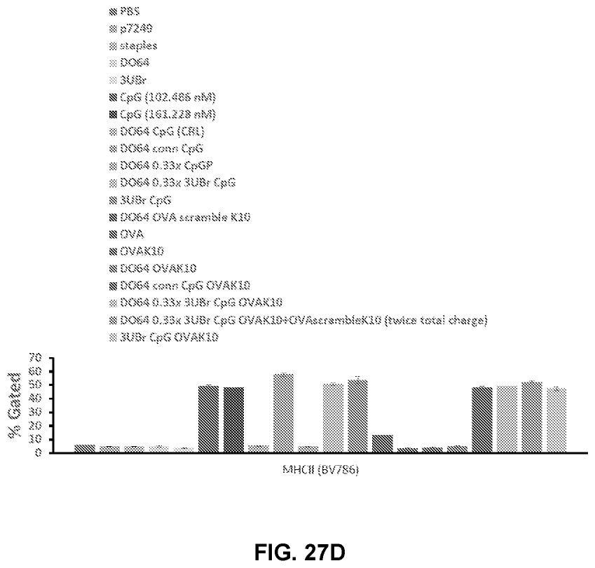

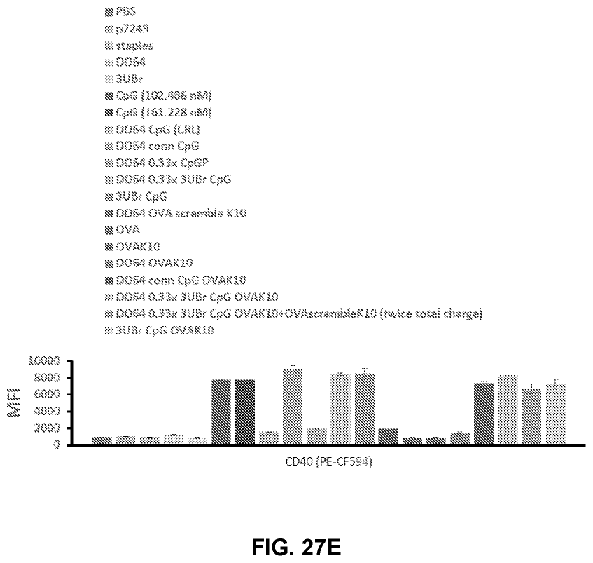

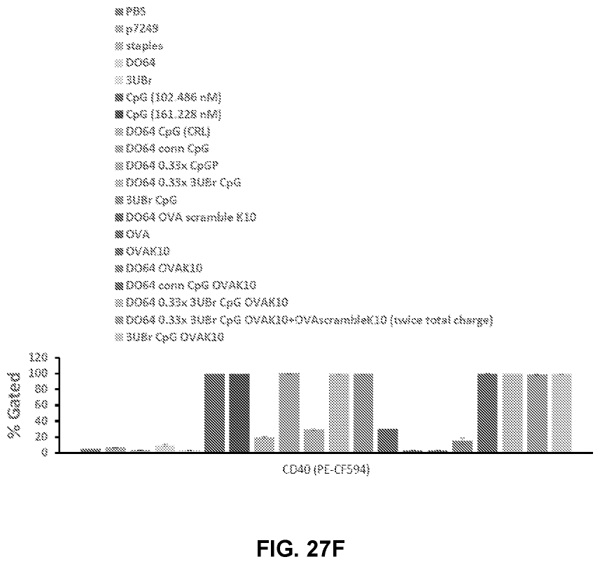

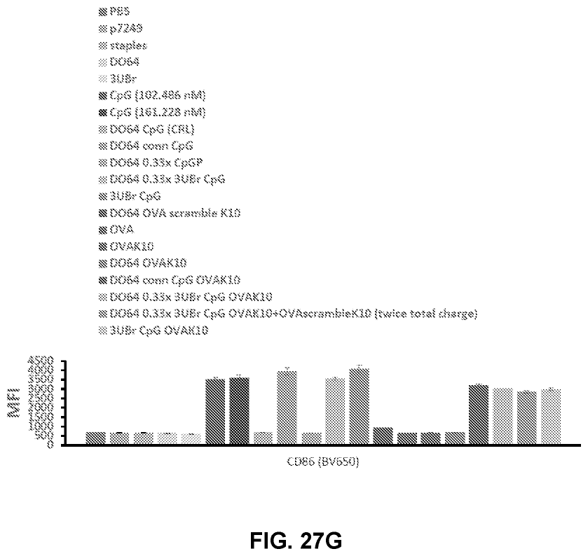

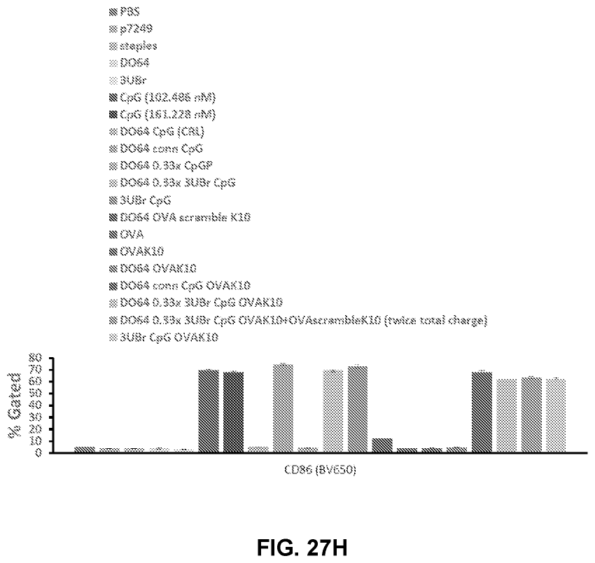

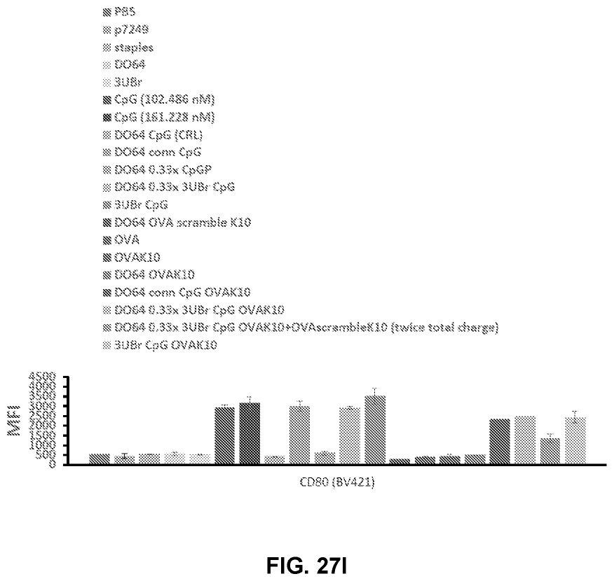

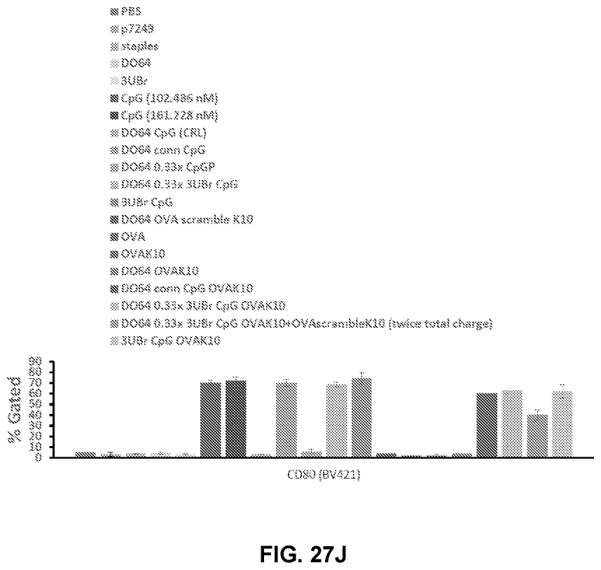

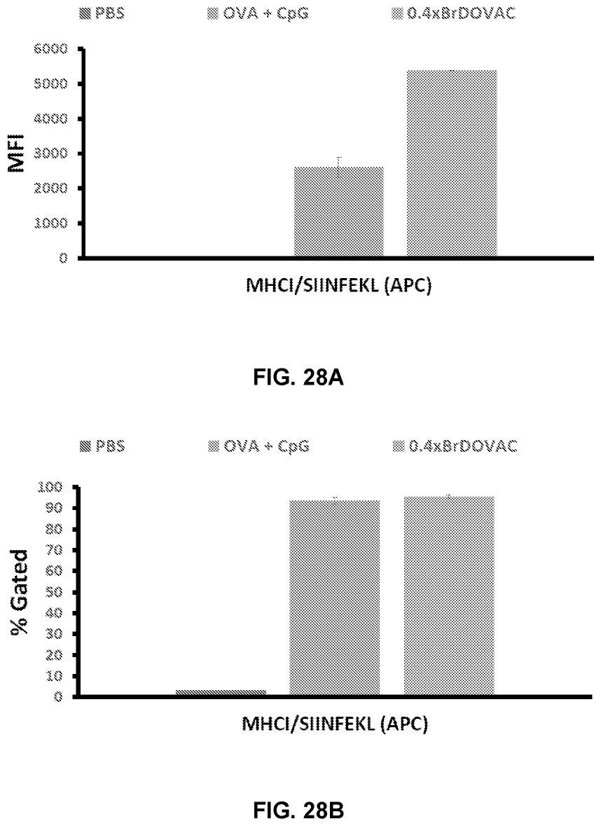

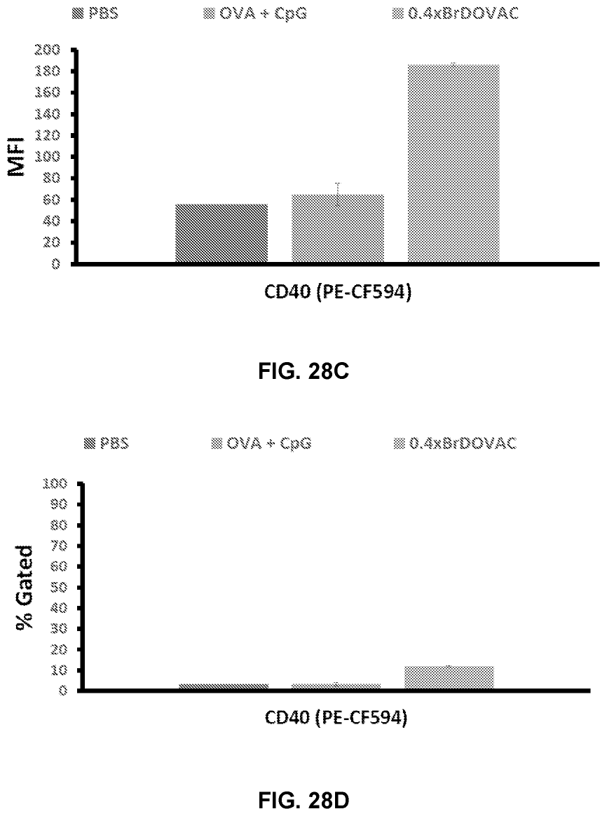

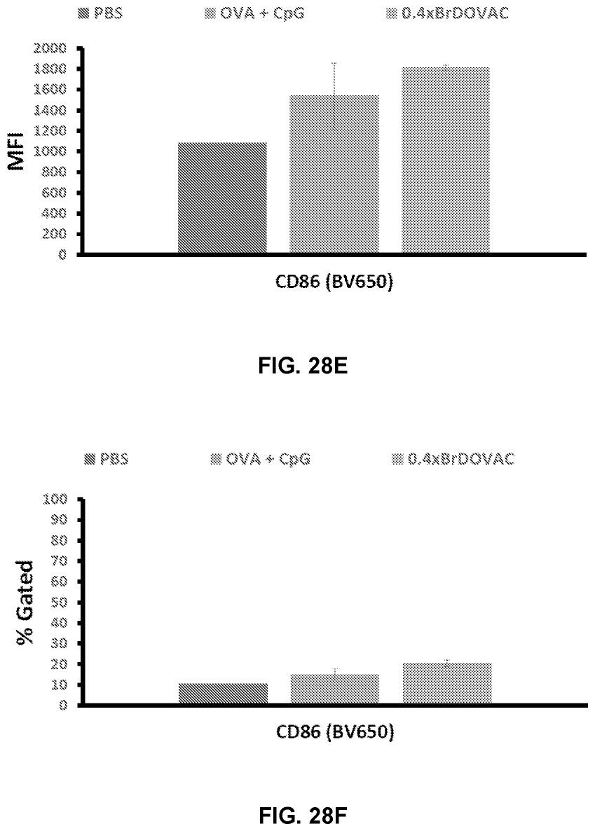

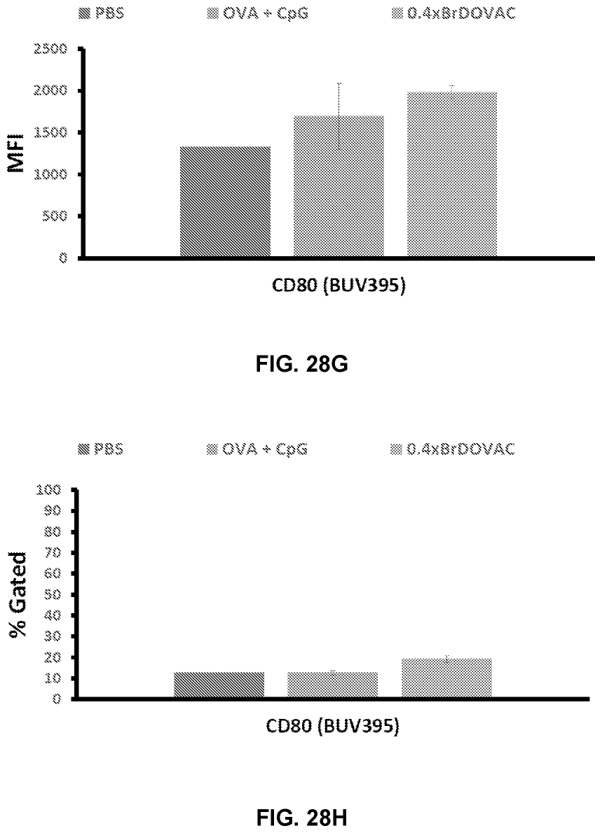

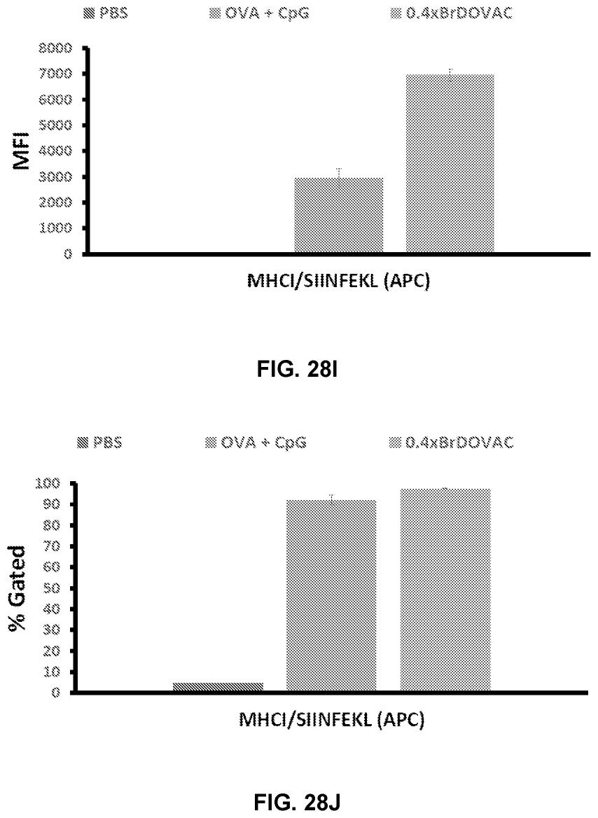

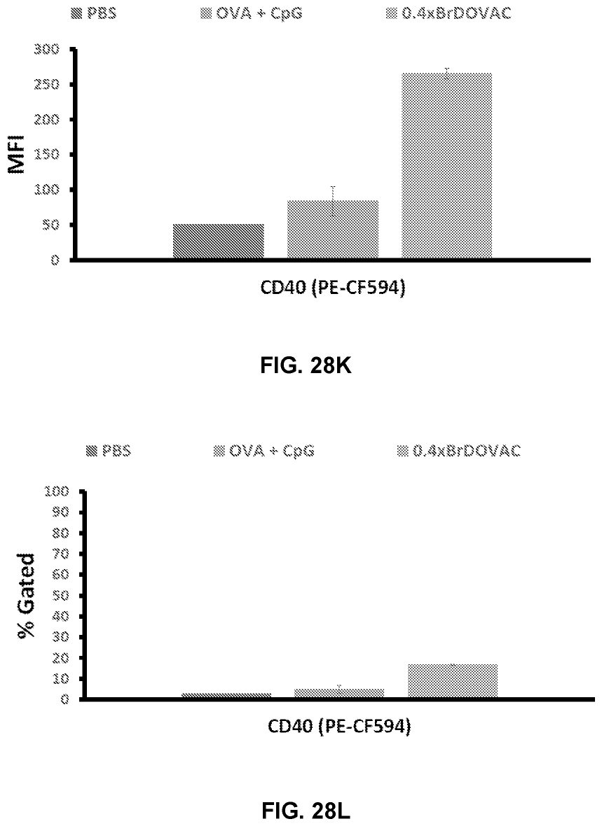

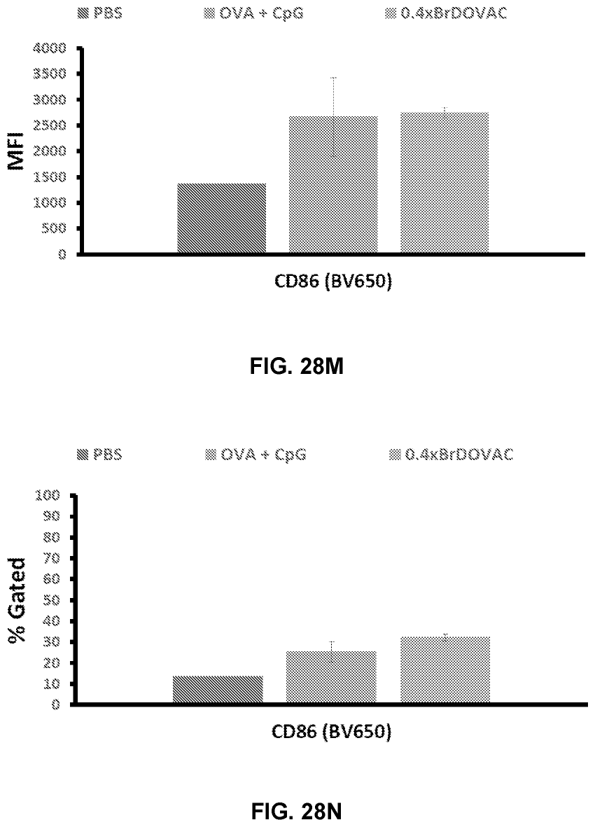

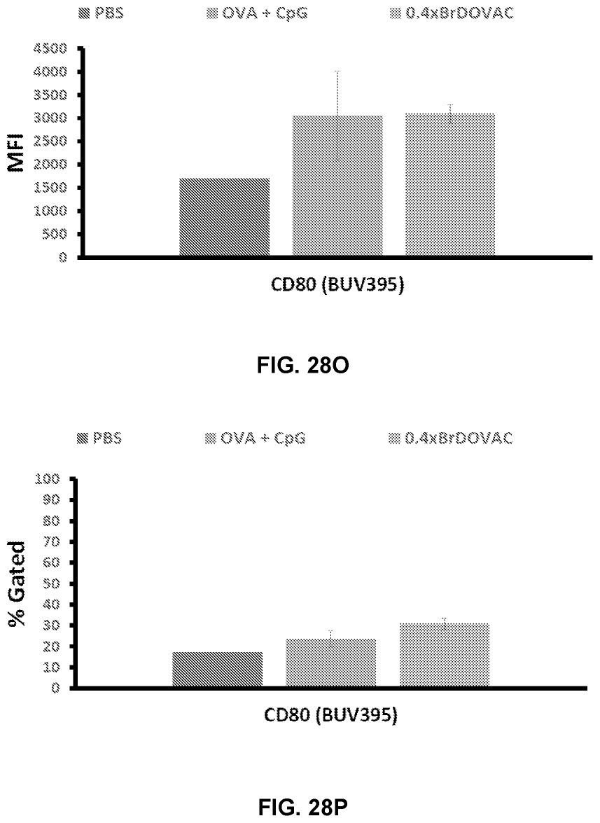

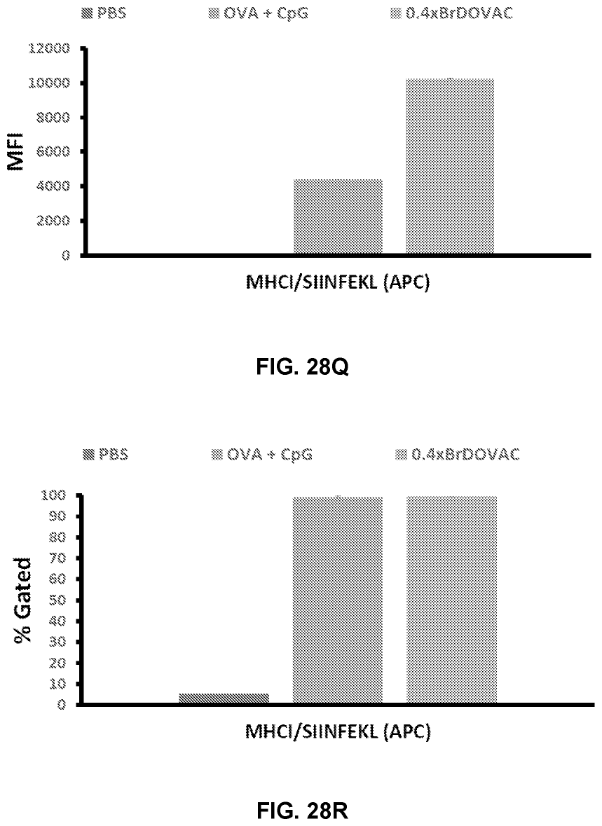

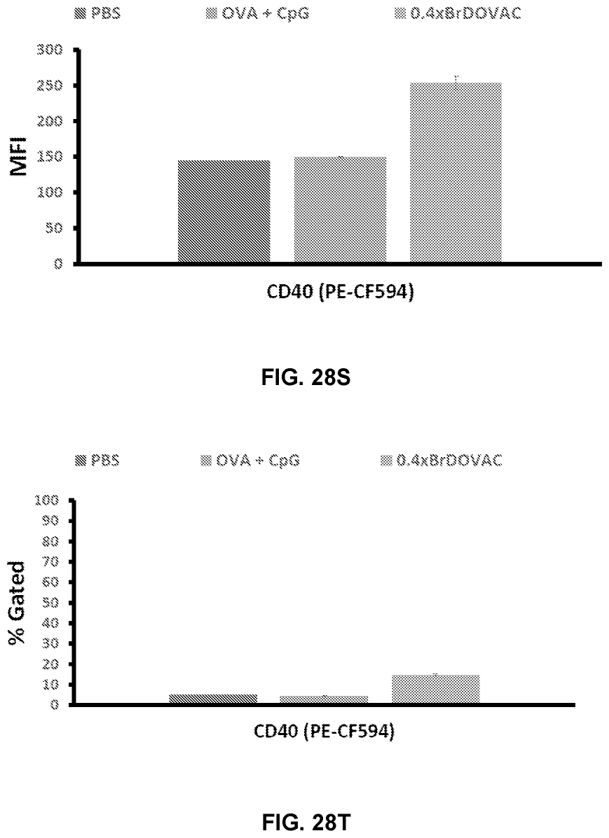

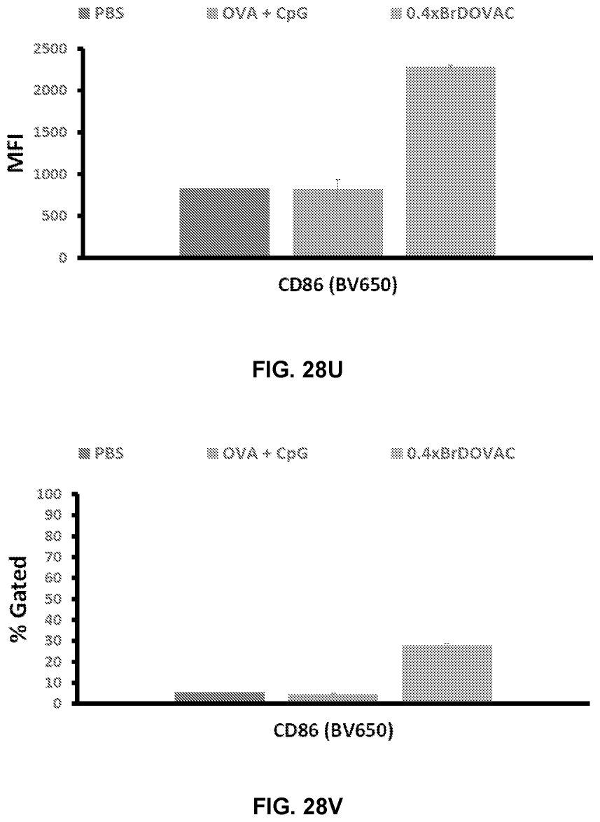

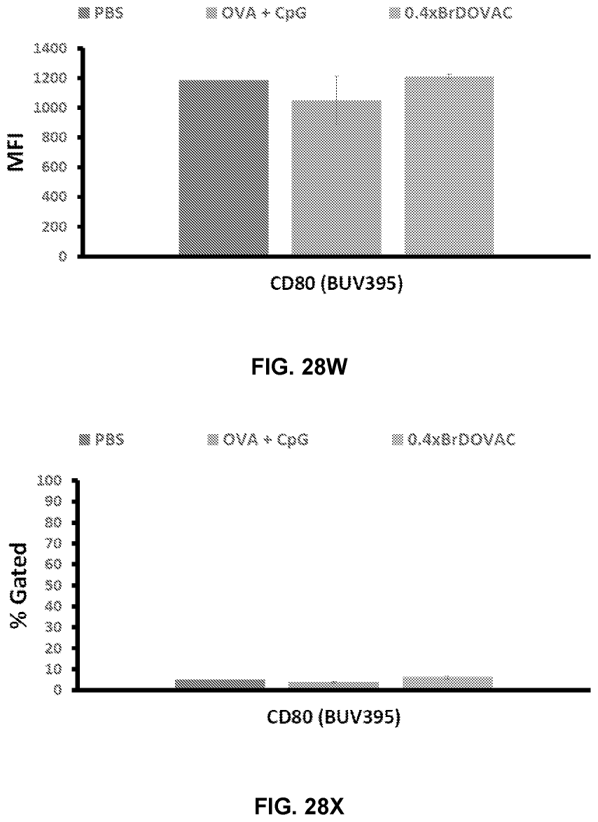

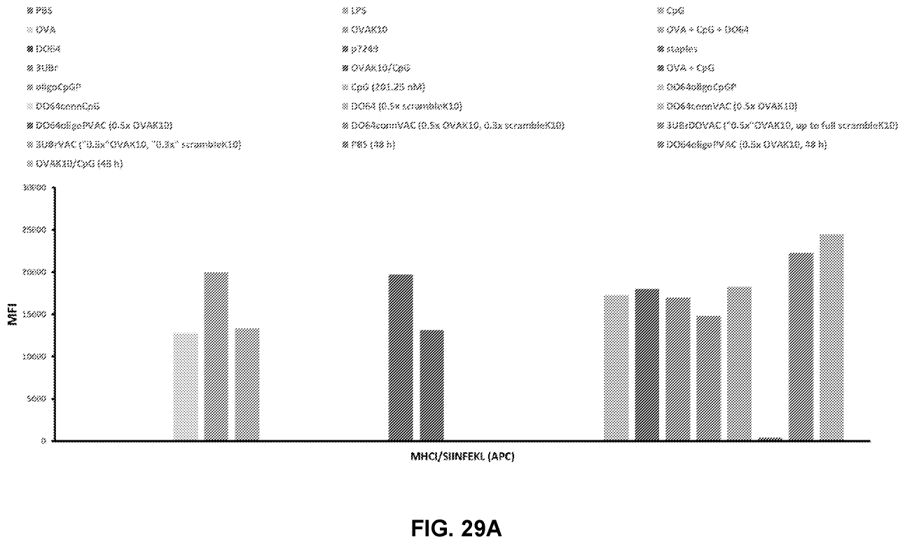

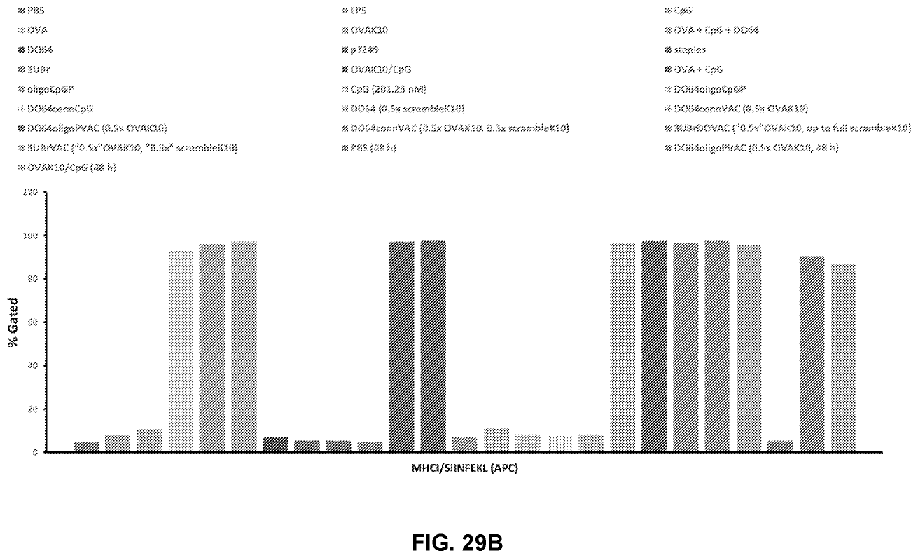

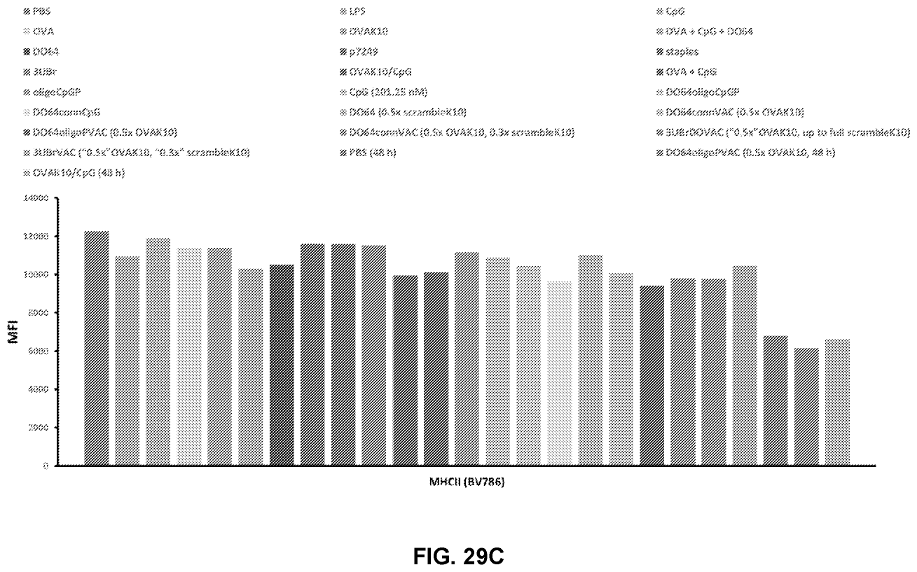

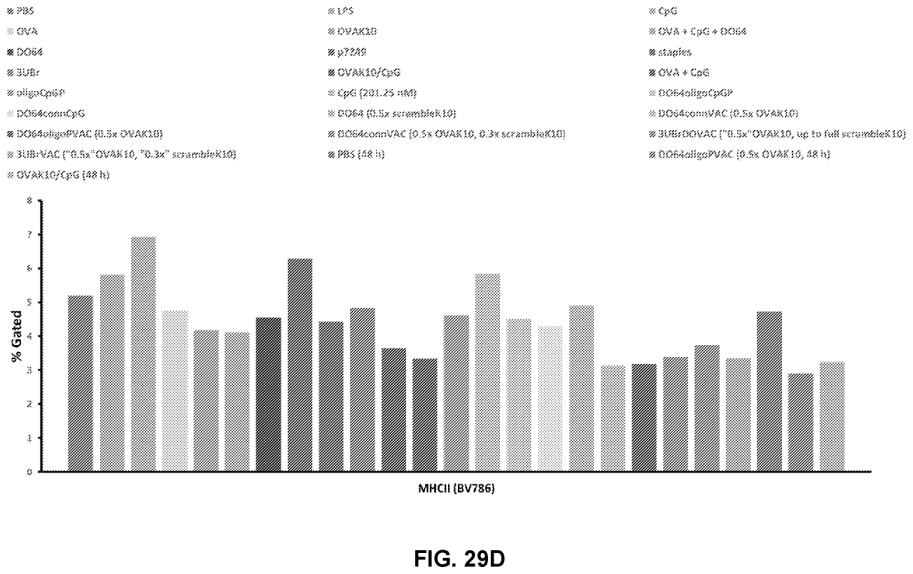

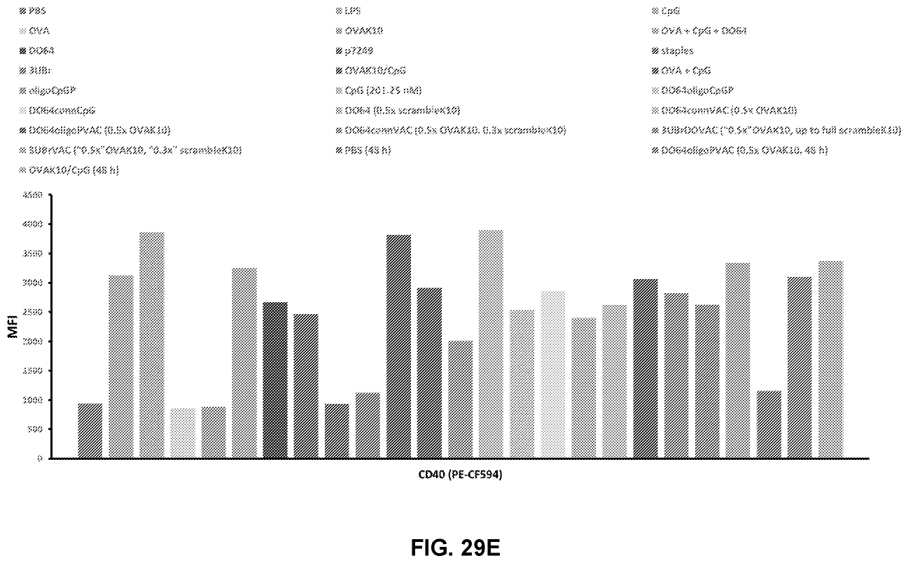

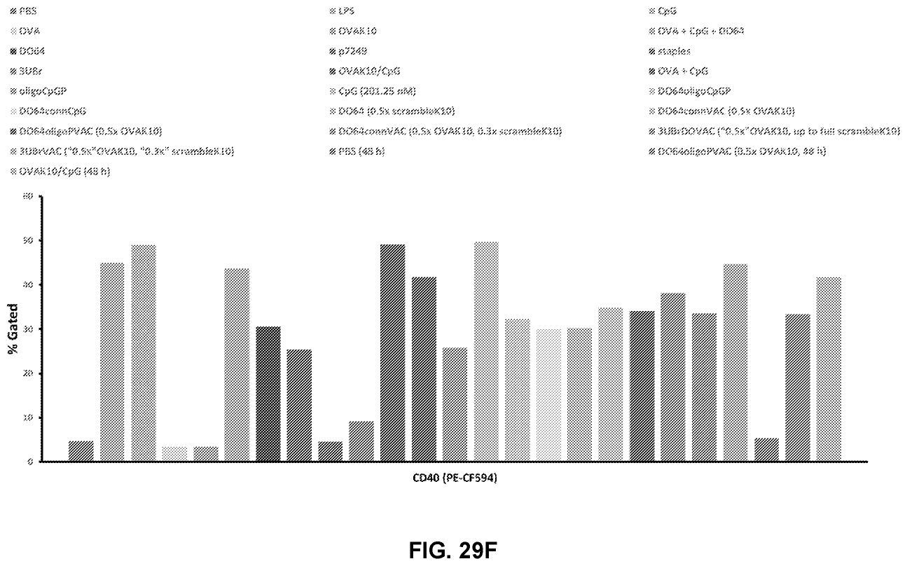

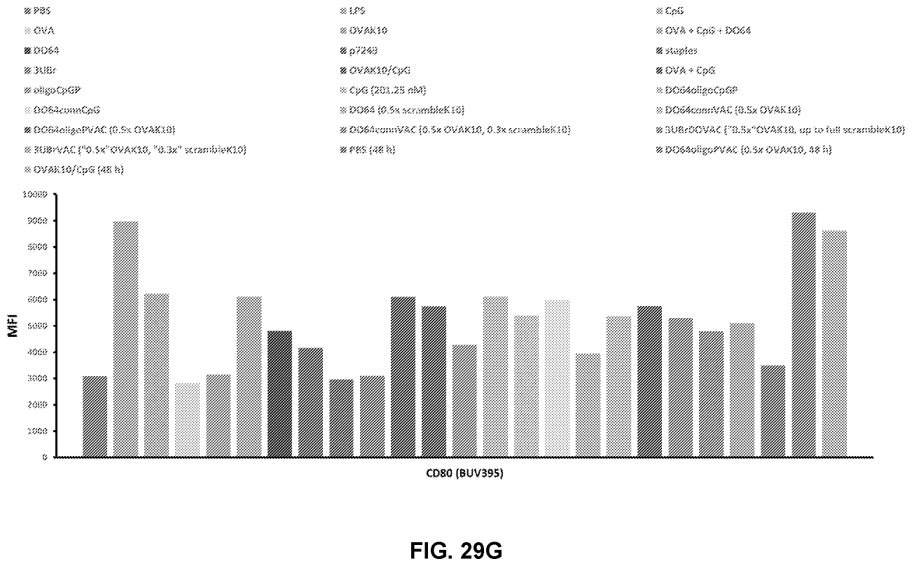

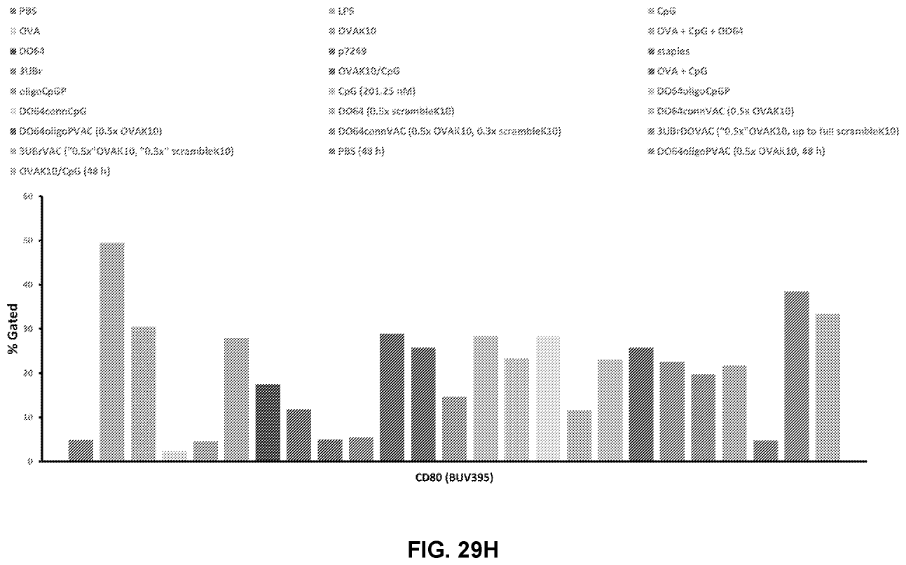

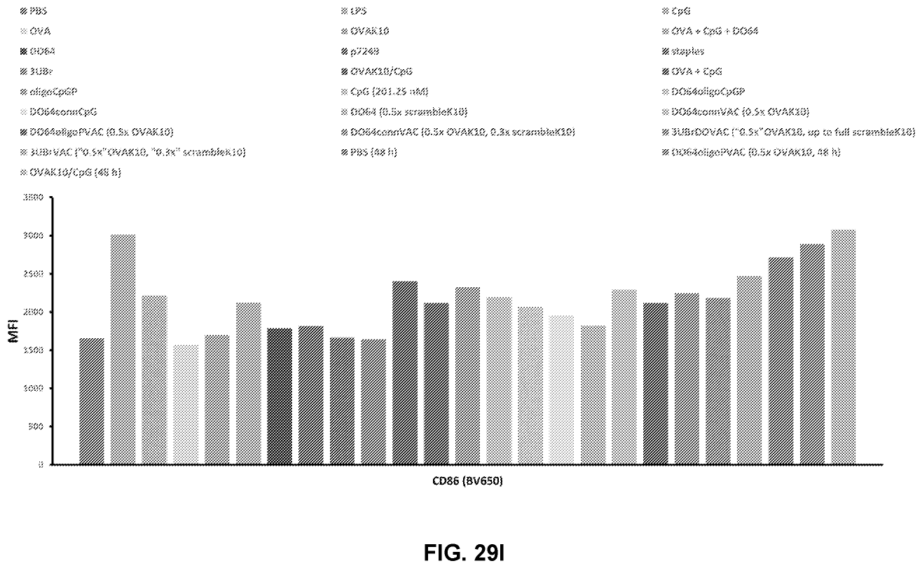

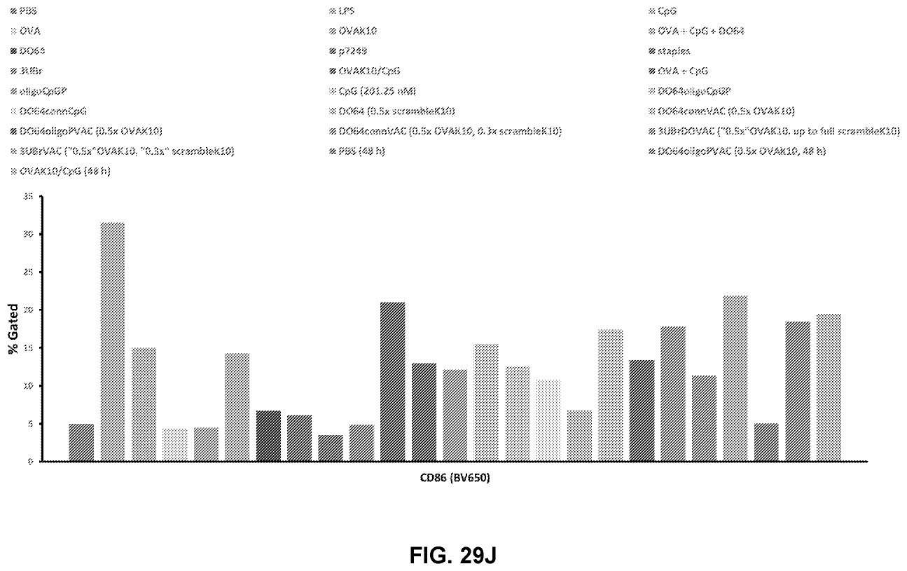

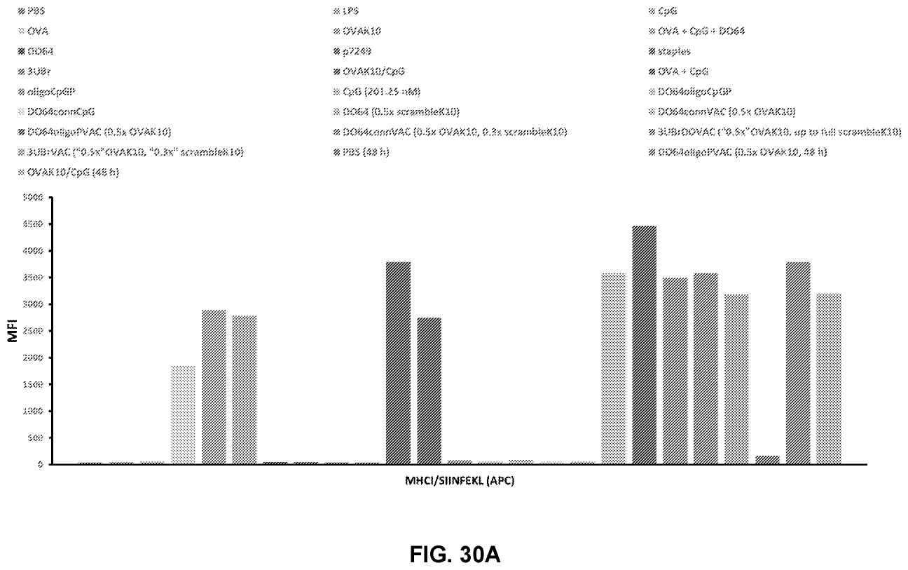

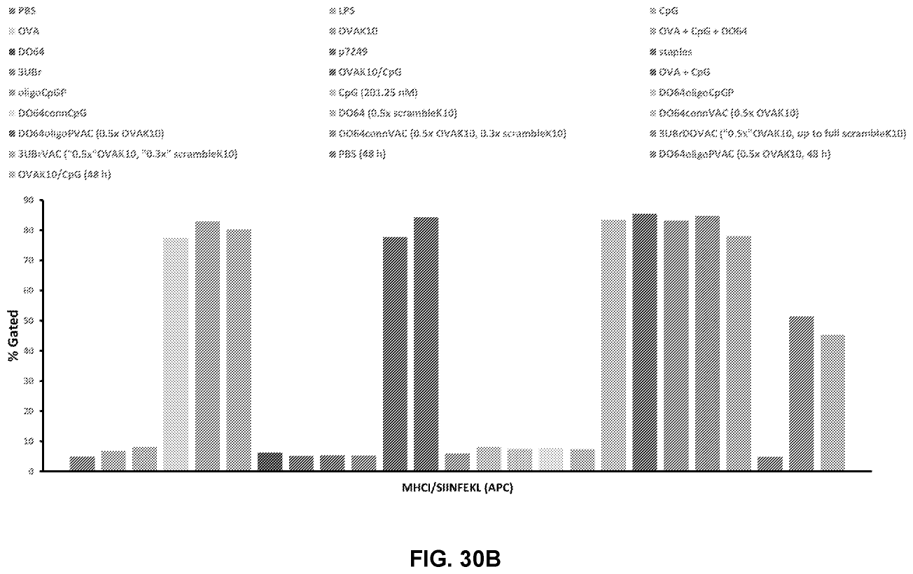

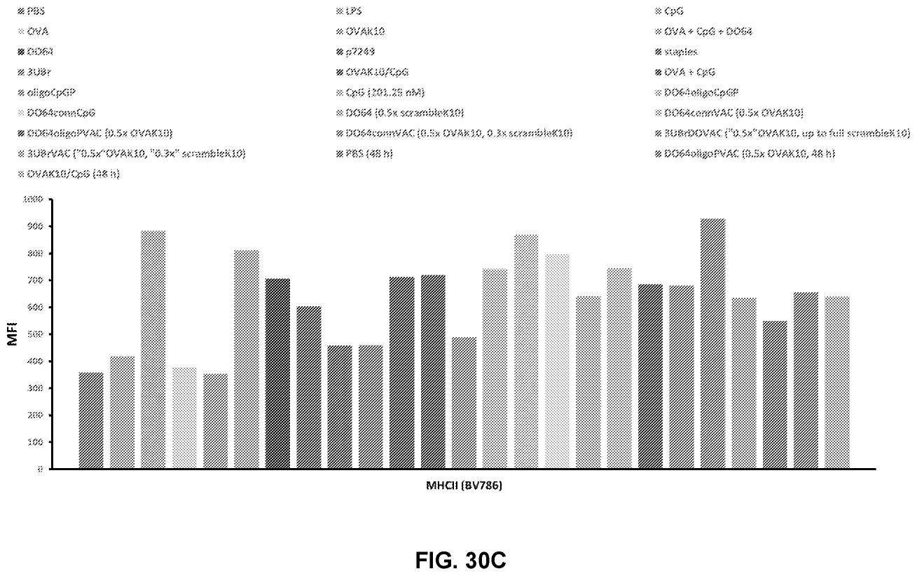

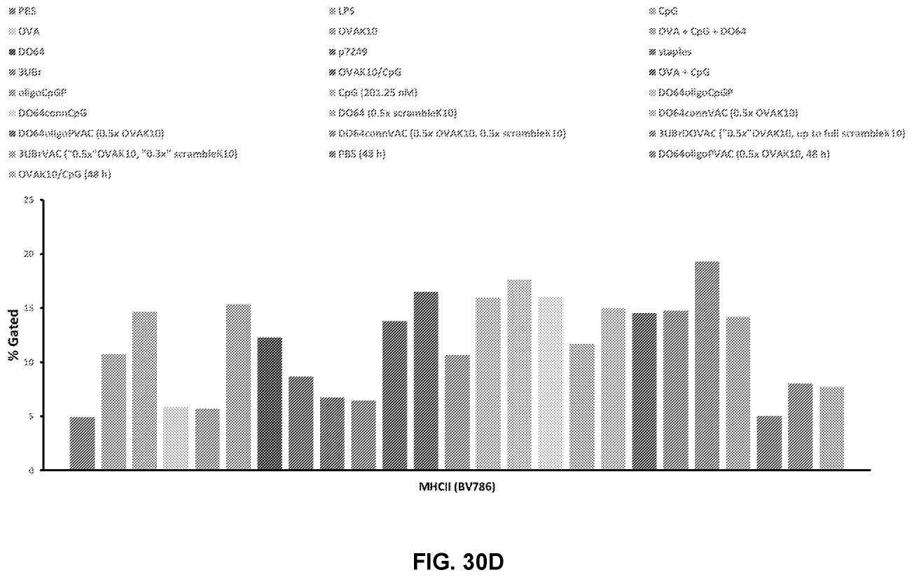

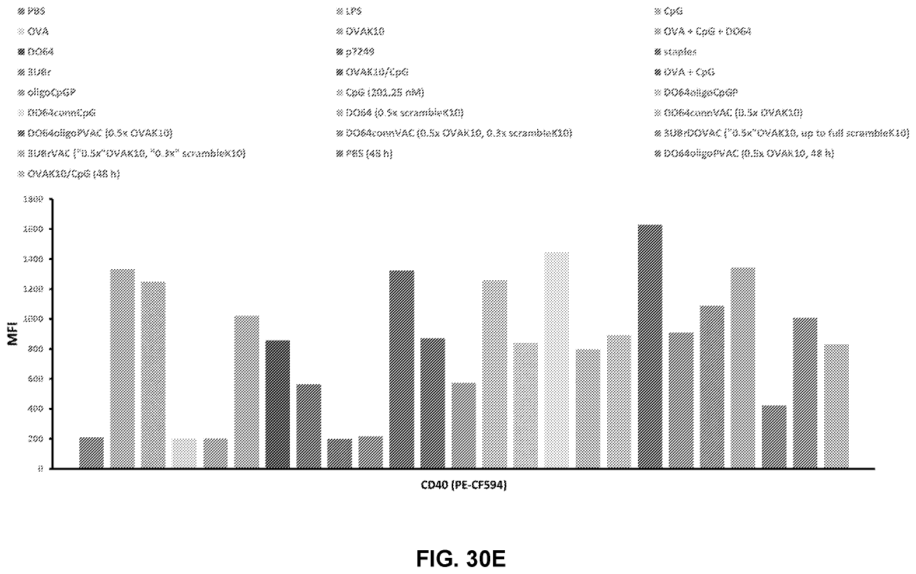

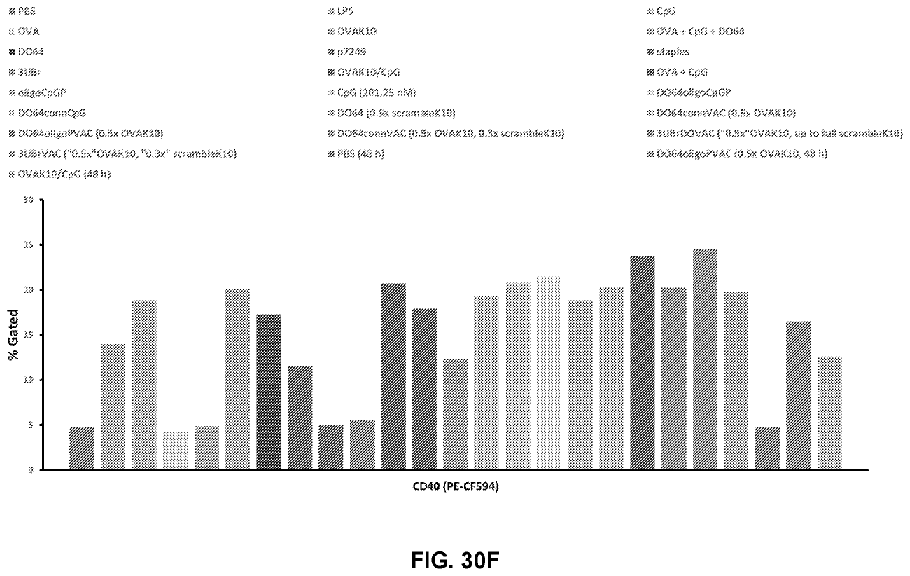

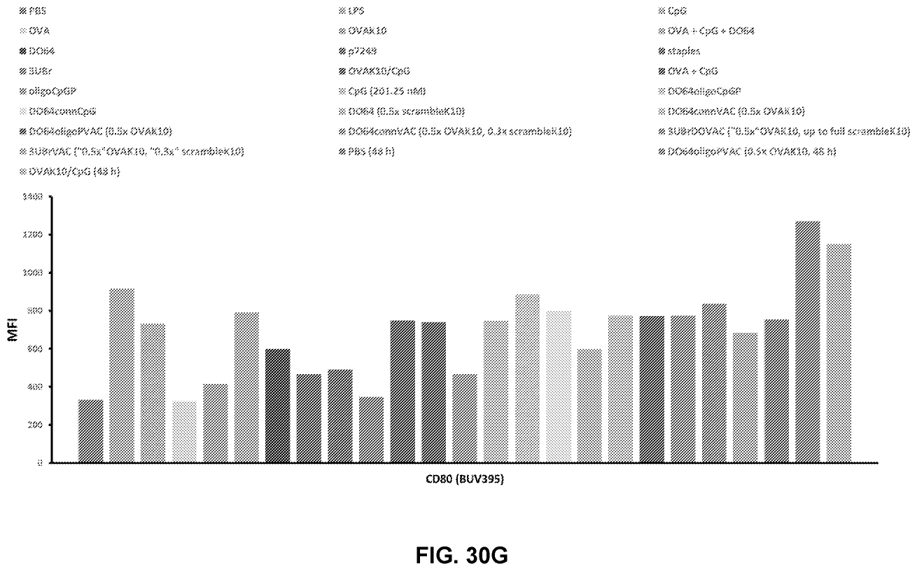

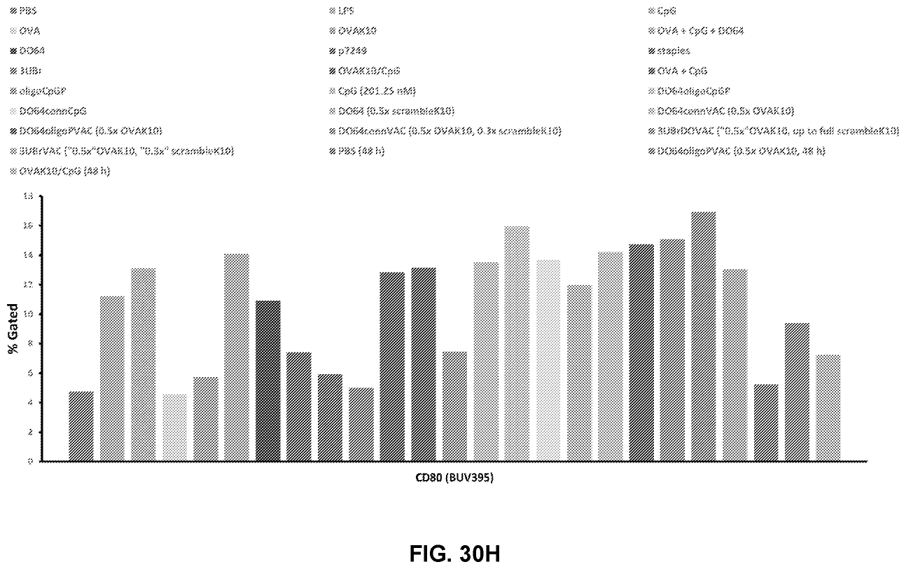

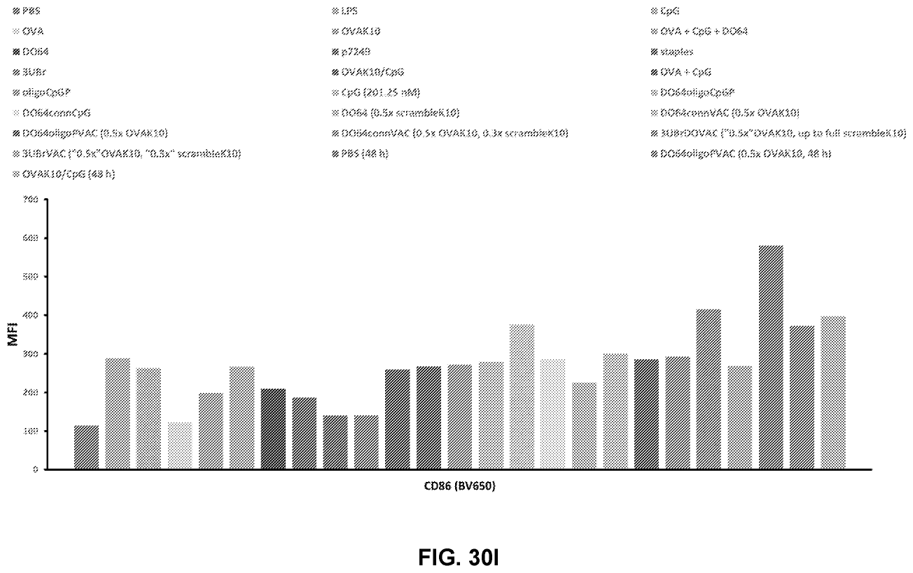

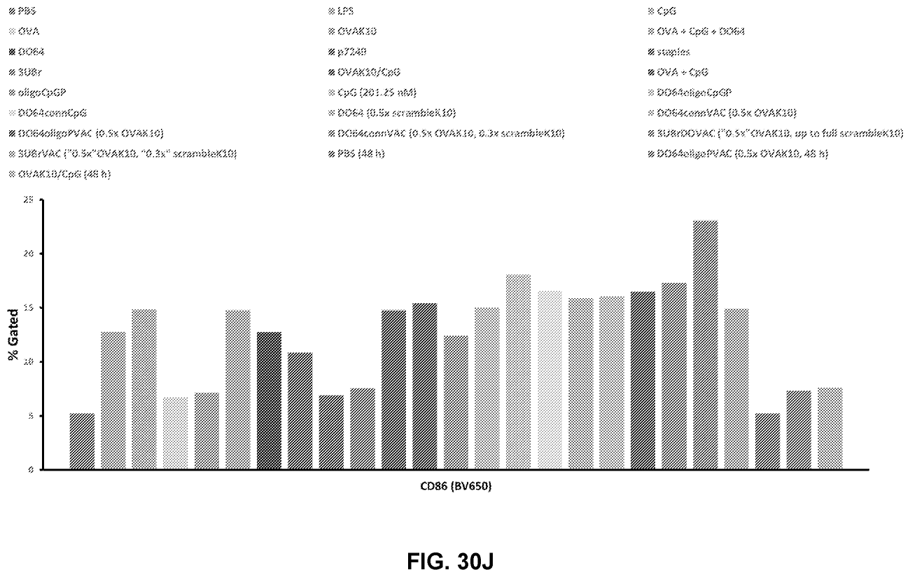

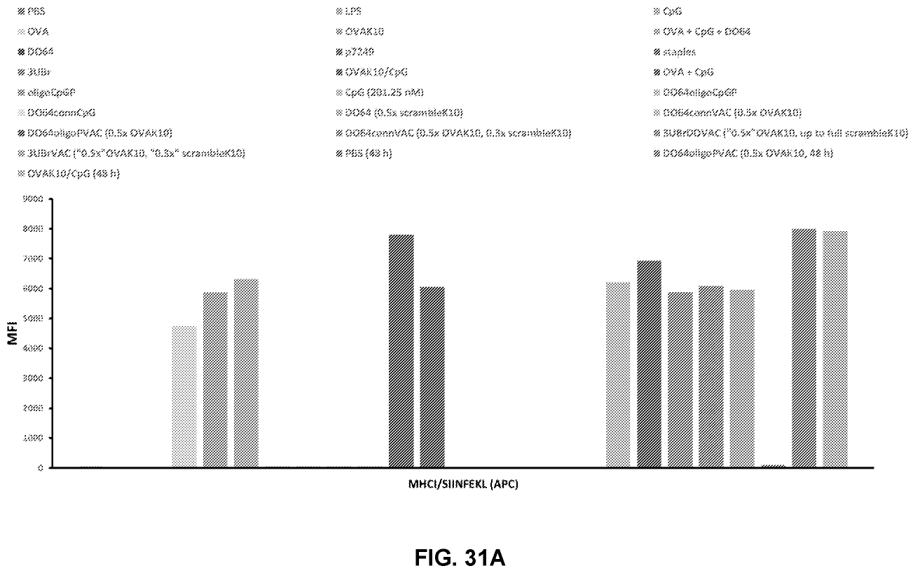

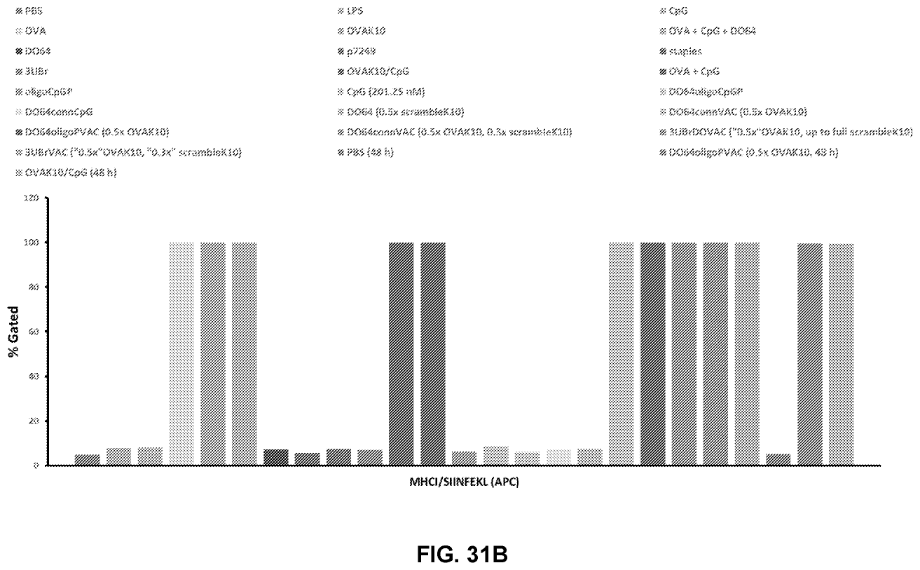

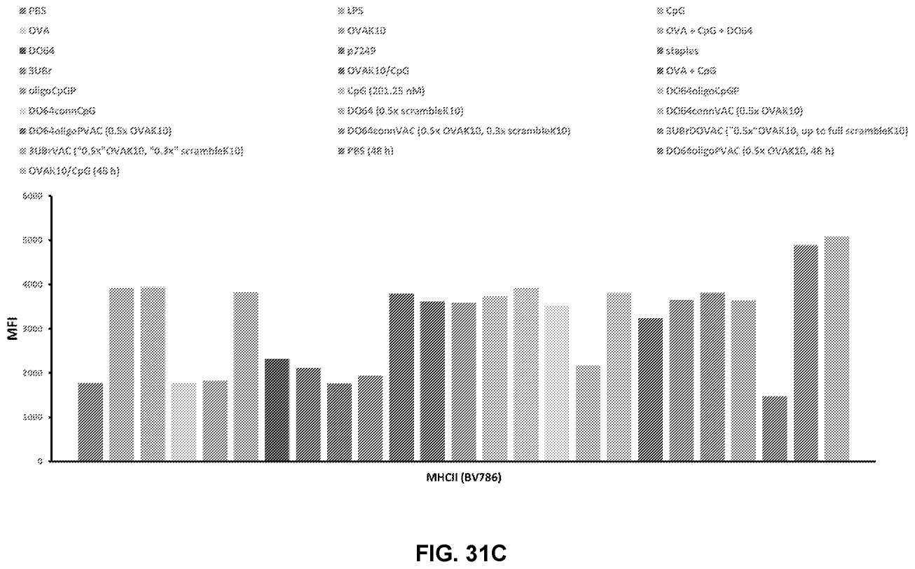

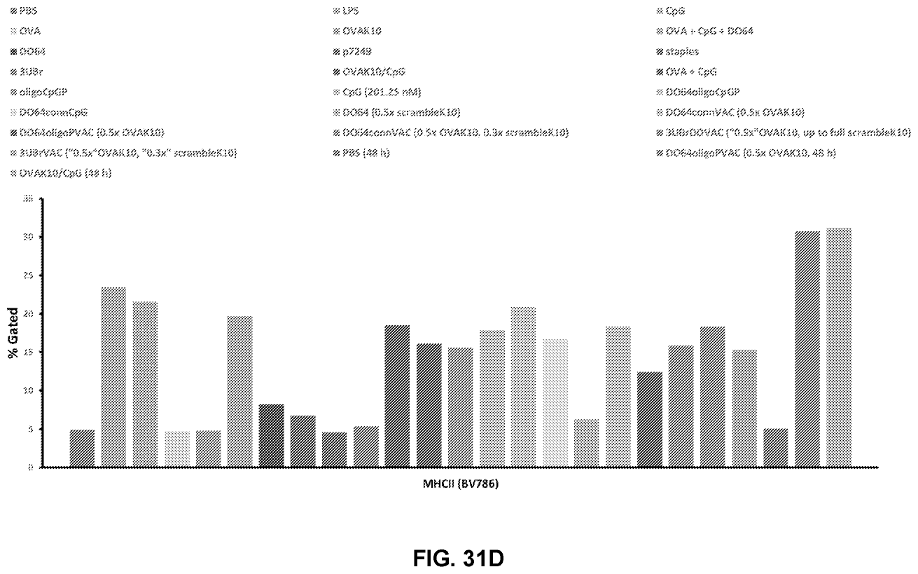

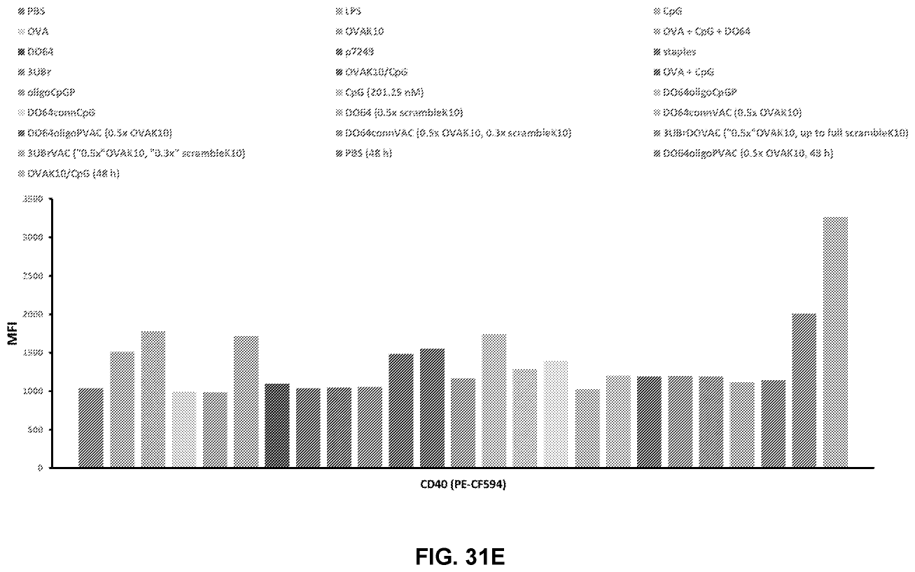

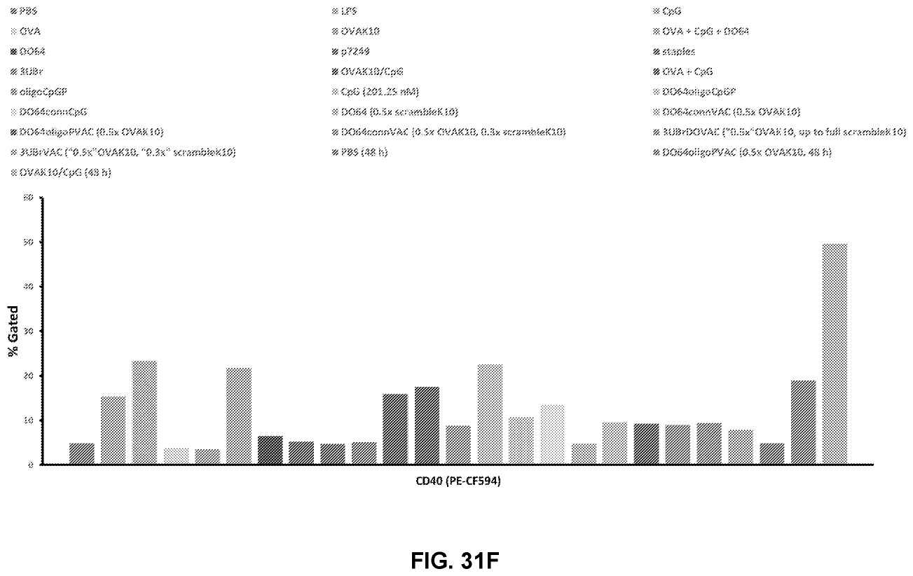

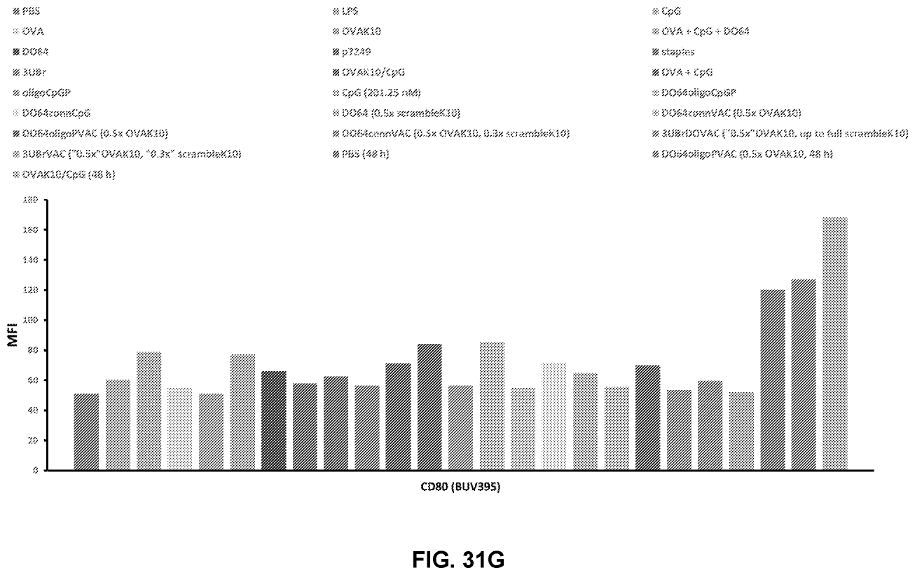

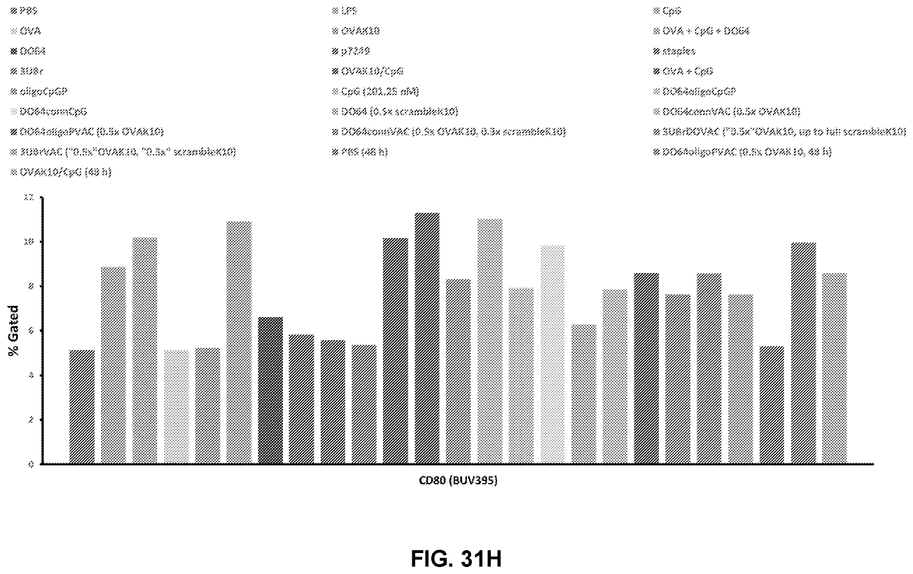

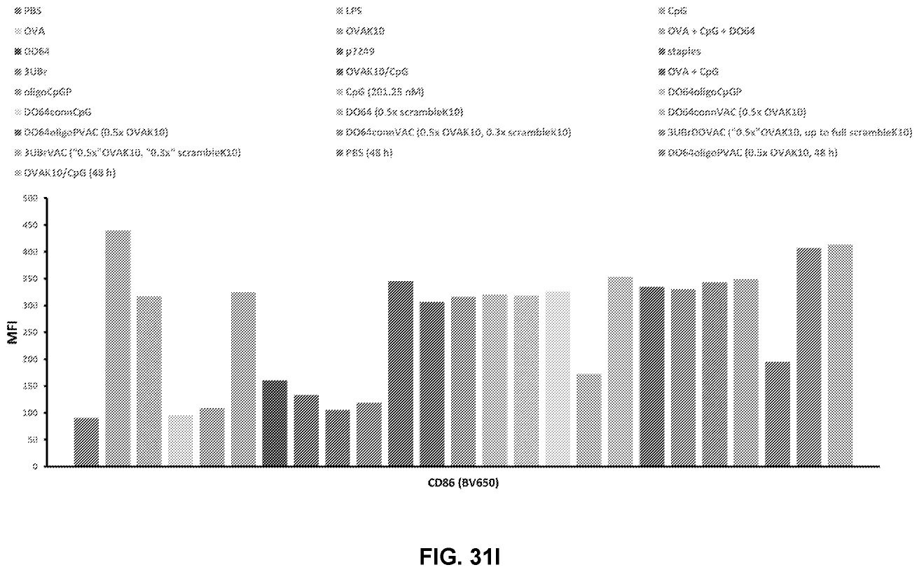

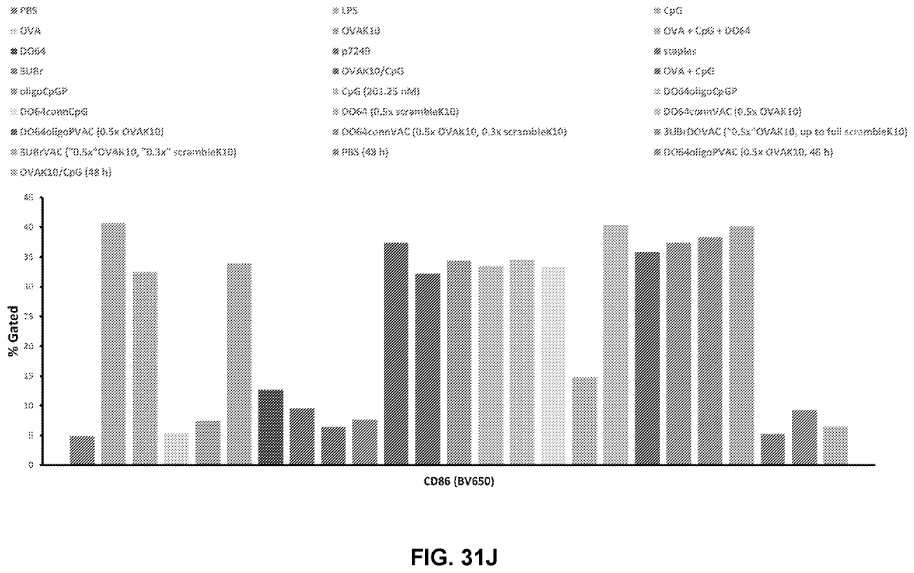

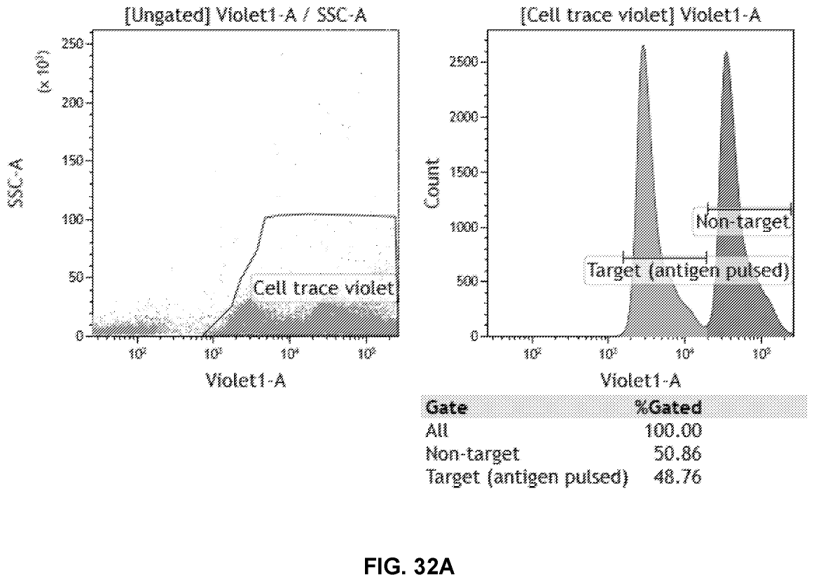

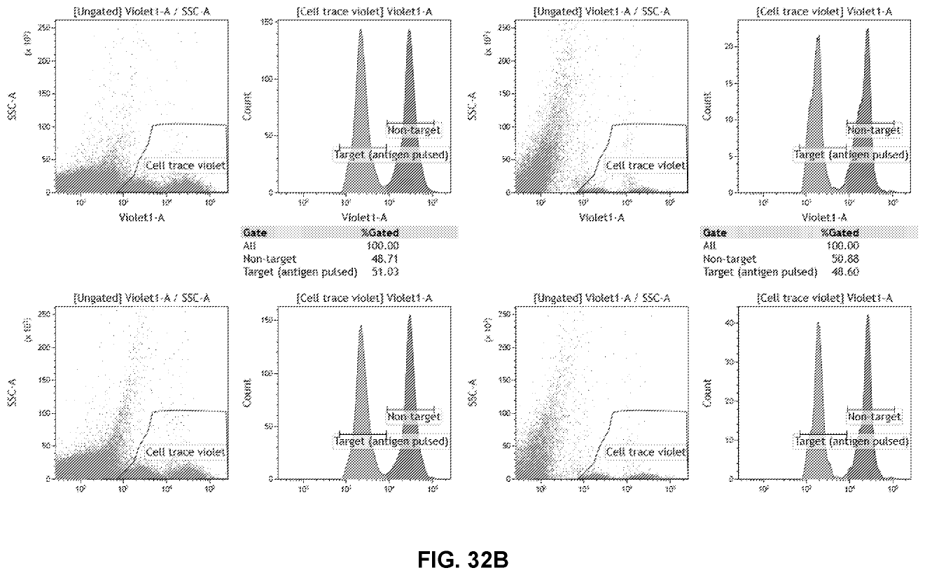

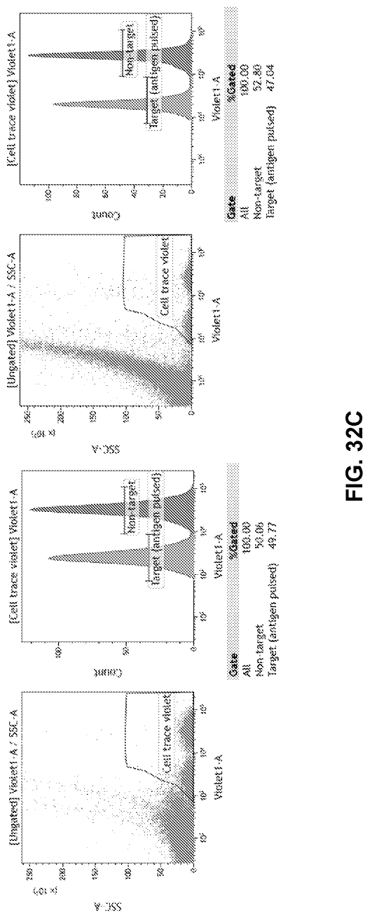

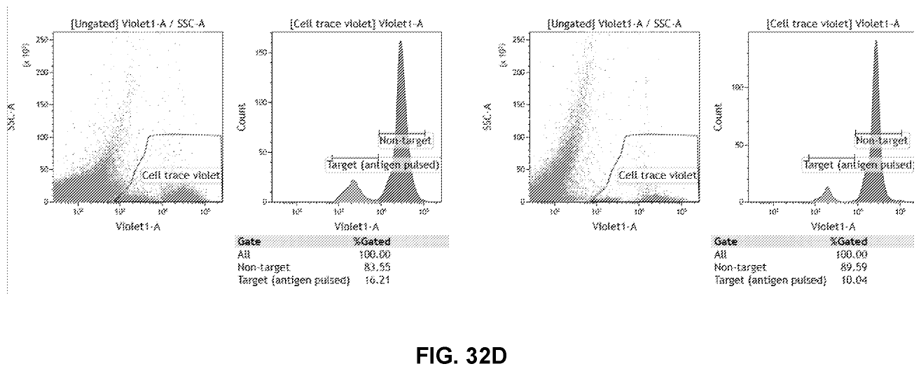

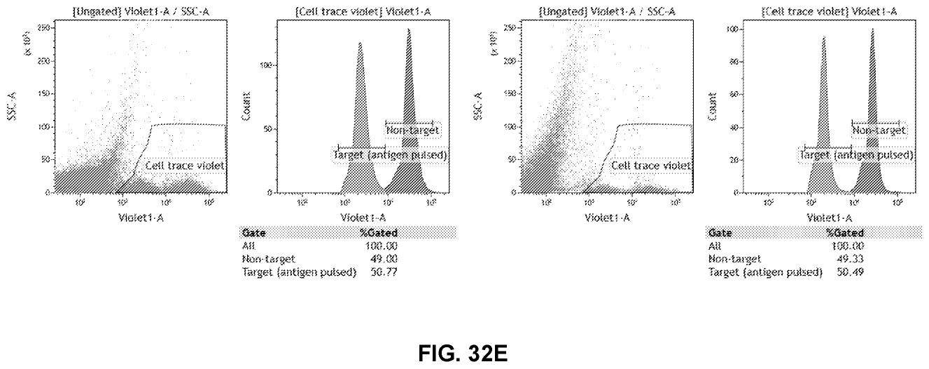

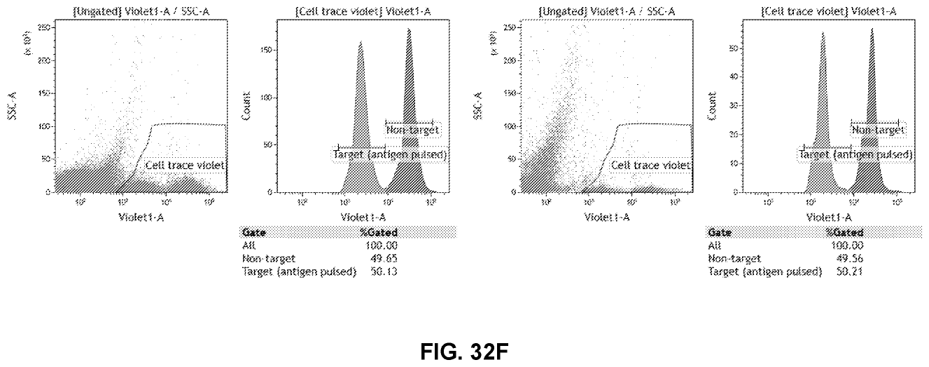

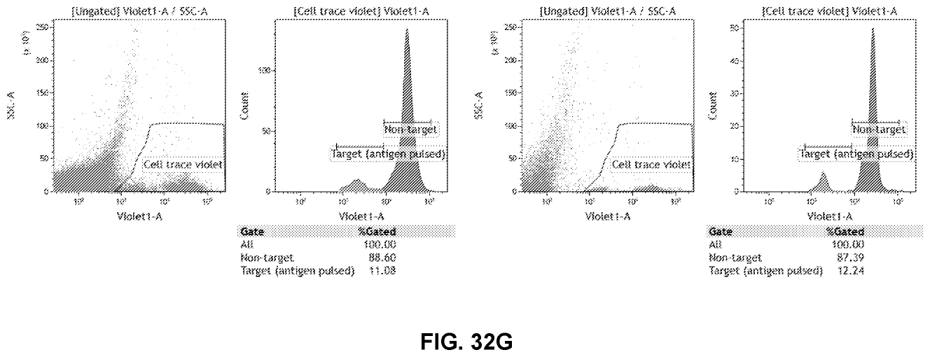

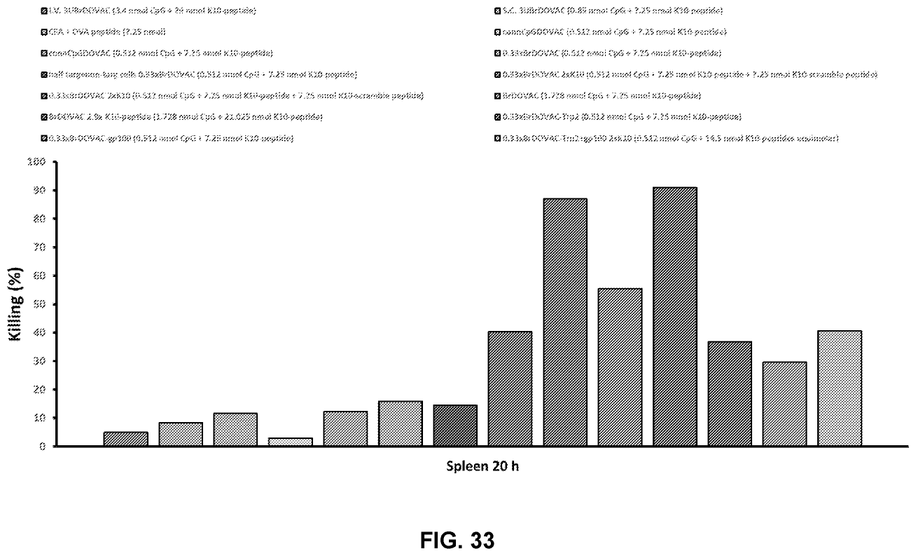

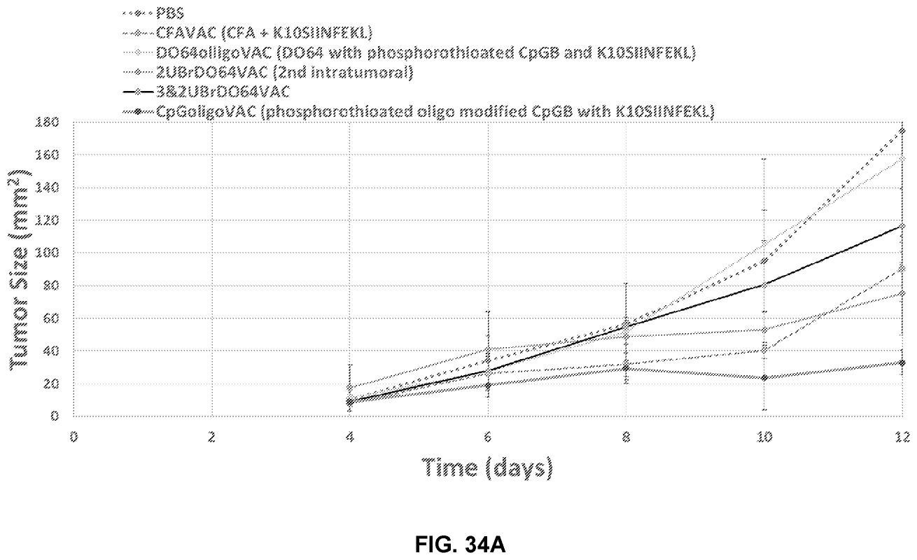

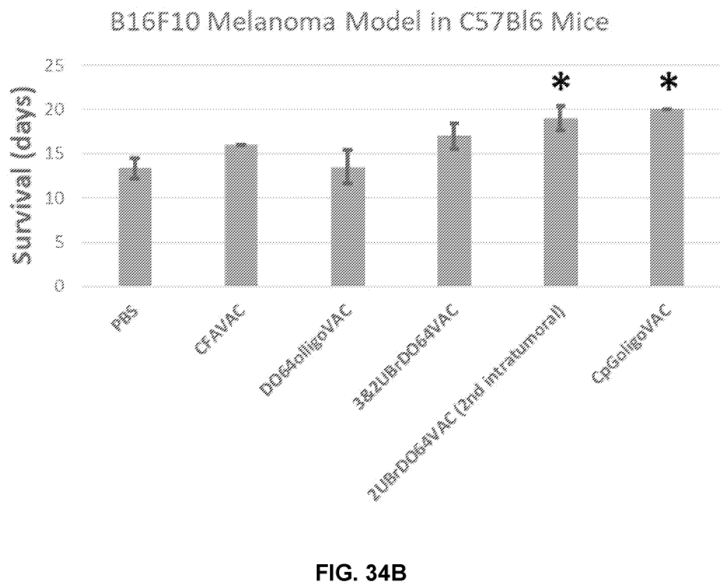

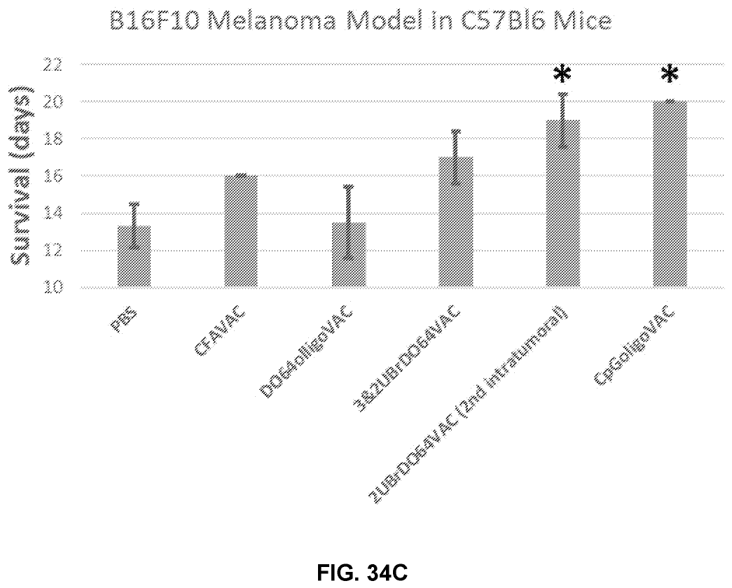

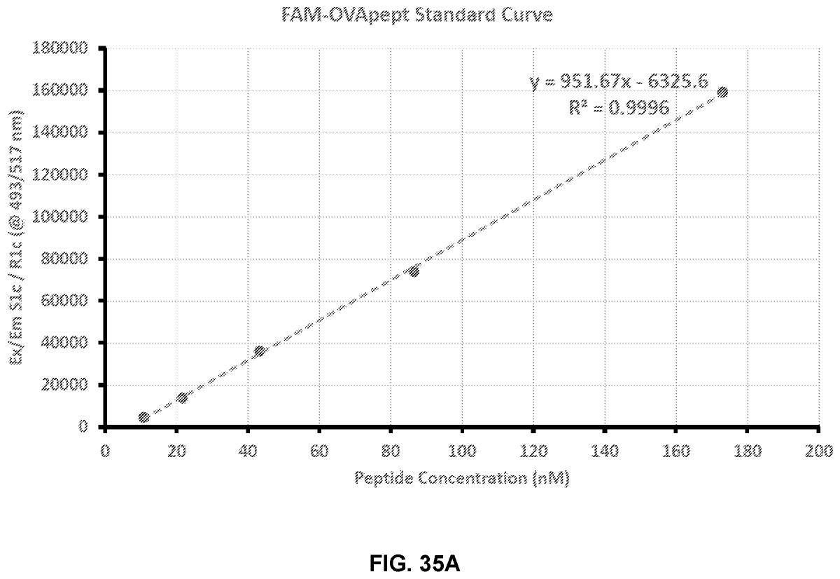

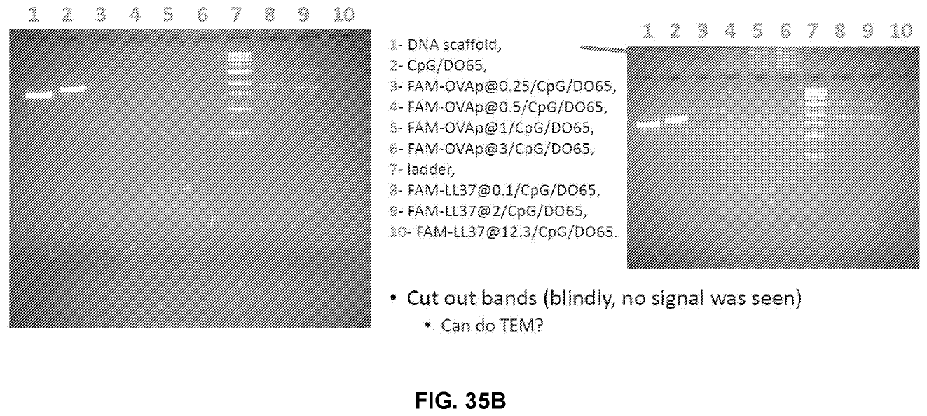

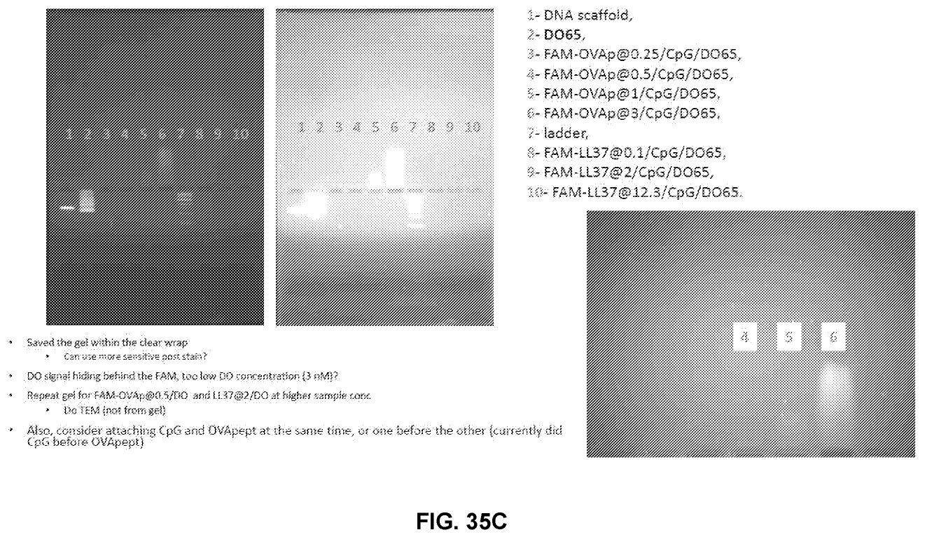

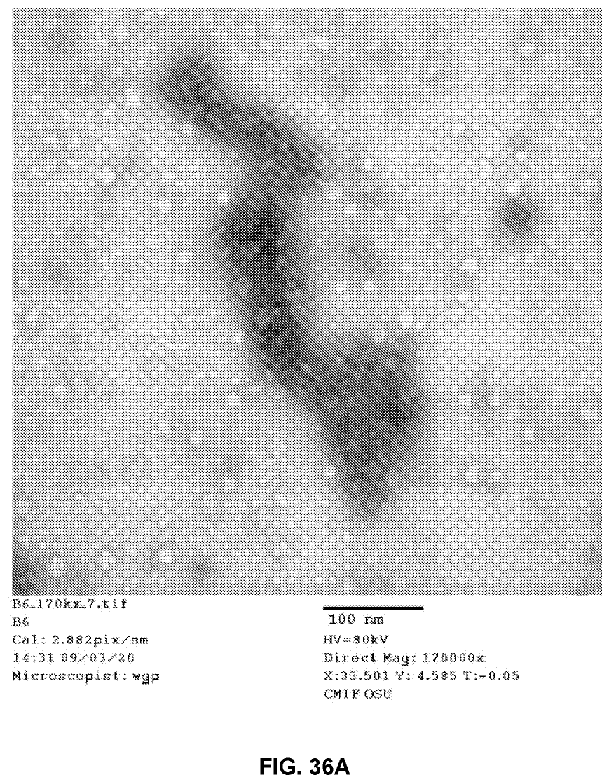

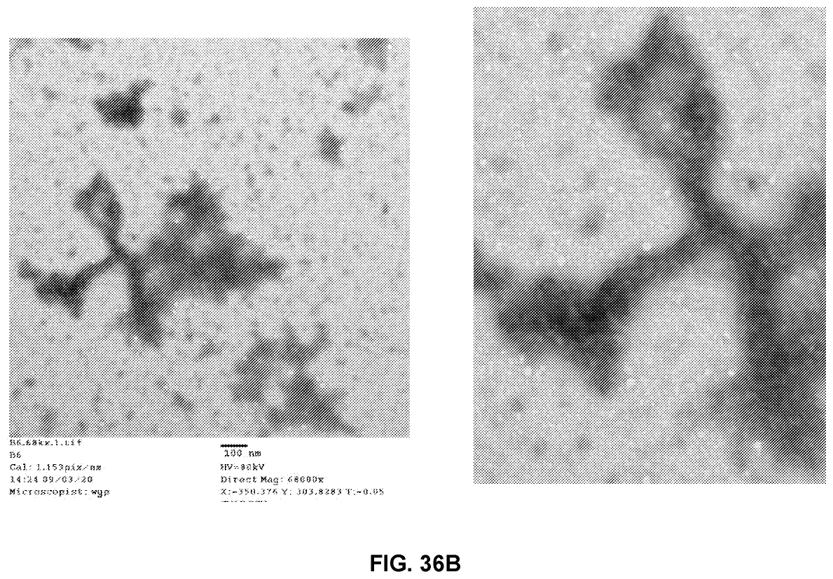

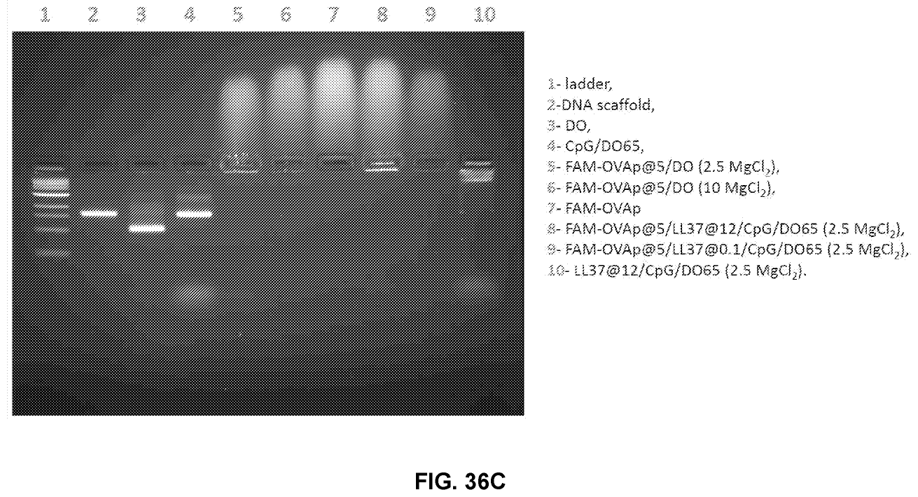

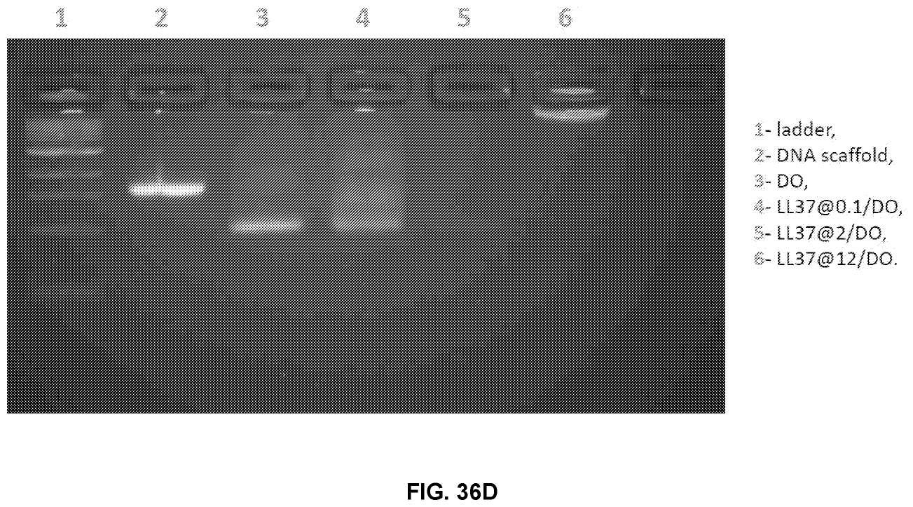

To evaluate effective DO construction and vaccine molecular functionalization, agarose gel electrophoresis showed effective construction and colocalization of DOVAC (V=DOVAC) components and stability in 95% non-heated serum at 37° C. for 0 h-24 h (FIG. 25A). TEM image showed precise and uniform DOVAC geometry, which confirmed effective construction (FIG. 25B). TEM image showed Ab attached to DOVAC utilizing a medium number of attachment sites suggesting effective Ab functionalization (FIG. 25C). Flow cytometry of antigen presentation and activation markers in DCs in vitro revealed DOVAC mediated elevation suggesting effective APC activation (FIG. 25D). DOVAC showed enhanced delivery to DCs via APC targeting ligands in vitro (FIG. 25E). Flow cytometry of OVA-specific T cell (from OT-1 mice) revealed expansion 3 days after their adoptive transfer in WT mice and mice S.C. injection of either buffer, SIINFEKL (SEQ ID NO:2) (OVA)+CpG mixture, or DOVAC (FIG. 25F). T cell activation markers shown in (FIG. 24G). We then assessed CD8+ T cell mediated cell killing with flow cytometry in vivo 7 days post S.C. injection of indicated conditions. Data shown as mean (% gated)+SEM and n3.

Collectively, these findings demonstrate 1) the DOVAC vehicle alone is safe and mildly immunogenic; 2) effective construction and functionalization of a DOVAC with adjuvant and antigen; 3) DOVAC effective delivery to APCs; 4) DOVAC effective APC cell activation and antigen-specific T cell expansion; and 4) DOVAC is functional in vivo to elicit targeted CD8+ T cell mediated killing.

### Example 7

Nanoparticle-(NP)-based vaccine technologies have shown promising potential in cancer treatment due to their versatile and unique properties that are relevant in antigen delivery and presentation. Liposomes and various other NPs made of polymers (e.g., PLGA, PEG, dextran), inorganic materials (e.g., silica, carbon, gold), and other materials, have advantageous and tunable properties related to their size, shape, and surface, which can be modified with functional molecules (Benne, N, et al. Journal of Controlled Release 2016 234:124-134; Albanese, A, et al. Annual Review of Biomedical Engineering 2012 14:1-16; Hoshyar, N, et al. Nanomedicine (Lond) 2016 11:673-692; Bastings, M M C, et al. Nano Letters 2018 18:3557-3564; Khisamutdinov, E F, et al. Nucleic Acids Research 2014 42:9996-10004; Lundqvist, M, et al. PNAS 2008 105:14265-14270; Schüller, V J, et al. ACS Nano 2011 5:9696-9702; Halley, P D, et al. Small 2016 12:308-320; Afonin, K A, et al. Nature Nanotechnology 2013 8:296-304; Goldinger, S M, et al. Eur J Immunol 2012 42:3049-3061; Giljohann, D A, et al. Nano Letters 2007 7:3818-3821; Zhang, K, et al. J Am Chem Soc 2012 134:16488-16491). These properties have been shown to modulate NP pharmacokinetics, biodistribution, solubility, and targeting in order to deliver therapeutics/vaccines in a cell-specific manner and improve therapeutic efficacy while reducing side effects. Also, NPs have been used as multifunctional systems for the co-delivery of immunostimulatory agents (e.g., antigens, adjuvants), immune checkpoint inhibitors, and other therapeutic molecules, and have been shown to amplify and modulate immune responses. However, there are currently no NP-based tumor vaccines that are approved for clinical use due to the inability to precisely control design parameters, poor immunogenicity, challenges with manufacturing, and their inherent safety. Moreover, the precise and modular control over NP properties is a key requirement in order to test and understand the behavior of NP-based vaccines at the immune system/cancer interface (Bastings, M M C, et al. Nano Letters 2018 18:3557-3564; Hickey, J W, et al. Nano Lett. 2017 17:7045-7054; Mccarthy, D P, et al. Nanomedicine and Nanobiotechnology 2014 6:298-315; Chen, Z, et al. Nanoscale 2017 9:18129-18152). DNA origami (DO) is molecular self-assembly process that allows for a highly programable and precise DNA-based NPs (DNA-NPs), allows for the unprecedented control over NP geometry, site-specific incorporation of therapeutic molecules, and programable delivery (Bastings, M M C, et al. Nano Letters 2018 18:3557-3564; Halley, P D, et al. Small 2016 12:308-320; Afonin, K A, et al. Nature Nanotechnology 2013 8:296-304; Mikkila, J, et al. Nano Lett. 2014 14:2196-2200; DeLuca, M, et al. Nanoscale Horizons 2020; Campolongo, M J., et al. Advanced Drug Delivery Reviews 2010 62:606-616; Charbgoo, F, et al. Nanomedicine: Nanotechnology, Biology and Medicine 2018 14:685-697; Bath, J. & Turberfield, A J. Nature Nanotechnology 2007 2:275-284; Goltry, S, et al. Nanoscale 2015 7:10382-10390; Demanèche, S, et al. Appl. Environ. Microbiol. 2001 67:293-299; Dobrovolskaia, M A. DNA and RNA Nanotechnology 2016 3; Jeong, E H. et al. J Indust and Eng Chem 2017 56:55-61; Jiang, D. et al. ACS Appl. Mater. Interfaces 2016 8:4378-4384; Johnson, J A, et al. Nano letters 2019 19:8469-8475). DO is biocompatible, cheap to assemble, highly scalable, and allows for uniform assembly (Halley, P D, et al. Nano Research 2019 12:1207-1215). Previous studies have shown that DNA-NPs are internalized in a size and shape-dependent manner by APCs (Bastings, M M C, et al. Nano Letters 2018 18:3557-3564), enhance a pro-inflammatory immune response when conjugated with a CpG adjuvant in vitro (Schüller, V J, et al. ACS Nano 2011 5:9696-9702), can generate a long lasting immune response to a model antigen without an inherent response to the DNA-NPs in vivo (Schüller, V J, et al. ACS Nano 2011 5:9696-9702; Surana, S, et al. Nature Nanotechnology 2015 10:741-747). Collectively, these findings suggest that DO represents a promising novel platform for cancer vaccine development.

### Results

A rod-shaped Trojan ‘Horse’ DO nanostructure (92×12×15 nm) was produced using M13 scaffold DNA devoid of endotoxin (<9 EU/ml) and custom ‘staple’ oligonucleotides using standard DO construction methods (Halley, P. D. et al. Small 2016 12:308-320; Lucas, C R, et al. Small. 2022 e2108063). The DO was constructed as confirmed by gel electrophoresis and TEM. DO stability in human and murine plasma was tested up to 6 hrs. To address potential APC internalization of DO, murine splenic mononuclear cells were cultured with AlexaFluor-750 conjugated DO for various time points over a 24-hr time course. Flow cytometry analysis showed CD11b+, CD11c+, and CD19+cell populations showed time-dependent increases in the % of DO+ APCs suggesting effective internalization. To evaluate safety in vivo, ICR mice were injected with 5 doses (injections every 48 hr) of the following treatment groups: PBS (-ve control), CpG oligos (+vehicle control), scaffold DNA source material (p7249), and Horse DO at 12 mg/kg. No significant weight changes were observed. Biochemical panels on blood draws (day 10) revealed no toxicity of DO. Histopathology at end point (day 10) revealed only CpG treated mice exhibited mild hepatic necrosis in H&E stains of the liver. White blood cell counts suggested only CpG caused a marked immune response that dampened by day 10. Monocyte counts exhibited a minor increase in DO treated mice that returned below normal levels by day 10. Pro-inflammatory cytokine levels similarly showed a minor increase but returned to normal by day 10 suggesting DO are nontoxic and do not cause notable immunogenicity in vivo. Collectively, these data show the effective construction of a robust rod-shaped DO functionalized construction of a robust rod-shaped DO functionalized with peptides and oligonucleotides that is internalized by APCs ex vivo, non-toxic, and yields low immunogenicity in vivo. Thus, these findings provide strong rationale to incorporate tumor specific antigenic peptides and oligonucleotide adjuvants onto a single customizable DO vaccine delivery platform to mount an anti-cancer immune response mediated by cytotoxic T cells in vivo.

### Example 8

### Approach

This proposal employs a rod-shaped DO vehicle that includes 64 ‘overhang’ attachment sites previously described as the base DO nanovaccine (DO-VAC) platform, which allows for subsequent functionalization with up to 150 CpG adjuvant oligonucleotides and up to 100s of antigenic peptides via a novel electrostatic mediated attachment (FIG. 1). The DO-VAC will be internalized by APCs upon administration, processed, followed by antigenic presentation to CD8+ cytotoxic T cells, and subsequent antigen-specific T cell activation, expansion, and exhibition of cytotoxic anti-target/tumor cell effector activity (FIG. 1).

### DO-VAC Construction and Characterization

The rod-shaped DO-VAC vehicle was constructed containing 64 overhangs for CpG attachment using methods previously described, using p7249 M13 scaffold ssDNA purified via with the Endo-Free Maxi Prep method (Qiagen) containing low endotoxin levels (<2 EU/ml) followed by agarose gel electrophoresis (AGE) revealing well-formed DO nanostructures and effective CpG oligonucleotide attachment (FIG. 6A). DO-VAC vehicles were then functionalized with 10 lysine (K10) conjugated-OVA peptides and imaged via transmission electron microscopy (TEM) and atomic force microscopy (AFM), where micrographs showed well-folded and effectively antigen (Ag) functionalized DO (FIG. 6B). To evaluate higher numbers of attachment ‘arms/overhangs’, DO-VAC vehicles containing 30, 64, and 150 ‘arms’ were functionalized with CpG molecules where the number of attached molecules and efficiency were determined via nanodrop (FIG. 6E). Effective CpG adjuvant and K10-OVA peptide attachment to DO-VAC was further confirmed via fluorescent gel imaging (Atto-647-GpG, Cy3-DO, FAM-K10-OVA) (FIG. 7A). To evaluate DO-VAC stability in vitro, DO-VAC was mixed with 95% non-heat inactivated FBS where fluorescent gel imaging showed stable DO-VAC out to 24 hours (FIG. 8A).

### DO-VAC Cellular Uptake by Antigen Presenting Cells (APCs)

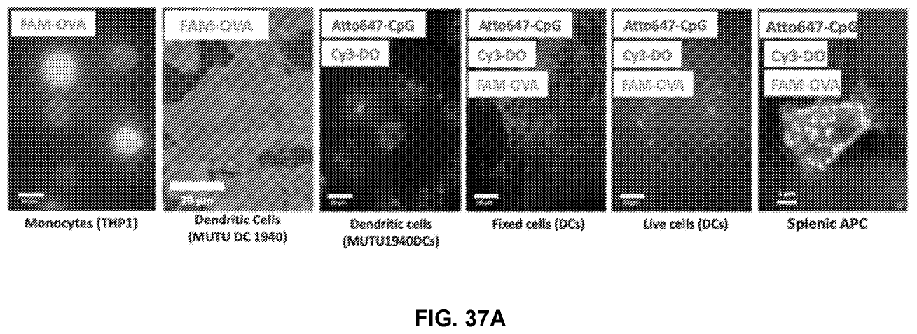

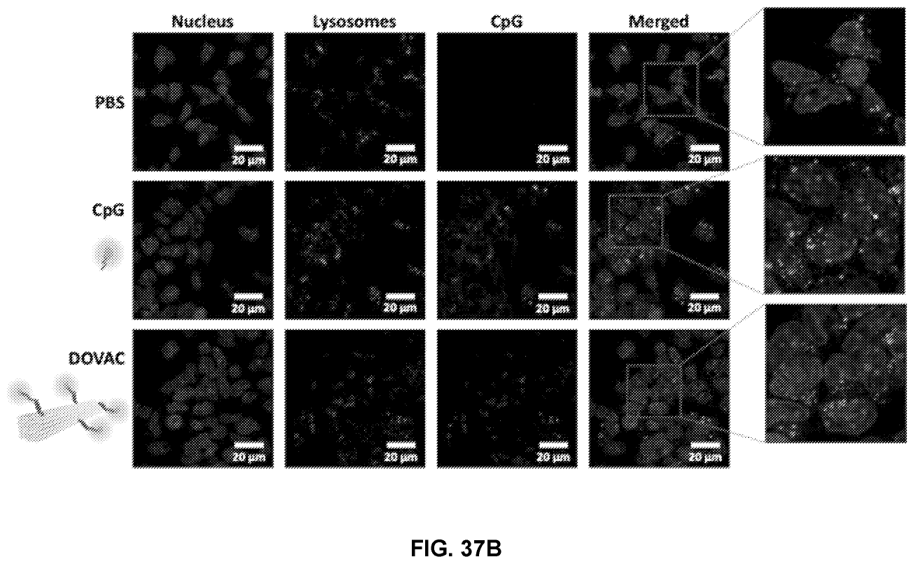

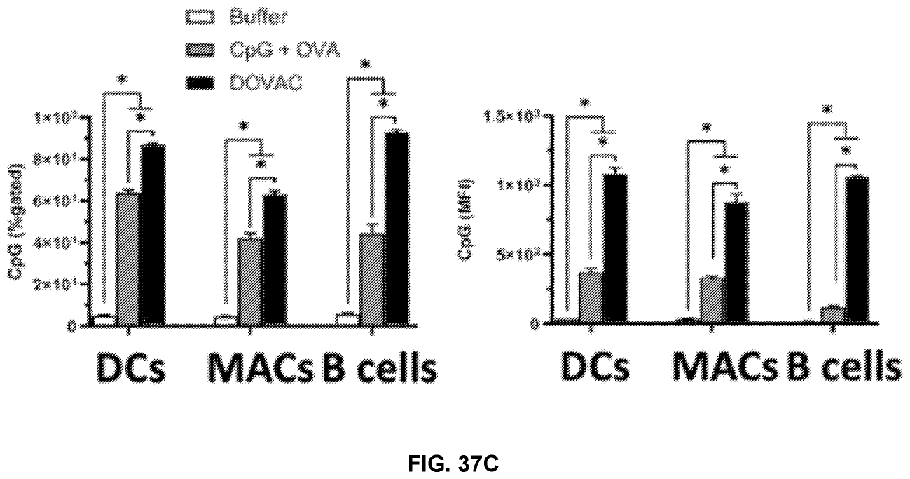

FAM-K10-OVA and DO-VAC cellular uptake was next evaluated across multiple cell types including THP1 (monocytes/AML cell line), Mutu 1940 dendritic cells (DCs), and murine primary splenic mononuclear APCs where cells were cultured overnight with FAM-K10-OVA or various versions of DO-VAC (Atto-647-GpG, Cy3-DO, FAM-K10-OVA) followed by fluorescent microscopy (FIG. 37A). FAM-K10-OVA was effectively taken up by THP1 and Mutu 1940 DCs, while DO-VAC was also up taken by Mutu 1940 DCs and splenic APCs in an effective manner as evidenced by multi-channel co-localization (FIG. 37A). These findings were extended using confocal microscopy showing effective cellular internalization by Mutu 1940 DCs (FIG. 37B) and by flow cytometry in primary splenic APCs (CD11c⁺ DCs, CD11b⁺ Macrophages, and CD19⁺ B cells), which revelated significant elevation of Atto-647-labeled DO-VAC relative to physical mixture of Atto-647 CpG and OVA (FIG. 37C) suggesting that DO-VAC enhances APC cellular uptake of vaccine molecular payloads.

### DO-VAC Influenced Antigen Presentation and Activation in APCs

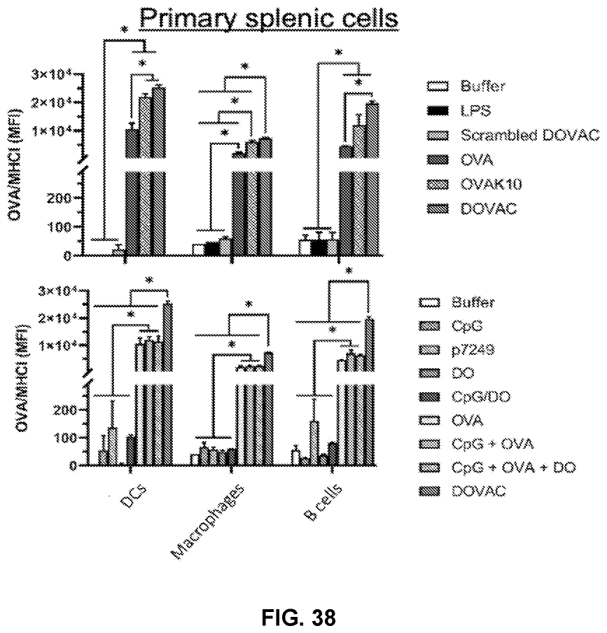

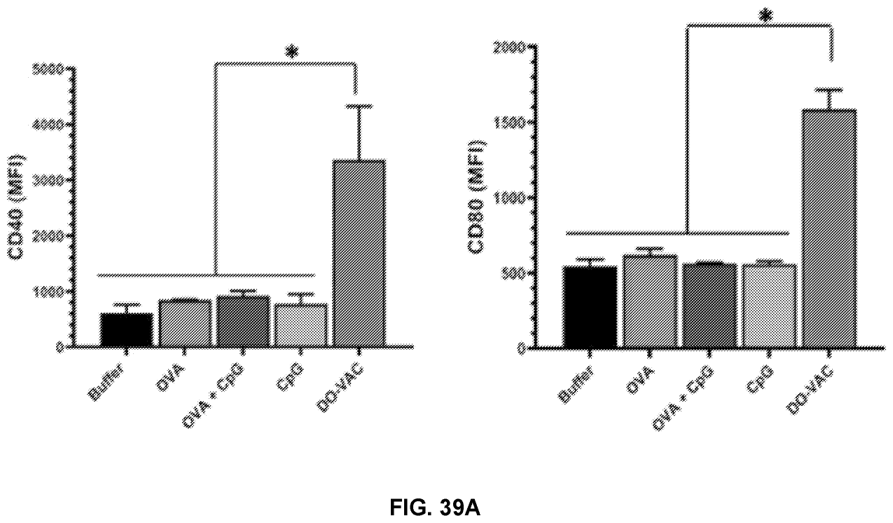

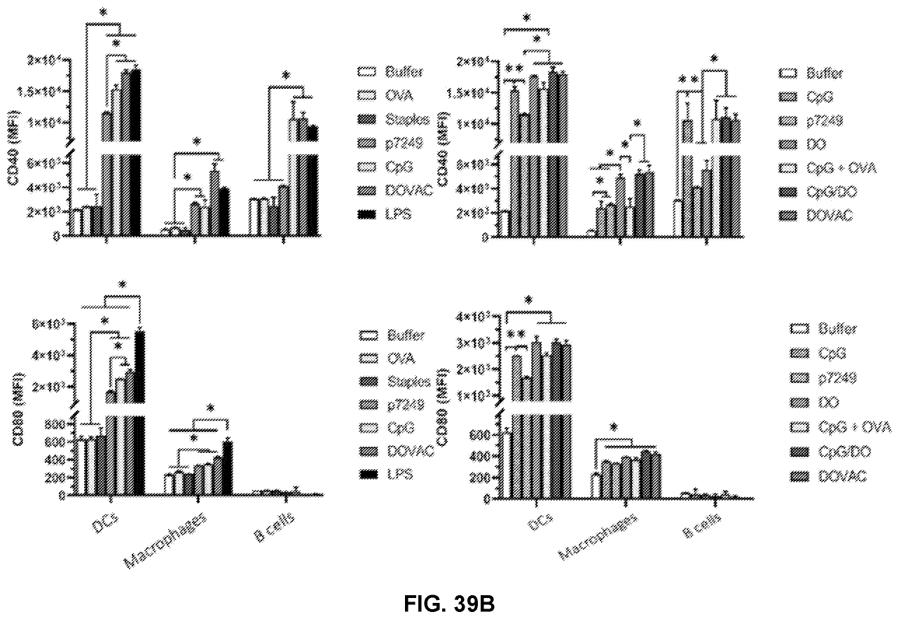

Next evaluated was antigen presentation by APCs cultured with DO-VAC relative to antigen/adjuvant physical mixtures in Mutu 1940 DCs, where OVA/MHCl surface presentation was significantly elevated among Mutu 1940 DCs that were treated with DO-VAC relative to physical mixture formulations as shown by flow cytometry (FIG. 9A). These findings were extended into murine splenic APCs that were cultured with a variety of control conditions including LPS, scrambled K10-OVA peptide sequences, OVA or K10-OVA alone, CpG, p7249 scaffold only, and multiple physical OVA peptide antigen and adjuvant conditions. DO-VAC treated APCs showed the highest level of OVA/MHCl surface presentation across all APC cell types, and significant elevations over numerous physical mixtures, with the exception of K10-OVA in Macrophages and B cells, and over all conditions that lacked OVA antigenic peptide (FIG. 38). DO-VAC mediated APC activation relative to the same conditions described in (FIG. 38) was also evaluated by monitoring CD40 and CD80 co-stimulatory molecule expression levels by flow cytometry in Mutu 1940 DCs and murine splenic primary APCs. The data showed significant elevations of CD40 and CD80 levels on the Mutu 1940 DC cell surface on cells treated with DO-VAC relative to physical mixture vaccine formulations (FIG. 39A). In primary APCs, all conditions except for OVA only and staple oligonucleotide elevated CD40 surface levels, as well as DC CD80 levels, and to a lesser extent on Macrophages and little elevation on B cells (FIG. 39B). Importantly, all expected adjuvants (nucleic acid and LPS) induced a DC activation phenotype as expected, as DCs are the primary APCs related to vaccine efficacy. Taken together, these findings suggest that the DO-VAC vehicle allows for optimal antigen presentation and activation on the APC surface relative to free antigen/adjuvant vaccine formulations.

### DO-VAC Performance Evaluation In Vivo

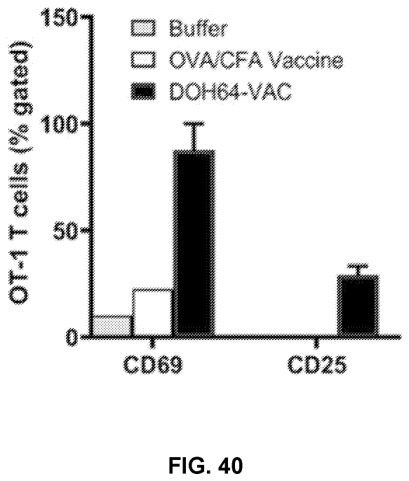

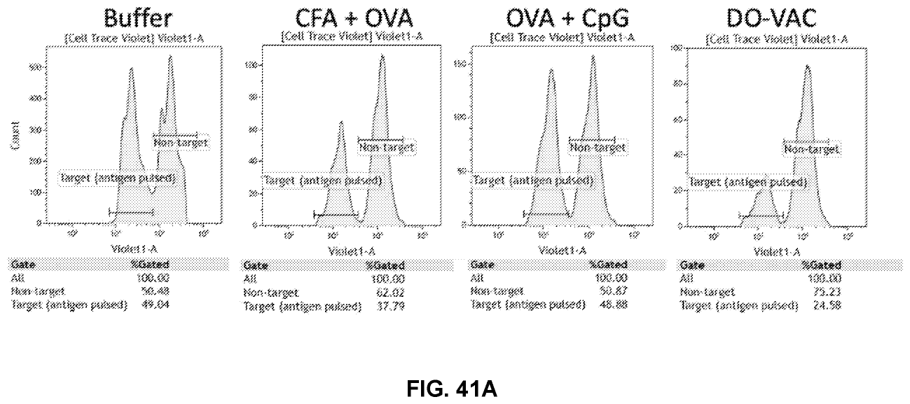

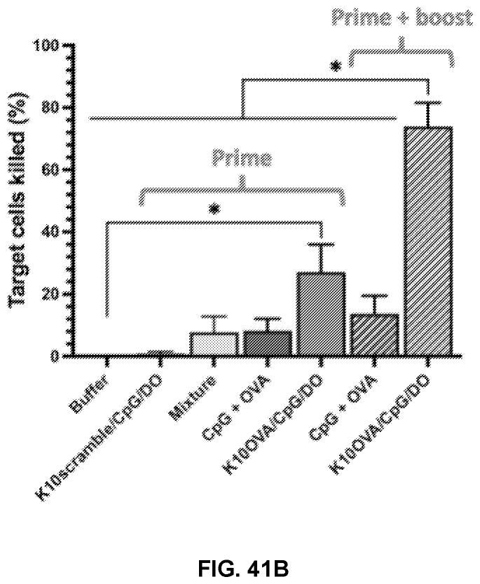

To begin to evaluate DO-VAC performance in vivo, T cell activation and proliferation was assessed by adoptively transferring OT-1 (OVA peptide (SIINFEKL)) transgenic TCR T cells into C57/Bl6 recipient mice. The mice were immunized (s.c.) with OVA peptides/CFA physical mixture or DO-VAC (DO/CpG/OVA peptides) followed by T cell activation monitoring after 24 hours by flow cytometry. DO-VAC treated mice show elevated levels of CD69 and CD25 (FIG. 40) on the OT-1 T cell surface showing effective activation. Mice were also injected with Violet Proliferation dye labeled OT-1 T cells and immunized with either 2× OVA+CpG or 1× OVA DO-VAC, where antigen specific proliferation was enhanced among DO-VAC treated mice. To study DO-VAC efficacy in vivo, mice were immunized (i.m.) with CFA/OVA-K10, OVA-K10+CpG, DO+OVA-K10+CpG, or DO-VAC (s.c.). On day 7, Cell trace violet-labeled (low) C57/Bl6 splenic mononuclear cells were pulsed with OVA peptide antigen, while Cell trace violet-labeled (high) were not, were mixed, and were adoptively transferred into C57/Bl6 recipient mice. Splenic mononuclear cells were collected 20 hours later and evaluated for viability by flow cytometry. DO-VAC treated mice exhibited the highest degree of target cell killing (FIGS. 41A-41B) where DO-VAC significantly outperformed not only the CpG+OVA peptide physical mixture, but also the standard CFA adjuvant+OVA peptide. To extend these findings, animals were immunized either on day 0 or days 0 and 7 (prime+boost regimen), followed by antigen-pulsed cell challenge as described above where DO-VAC significantly outperformed CpG+OVA peptide physical mixture vaccine formulation on both the prime only and prime+boost regimen (FIG. 41C). Together, these findings suggest that DO-VAC effectively activates and expands CD8+ T cells in an antigen specific manner and displays superior efficacy relative to adjuvant/antigen physical mixture vaccine and the CFA standard adjuvant.

### Pre-Evaluation of a DC-Targeted DO-VAC

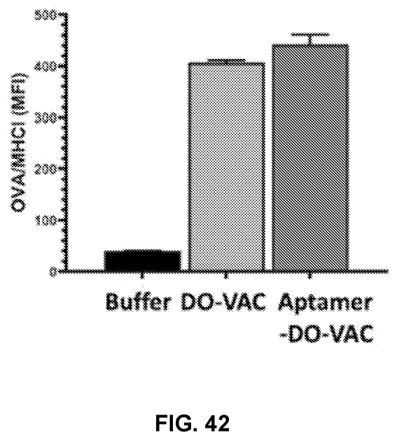

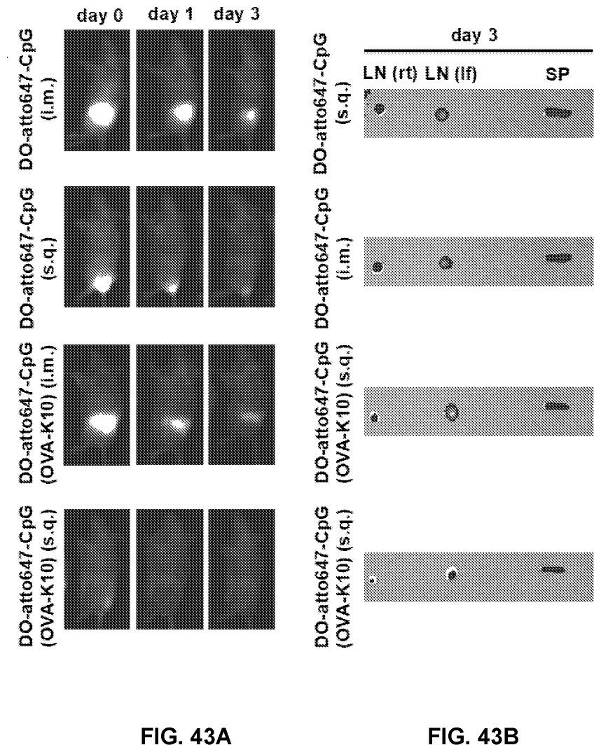

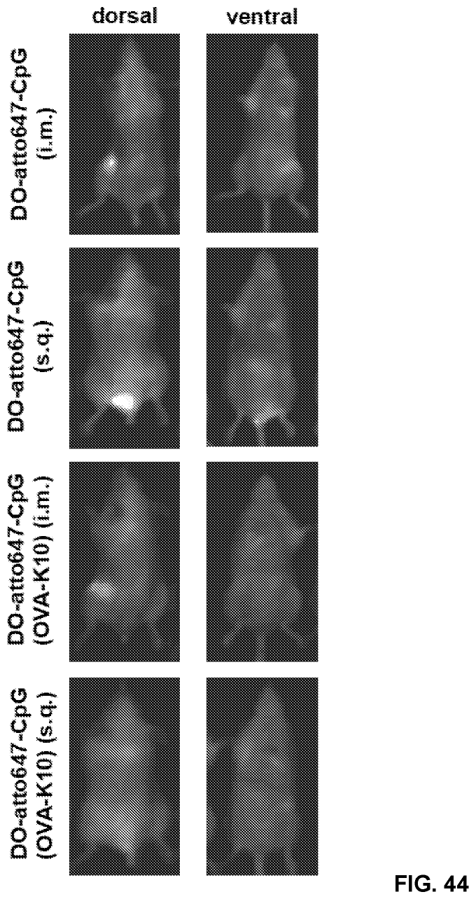

Although our DO-VAC displays superior efficacy over physical mixture formulations of antigen/adjuvant, whether we can improve therapeutic efficacy by targeted delivery to APCs remains unclear. To address this, the DO-VAC platform was functionalized with various levels of Aptamer oligonucleotide sequences specific for the DEC205 receptor on the DC cell surface followed by AGE where a gel shift indicated effective attachment (FIG. 42A). DEC205 aptamer-DO-VAC mediated enhanced delivery to Mutu 1940 DCs was confirmed by flow cytometry (FIG. 42B) showing a modest DEC205-aptamer induced increase in the level of OVA peptide/MHCl expression (FIG. 42C), suggesting thus far that a DC targeted DO-VAC nanovaccine platform may be functional.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A vaccine device, comprising a DNA origami nanostructure formed from a plurality of scaffold strands and a plurality of staple strands assembled into a rod shape, wherein a peptide antigen is attached to the DNA of the nanostructure by electrostatic interaction.

2. The vaccine device of claim 1, wherein the peptide antigen comprises at least 5, 6, 7, 8, 9, or 10 contiguous positively charged amino acids.

3. The vaccine device of claim 2, wherein the at least 5, 6, 7, 8, 9, or 10 continuous positively charged amino acids are at the N-terminus of the peptide antigen.

4. The vaccine device of claim 2, wherein the positively charged amino acids are lysine or arginine amino acids.

5. The vaccine device of claim 2, wherein the peptide antigen comprises at least 10 contiguous lysine amino acids.

6. The vaccine device of claim 2, wherein the peptide antigen comprises at least 10 contiguous arginine amino acids.

7. The vaccine device of claim 1, wherein at least 1,000 peptide antigens are attached to the DNA nanostructure.

8. The vaccine device of claim 1, comprising at least 30, 35, 40, 45, 50, 60, 70, 80, 90, 100 peptides per nm².

9. The vaccine device of claim 1, comprising at least 50, 60, 70, 80, 90, 100 nM peptide.

10. The vaccine device of claim 1, wherein each of the plurality of scaffold strands are 5,000 to 10,000 nucleotides in length,

11. The vaccine device of claim 1, wherein each of the plurality of scaffold strands are derived from a virus.

12. The vaccine device of claim 1, wherein each of the plurality of scaffold strands are derived from bacteriophage M13.

13. The vaccine device of claim 1, wherein the peptide antigen comprises a viral antigen.

14. The vaccine device of claim 1, wherein the peptide antigen comprises a tumor specific antigen and/or tumor associated antigen.

15. The vaccine device of claim 1, wherein the DNA origami nanostructure comprises one or more first single stranded DNA oligonucleotide attachment arms configured to bind to a first complementary DNA oligonucleotide strands.

16. The vaccine device of claim 1, wherein the DNA origami nanostructure is encapsulated in an alginate capsule.

17. A method for vaccinating a subject, comprising administering to the subject the vaccine device of claim 1.
